(19) United States
(12) Patent Application Publication
(10) Pub. No.: US 2024/0239875 A1
(43) Pub. Date: Jul. 18, 2024

(54) SARS-COV-2 SPIKE PROTEIN ANTIBODIES

(71) Applicant: CURIA IP HOLDINGS, LLC, Albany, NY (US)

(72) Inventors: Brian A. Zabel, Albany, NY (US); Xiaomei Ge, Albany, NY (US); Dan Luo, Albany, NY (US); Ling Zhang, Albany, NY (US); Vydehi Kanneganti, Albany, NY (US); Joyce Yu, Albany, NY (US); Hsunhui Yang, Albany, NY (US); Lequn Zhao, Albany, NY (US); Hua Tu, Albany, NY (US)

(21) Appl. No.: 18/561,635

(22) PCT Filed: May 17, 2022
(86) PCT No.: PCT/US2022/029680

§ 371 Date: Nov. 16, 2023

Related U.S. Application Data
(60) Provisional application No. 63/189,635, filed on May 17, 2021.
(60) Provisional application No. 63/216,406, filed on Jun. 29, 2021.

(51) Int. Cl. C07K 16/10; A61P 31/14; G01N 33/569

(52) U.S. Cl. CPC C07K 16/1003; A61P 31/14; G01N 33/56983; C07K 2317/24; C07K 2317/622; C07K 2317/76; G01N 2333/165; G01N 2469/10

(57) ABSTRACT

The present disclosure provides antibodies that bind to the SARS-CoV-2 spike protein, as well as compositions containing the same, and methods of making and using such a composition for treating, preventing, and/or detecting SARS-CoV-2 infection.

This application claims the priority benefit of U.S. Provisional Patent Application Ser. Nos. 63/189,635, filed May 17, 2021, and 63/216,406, filed Jun. 29, 2021, each of which is hereby incorporated by reference in its entirety.

### BACKGROUND OF THE INVENTION

Coronaviruses (CoV) historically are known to cause relatively mild upper respiratory tract infections, and account for approximately 30% of the cases of the common cold in humans. In late December 2019, a novel coronavirus, currently named as severe acute respiratory syndrome coronavirus 2 (SARS-CoV-2), emerged from Wuhan, China, and resulted in significant outbreaks in 216 countries, with over 159M reported cases and over 3.3M deaths (WHO, May 10, 2021). The disease was officially named Coronavirus Disease-2019 (COVID-19, by WHO on Feb. 11, 2020). COVID-19 is a potential zoonotic disease with a mortality rate estimated from 2%-5%. Currently, there is no definite treatment for COVID-19 although treatments are being investigated in over 1100 clinical trials (https://clinicaltrials.gov/ct2/who_table).

A related CoV was described in 2003, causing severe acute respiratory syndrome coronavirus (SARS-CoV) characterized by severe respiratory distress in humans leading to mortality in 9.6% of individuals infected (Baker, S. C. 2004. Coronaviruses from common colds to severe acute respiratory syndrome. Pediatr. Infect. Dis. J. 23: 1049-1050). In the year 2003, SARS-CoV established efficient human to human transmission resulting in several super-spreading events. By the end of the outbreak in July of 2003, SARS-CoV was responsible for more than 774 deaths and 8096 cases worldwide involving 29 countries (see World Health Organization website, Epidemic and Pandemic Alert and Response, Diseases, SARs). Since the conclusion of the SARS outbreak several reports of confirmed cases of SARS of unknown origin (see World Health Organization website) indicate that the environmental threat of SARS-CoV still exists. SARS-CoV-like virus can be isolated from horseshoe bats in China, and researchers postulate that this is the natural reservoir for the virus (Li, W., et al. 2005. Bats are the natural reservoirs of SARS-like coronaviruses. Science. 310: 676-679). SARS-CoV-like virus remains present in intermediate wild animal hosts, such as the Himalayan palm civet, raising the possibility of re-emergence of SARS-CoV infection in humans. Because of the remaining threat, it is prudent to develop effective modalities of pre- and post-exposure treatments against SARS-CoV infection.

Numerous clinical studies on treatment of SARS-CoV-2 are ongoing (https://clinicaltrials.gov/ct2/results?cond=%22wuhan+coronavirus %22). SARS-CoV-2 sequences are known (https://www.ncbi.nlm.nih.gov/genbank/sars-cov-2-seqs/). Currently, 18 studies investigating the efficacy of convalescent plasma as a therapeutic modality are underway (https://clinicaltrials.gov/ct2/who_table).

Despite the focus and energy directed at this novel disease, a cure has yet to be discovered and new therapeutic modalities are urgently needed. As is widely recognized, the design, testing and marketing approval of new antiviral agents takes years. Thus, therapeutic and prophylactic modalities leveraging off experience with safe and efficacious agents are in great current need and of significant interest.

### BRIEF SUMMARY OF THE INVENTION

In various embodiments, the present invention meets this need by providing a new anti-CoV-S antibody that can prevent, treat and/or detect SARS-CoV-2 infection, and methods of making and administering this agent to subjects in need thereof.

In some embodiments, the present invention provides antigen binding domains, including antibodies, which bind to CoV-S, comprising the vhCDR1, vhCDR2, vhCDR3, vlCDR1, vlCDR2 and vlCDR3 sequences from an antibody selected from the group consisting of clone IDs: 1-B11-A, 1-L10-A, 2-H7-A, 2-J9-A, 2-O12-A, 2-P2-A, 3-E13-A, 3-P7-A, 4-A15-A, 4-C3-A, 4-K13-A, 4-L4-A, 5-H22-A, 5-P24-A, 6-O12-A, 8-N24-A, 9-J11-A, 9-K4-A, 9-L13-A, 9-P9-A, 10-B11-A, 10-B13-A, 10-L12-A, 10-L24-A, 10-O24-A, 10-O3-A, 4-M3-A, 4-N22-A, 7-B10-A, 8-H5-A, 2-G20-A, 3-E2-A, 4-K16-A, 6-C19-A, 6-L8-A, 7-D7-A, 7-N20-A, 8-A17-A, 8-H3-A, 8-L17-A, 9-F6-A, and 10-I12-A (as depicted in FIG. 12A-12PP).

In some embodiments, the present invention provides anti-CoV-S antigen binding domains (including antibodies) comprising the variable heavy domain (VH) and variable light domain (VL) from an antibody selected from the group consisting of clone IDs: 1-B11-A, 1-L10-A, 2-H7-A, 2-J9-A, 2-O12-A, 2-P2-A, 3-E13-A, 3-P7-A, 4-A15-A, 4-C3-A, 4-K13-A, 4-L4-A, 5-H22-A, 5-P24-A, 6-O12-A, 8-N24-A, 9- J11-A, 9-K4-A, 9-L13-A, 9-P9-A, 10-B11-A, 10-B13-A, 10-L12-A, 10-L24-A, 10-O24-A, 10-O3-A, 4-M3-A, 4-N22-A, 7-B10-A, 8-H5-A, 2-G20-A, 3-E2-A, 4-K16-A, 6-C19-A, 6-L8-A, 7-D7-A, 7-N20-A, 8-A17-A, 8-H3-A, 8-L17-A, 9-F6-A, and 10-I12-A (as depicted in FIG. 12A-12PP).

In some embodiments, the present invention provides anti-CoV-S antigen binding domains (including antibodies) selected from the group consisting of clone IDs: 1-B11-A, 1-L10-A, 2-H7-A, 2-J9-A, 2-O12-A, 2-P2-A, 3-E13-A, 3-P7-A, 4-A15-A, 4-C3-A, 4-K13-A, 4-L4-A, 5-H22-A, 5-P24-A, 6-O12-A, 8-N24-A, 9-J11-A, 9-K4-A, 9-L13-A, 9-P9-A, 10-B11-A, 10-B13-A, 10-L12-A, 10-L24-A, 10-O24-A, 10-O3-A, 4-M3-A, 4-N22-A, 7-B10-A, 8-H5-A, 2-G20-A, 3-E2-A, 4-K16-A, 6-C19-A, 6-L8-A, 7-D7-A, 7-N20-A, 8-A17- A, 8-H3-A, 8-L17-A, 9-F6-A, and 10-I12-A (as depicted in FIG. 12A-12PP).

In some embodiments, the present invention provides an antigen binding domain (including antibodies) that competes with the antibodies or antigen-binding domains discussed above or herein for binding to CoV-S.

In some embodiments, the present invention provides a pharmaceutical composition and formulation comprising an isolated antibody, as discussed above or herein, and a pharmaceutically acceptable carrier or diluent.

In some embodiments, the present invention provides nucleic acid compositions comprising: a) a first nucleic acid encoding the heavy chain variable domain comprising the vhCDR1, vhCDR2 and vhCDR3 from an antibody; and b) a second nucleic acid encoding a light chain variable domain comprising vlCDR1, vlCDR2 and vlCDR3 from an antibody selected from the group consisting of clone IDs: 1-B11-A, 1-L10-A, 2-H7-A, 2-J9-A, 2-O12-A, 2-P2-A, 3-E13-A, 3-P7-A, 4-A15-A, 4-C3-A, 4-K13-A, 4-L4-A, 5-H22-A, 5-P24-A, 6-O12-A, 8-N24-A, 9-J11-A, 9-K4-A, 9-L13-A, 9-P9-A, 10-B11-A, 10-B13-A, 10-L12-A, 10-L24-A, 10-O24-A, 10-O3-A, 4-M3-A, 4-N22-A, 7-B10-A, 8-H5-A, 2-G20-A, 3-E2-A, 4-K16-A, 6-C19-A, 6-L8-A, 7-D7-A, 7-N20-A, 8-A17-A, 8-H3-A, 8-L17-A, 9-F6-A, and 10-I12-A (as depicted in FIG. 12A-12PP).

In some embodiments, the present invention provides nucleic acid compositions comprising: a) a first nucleic acid encoding the heavy chain variable domain (VH); and b) a second nucleic acid encoding a light chain variable domain (VL), wherein the heavy and light chain variable domains are from an antibody selected from the group consisting of clone IDs: 1-B11-A, 1-L10-A, 2-H7-A, 2-J9-A, 2-O12-A, 2-P2-A, 3-E13-A, 3-P7-A, 4-A15-A, 4-C3-A, 4-K13-A, 4-L4-A, 5-H22-A, 5-P24-A, 6-O12-A, 8-N24-A, 9-J11-A, 9-K4-A, 9-L13-A, 9-P9-A, 10-B11-A, 10-B13-A, 10-L12-A, 10-L24-A, 10-O24-A, 10-O3-A, 4-M3-A, 4-N22-A, 7-B10-A, 8-H5-A, 2-G20-A, 3-E2-A, 4-K16-A, 6-C19-A, 6-L8-A, 7-D7-A, 7-N20-A, 8-A17-A, 8-H3-A, 8-L17-A, 9-F6-A, and 10-I12-A (as depicted in FIG. 12A-12PP).

In some embodiments, the present invention provides expression vectors comprising the first and/or second nucleic acids as outlined herein and above.

In some embodiments, the present invention provides host cells comprising the expression vector compositions, either as single expression vectors or two expression vectors.

In some embodiments, the present invention provides methods of making an anti-CoV-S antibody comprising a) culturing a host cell of the invention with expression vector(s) under conditions wherein the antibody is produced; and b) recovering the antibody.

In some embodiments, the present invention provides methods for treating SARS-CoV-2 infection comprising administering an antibody as discussed above or herein to a patient in need.

In some embodiments, the present invention provides methods for preventing SARS-CoV-2 infection comprising administering an antibody as discussed above or herein to a patient in need.

In some embodiments, the present invention provides methods for detecting SARS-CoV-2 in a human sample.

In some embodiments, the method for detecting comprises contacting the human sample with the antibody of any one of the preceding claims, and detecting binding of the antibody to SARS-CoV-2 spike protein (CoV-S) as an indication of presence of SARS-CoV-2 in the sample.

### BRIEF DESCRIPTION OF THE DRAWINGS

FIG. 1 illustrates the primary amino acid sequence (SEQ ID NO:1) of a SARS-CoV-2 prefusion stabilized trimer protein immunogen that was derived from the SARS-CoV-2 of WIV02 isolate (see Genbank Reference No. MN996527.1, which is hereby incorporated by reference in its entirety). The fusion polypeptide includes an N-terminal signal sequence, SARS-CoV-2 spike protein bearing five substitutions (R691G, R692S, R694A, K995P, V996P), a T4 fibritin trimerization domain, followed by an HRV3C cleavage site, and a C-terminal His8 tag.

FIG. 2A-2C provide a comprehensive analytic summary of 42 SARS-CoV-2 spike binding mAbs. In FIG. 2C, the HC-CDR3 and LC-CDR3 amino acid sequences of RBD-binding antibodies are shown as follows: 10-B13-A (SEQ ID NOS: 218 and 221, respectively); 9-L13-A (SEQ ID NOS: 188 and 191, respectively); 10-O24-A (SEQ ID NOS: 248 and 251, respectively); 10-L12-A (SEQ ID NOS: 228 and 231, respectively); 9-K4-A (SEQ ID NOS: 178 and 181, respectively); 3-P7-A (SEQ ID NOS: 78 and 81, respectively); 5-P24-A (SEQ ID NOS: 138 and 141, respectively); 10-L24-A (SEQ ID NOS: 238 and 241, respectively); 2-O12-A (SEQ ID NOS: 48 and 51, respectively); 3-E2-A (SEQ ID NOS: 318 and 321, respectively); 4-K13-A (SEQ ID NOS: 108 and 111, respectively); 4-L4-A (SEQ ID NOS: 118 and 121, respectively); 5-H22-A (SEQ ID NOS: 128 and 131, respectively); 2-H7-A (SEQ ID NOS: 28 and 31, respectively); 8-H3-A (SEQ ID NOS: 388 and 391, respectively); 8-L17-A (SEQ ID NOS: 398 and 401, respectively); 7-B10-A (SEQ ID NOS: 288 and 291, respectively); 4-N22-A (SEQ ID NOS: 278 and 281, respectively); 4-M3-A (SEQ ID NOS: 268 and 271, respectively); 8-H5-A (SEQ ID NOS: 298 and 301, respectively); and 8-N24 (SEQ ID NOS: 158 and 161, respectively). In FIG. 2C, the HC-CDR3 and LC-CDR3 amino acid sequences of S1 non-RBD-binding antibodies are shown as follows: 10-O3-A (SEQ ID NOS: 258 and 261, respectively); 4-A15-A (SEQ ID NOS: 88 and 91, respectively); 4-K16-A (SEQ ID NOS: 328 and 331, respectively); 4-C3-A (SEQ ID NOS: 98 and 101, respectively); and 6-L8-A (SEQ ID NOS: 348 and 351, respectively). In FIG. 2C, the HC-CDR3 and LC-CDR3 amino acid sequences of S2-binding antibodies are shown as follows: 10-B11-A (SEQ ID NOS: 208 and 211, respectively); 2-P2-A (SEQ ID NOS: 58 and 61, respectively); 3-E13-A (SEQ ID NOS: 68 and 71, respectively); 6-C19-A (SEQ ID NOS: 338 and 341, respectively); 2-J9-A (SEQ ID NOS: 38 and 41, respectively); 9-P9-A (SEQ ID NOS: 198 and 201, respectively); 1-B11-A (SEQ ID NOS: 8 and 11, respectively); and 10-I12-A (SEQ ID NOS: 418 and 421, respectively). In FIG. 2C, the HC-CDR3 and LC-CDR3 amino acid sequences of non-RBD, non-S1, and non-S2 binding antibodies are shown as follows: 6-O12-A (SEQ ID NOS: 148 and 151, respectively); 1-L10-A (SEQ ID NOS: 18 and 21, respectively); 2-G20-A (SEQ ID NOS: 308 and 311, respectively); 7-D7-A (SEQ ID NOS: 358 and 361, respectively); 8-A17-A (SEQ ID NOS: 378 and 381, respectively); and 9-F6-A (SEQ ID NOS: 408 and 411, respectively). In FIG. 2C, the HC-CDR3 and LC-CDR3 amino acid sequences of SARS-CoV-2 spike-selective antibodies are shown as follows: 7-N20-A (SEQ ID NOS: 368 and 371, respectively) and 9-J11-A (SEQ ID NOS: 168 and 171, respectively).

FIGS. 3A-3D is a panel of graphs depicting EC₅₀ ELISA binding curves for selected SARS-CoV-2 spike-binding mAbs against spike trimer, S2 domain, RBD domain, and S1 domain, respectively.

FIGS. 4A-4D is a panel of graphs depicting EC₅₀ ELISA binding curves for selected SARS-CoV-2 spike-binding mAbs against spike trimers from SARS-CoV-1, HKU1, HCOV-OC43, and MERS, respectively.

FIG. 5 is a graph depicting IC₅₀ ELISA neutralization curves for selected SARS-CoV-2 spike-binding mAbs inhibiting the binding of SARS-CoV-2 spike trimer to huACE2.

FIG. 6 is a panel of graphs depicting IC₅₀ titration of 5-P24-A, 3-E2-A, and 8-H3-A in SARS-CoV-2 pseudovirus

FIG. 7 is a graph depicting IC₅₀ titration of 10-B13-A (human Fc IgG2 chimera) in SARS-CoV-1 pseudovirus ACE2+TMPRSS2+ target cell infection assay.

FIG. 8 includes a graph depicting IC₅₀ titration of 10-B13-A (human Fc IgG2 chimera) in BSL3 Vero E6 infection plaque assay, with corresponding images of plaque assay results depicted.

FIG. 9 depicts binding kinetics for selected SARS-CoV-2 spike-binding mAbs against RBD.

FIG. 10 is an illustrative binding and functional summary of 42 SARS-CoV-2 spike binding mAbs.

FIG. 11 illustrates a SARS-CoV-2 spike binding mAb dendrogram.

FIG. 12A-PP illustrate amino acid and nucleotide sequences of exemplary SARS-CoV-2 spike binding mAbs provided herein. Although the IMGT numbering scheme was used to designate the complementarity determining regions of the variable domains, it is also contemplated that alternative numbering schemes—including Kabat, Chothia, Martin, Gelfand, or Honneger—can be used to identify complementarity determining regions. See Dondelinger et al., “Understanding the Significance and Implications of Antibody Numbering and Antigen-Binding Surface/Residue Definition,” Frontiers in Immunol. 9:2278 (2018), which is hereby incorporated by reference in its entirety.

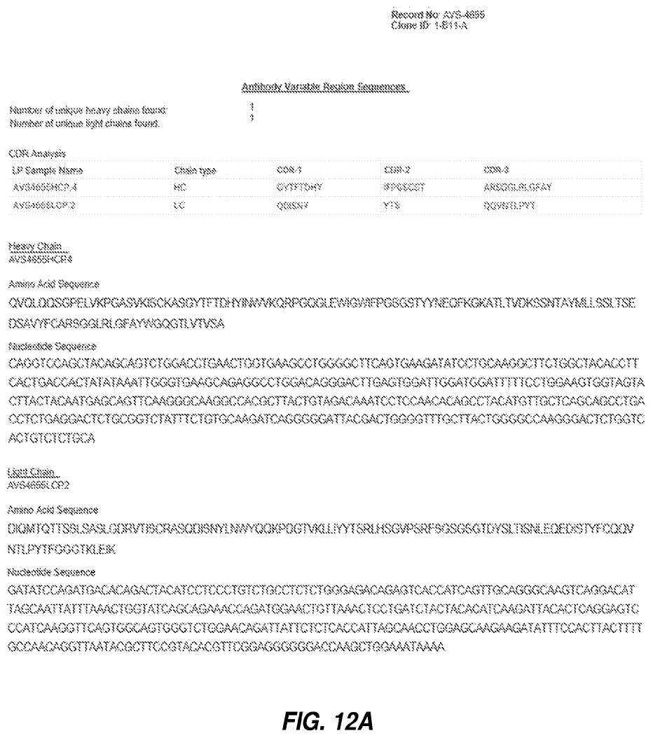

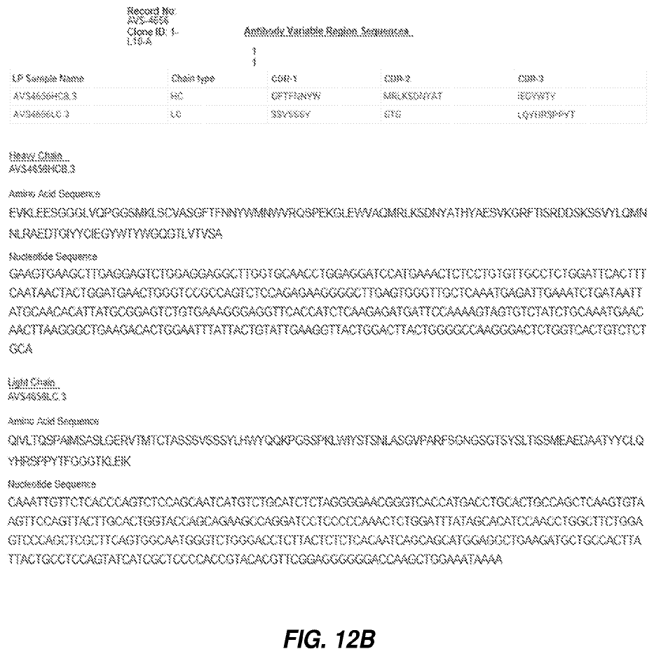

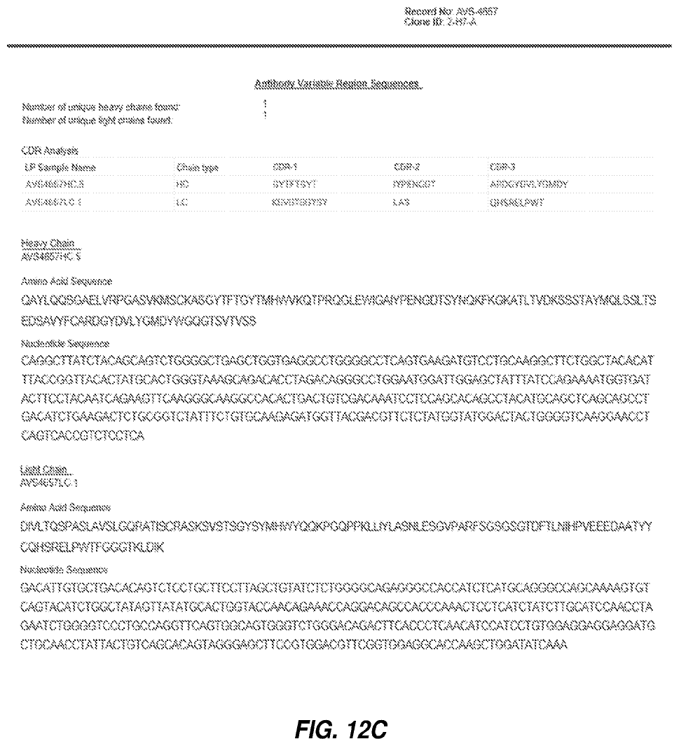

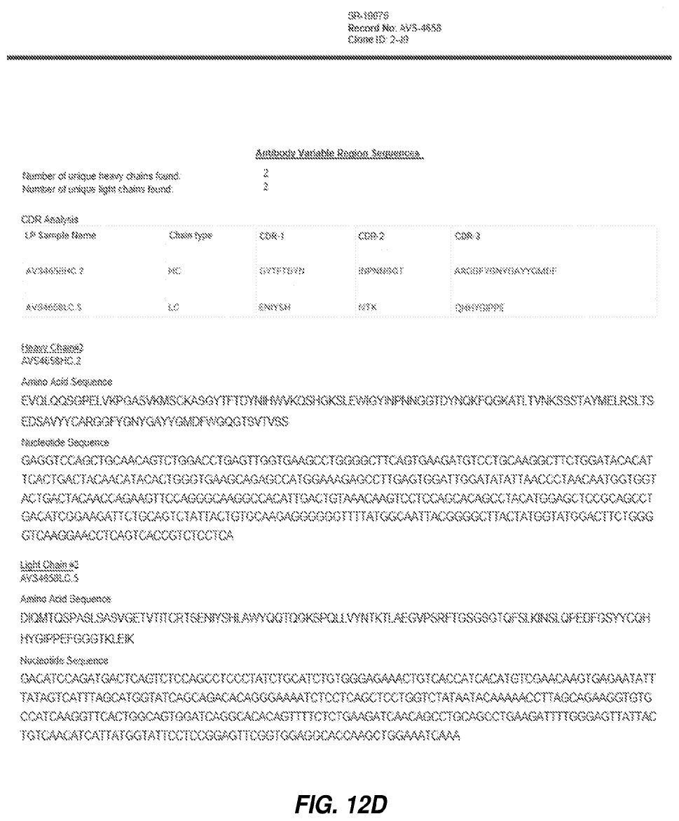

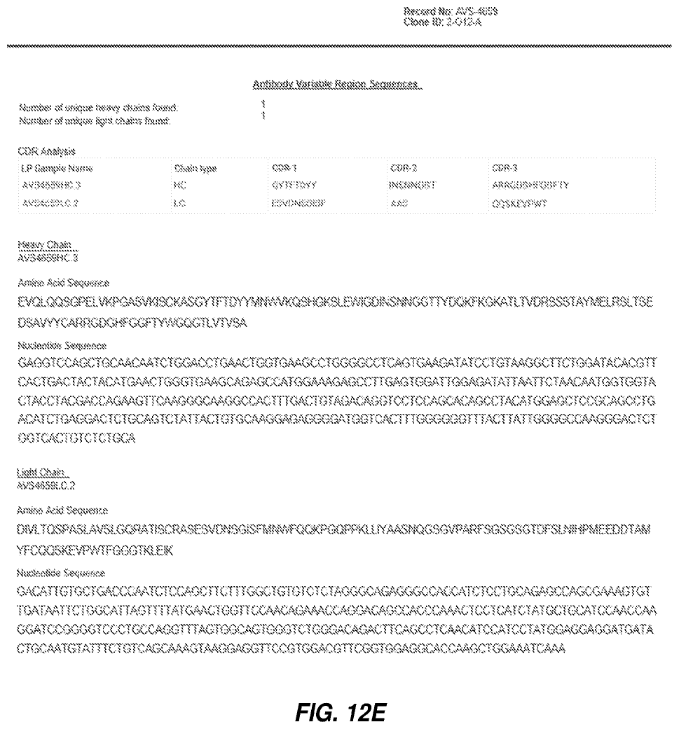

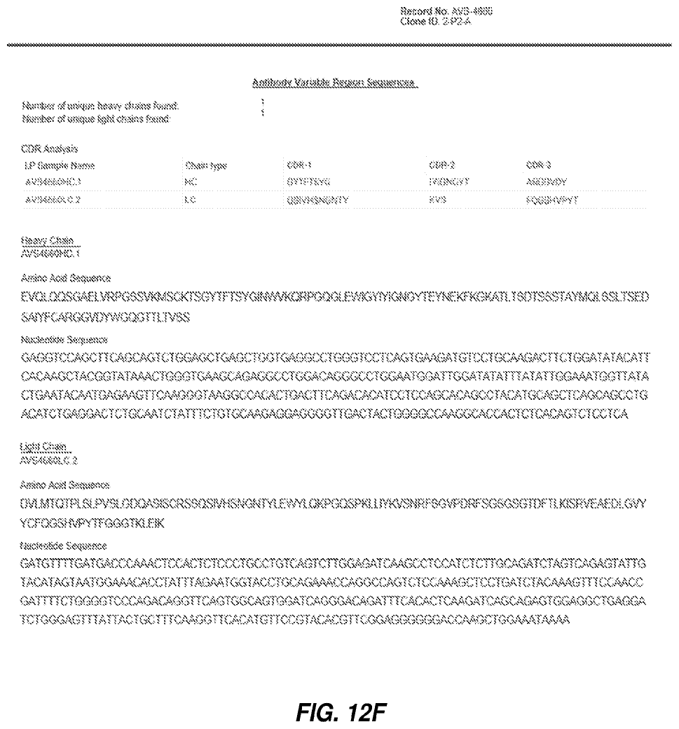

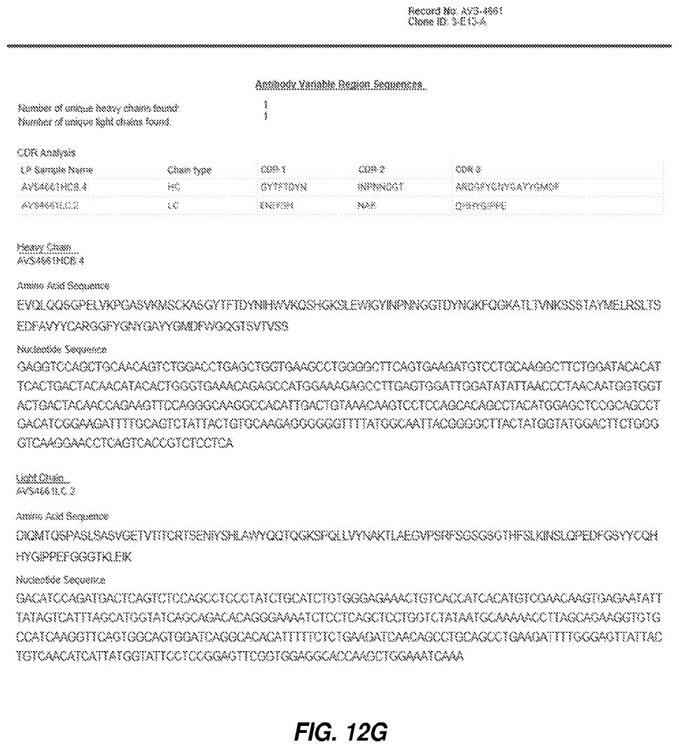

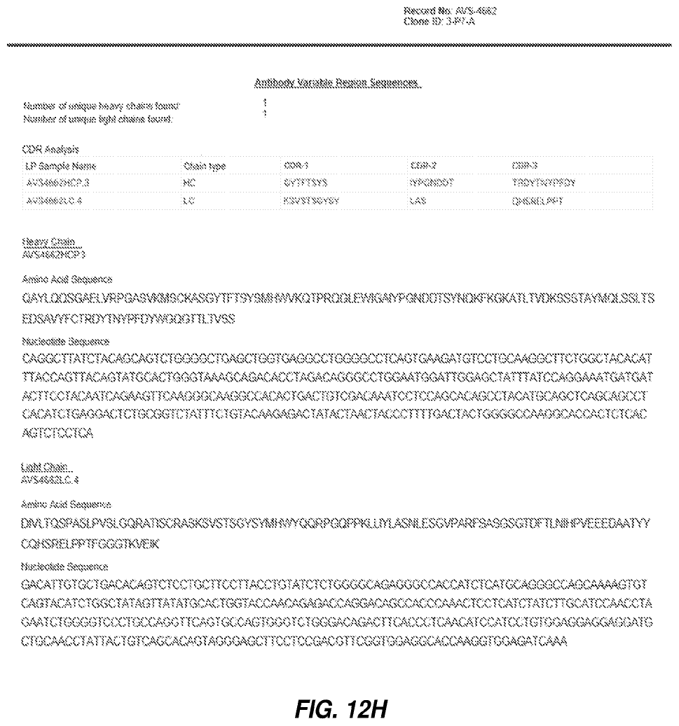

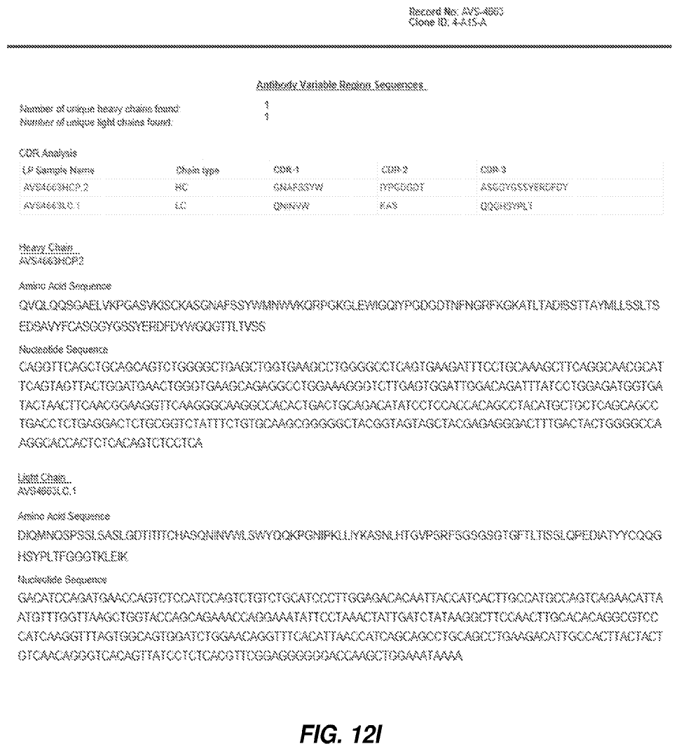

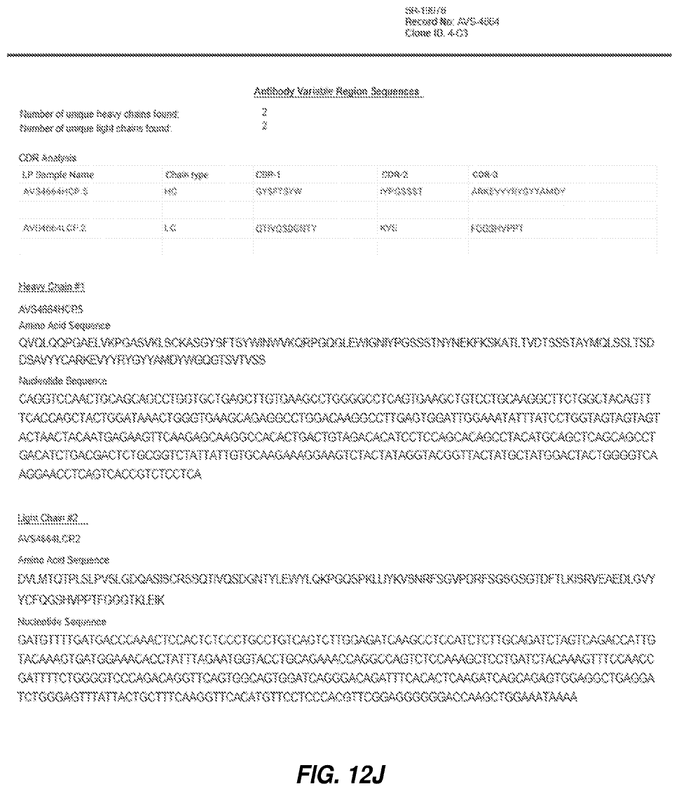

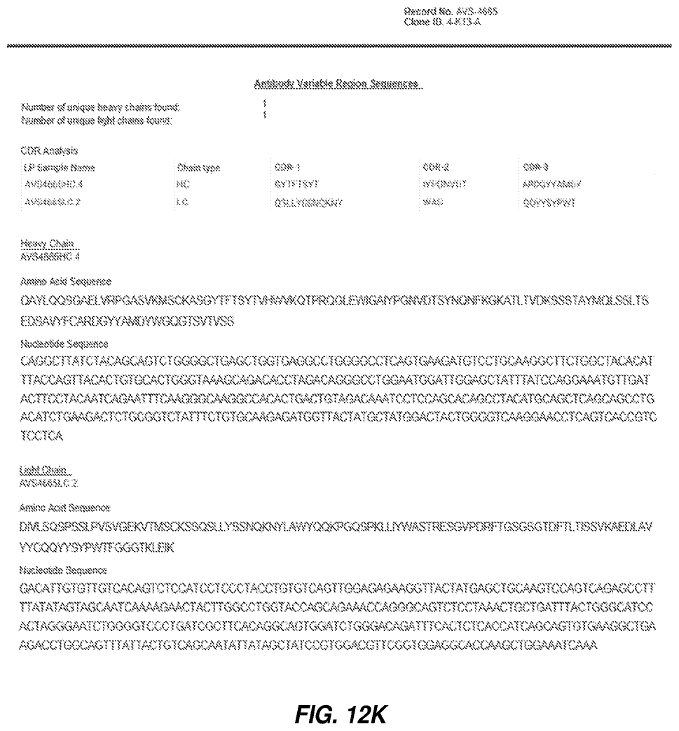

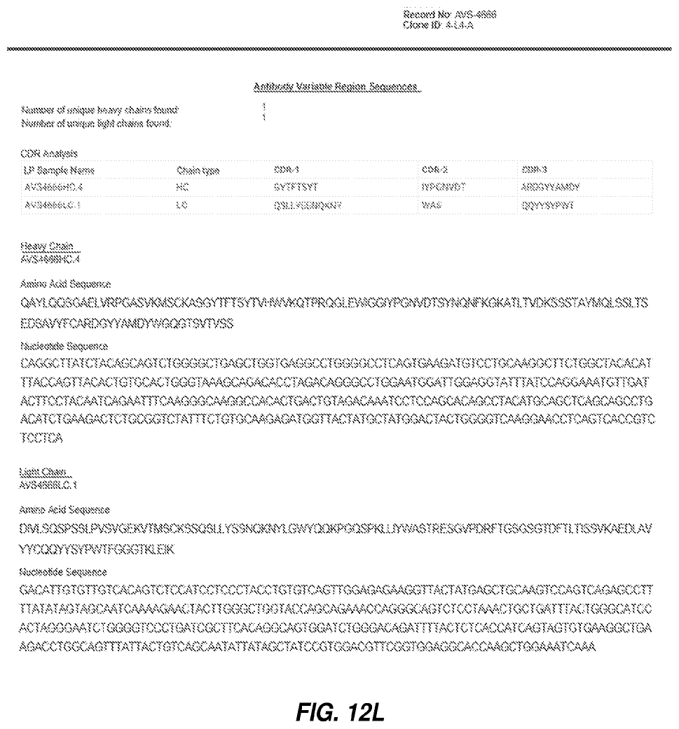

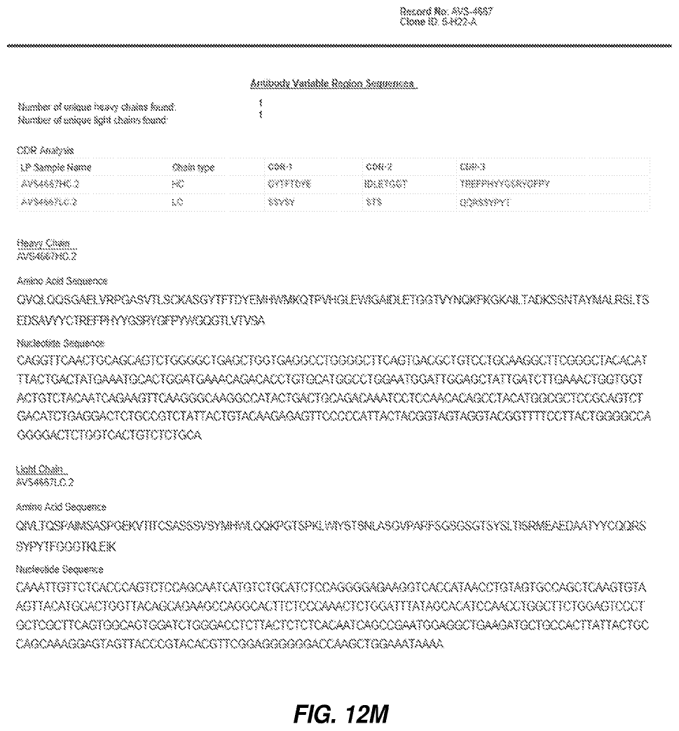

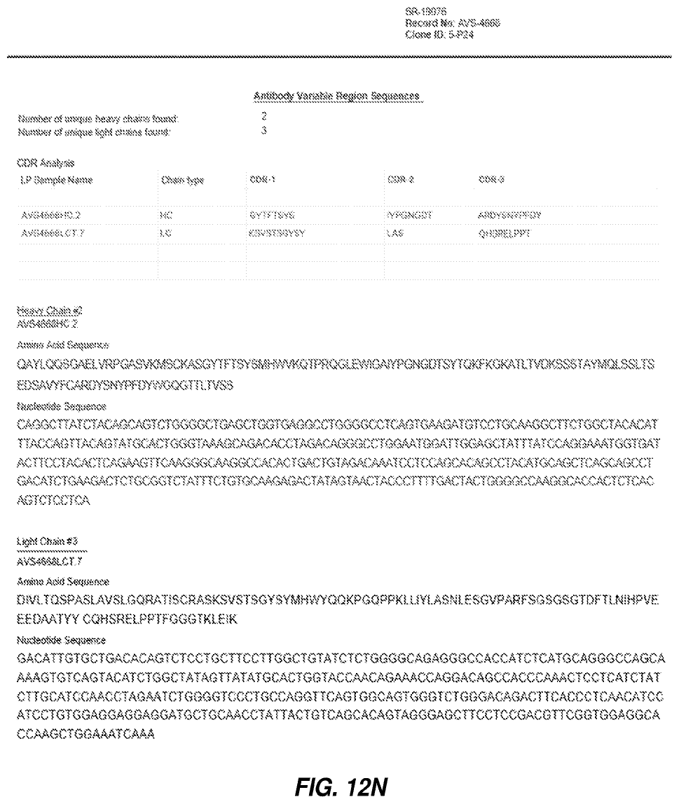

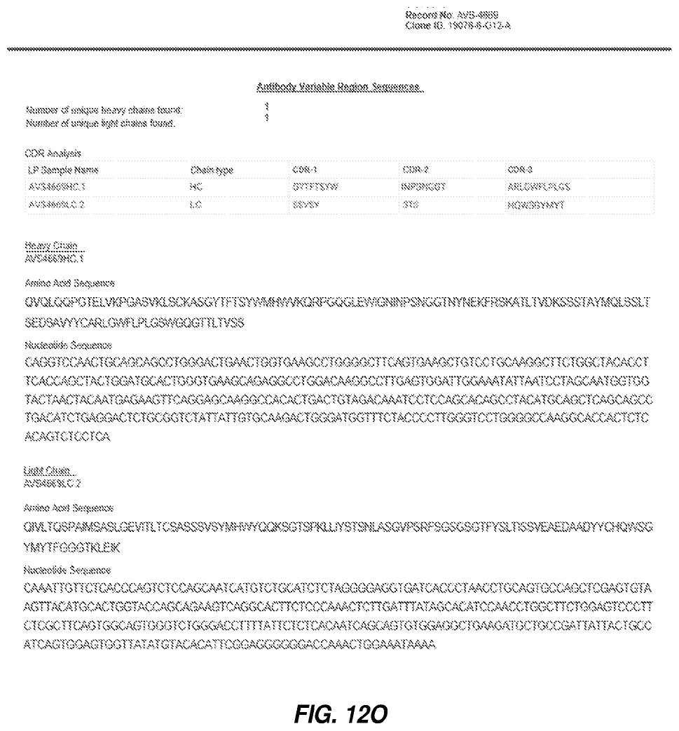

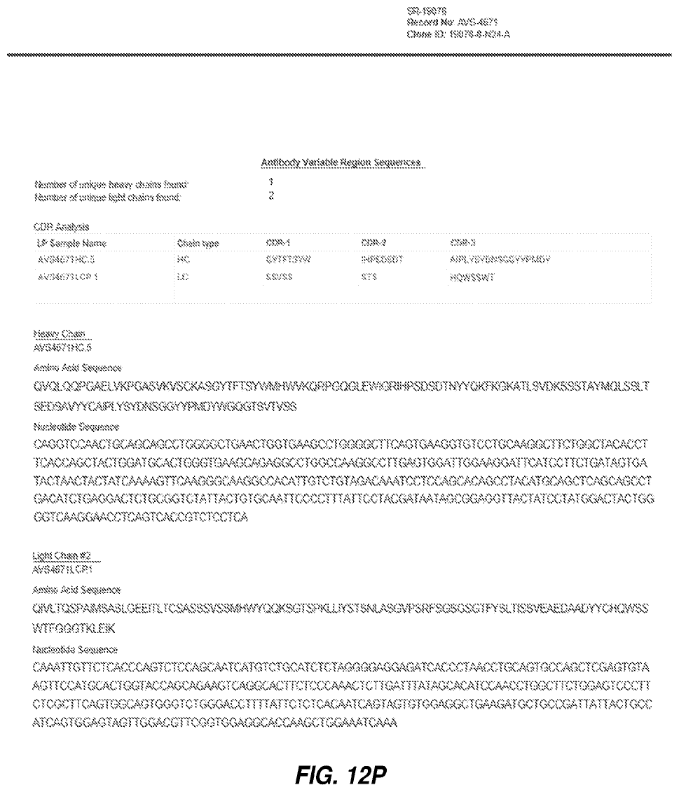

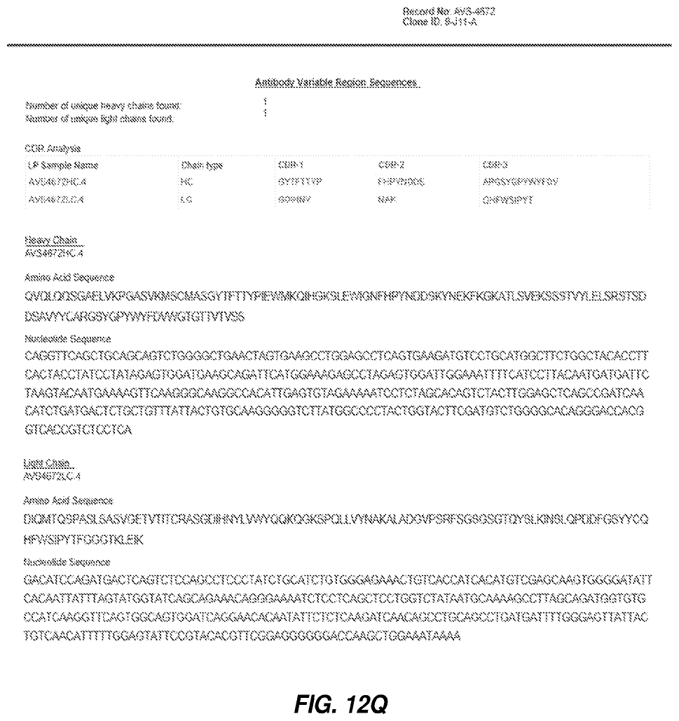

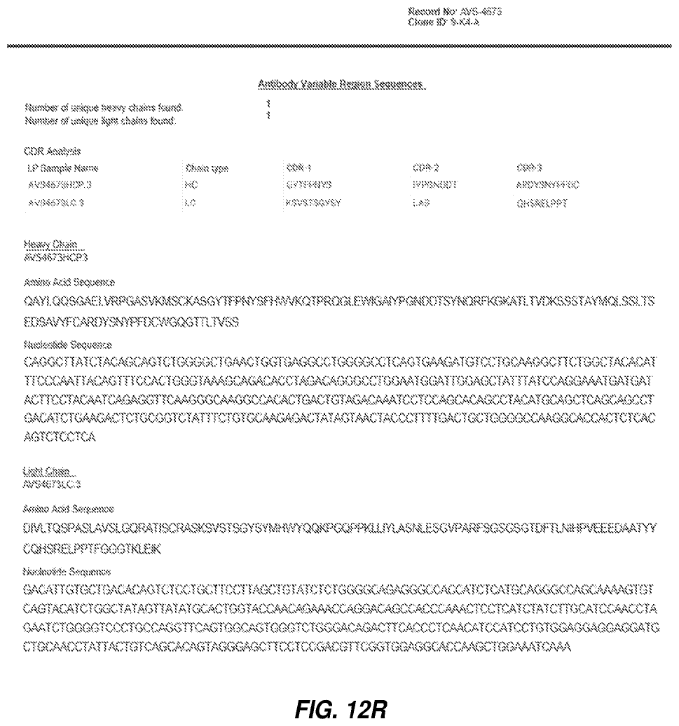

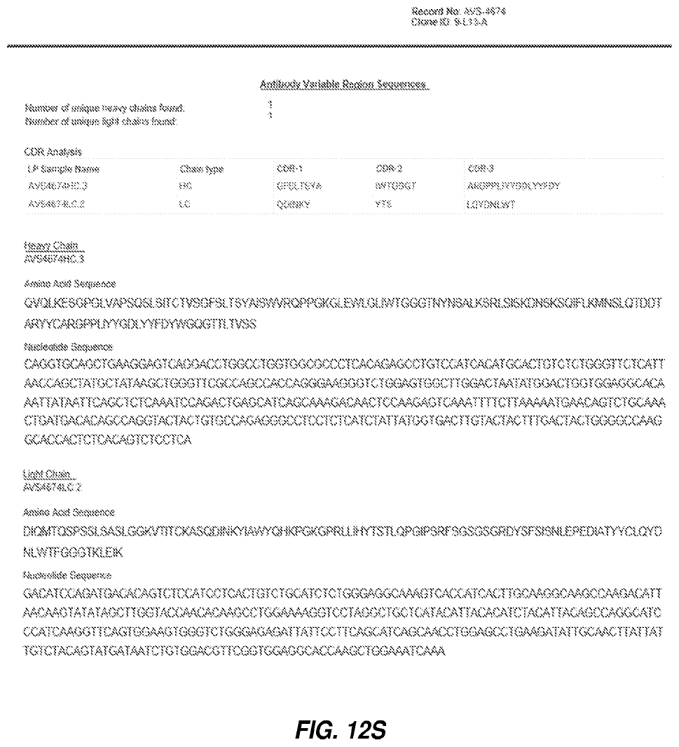

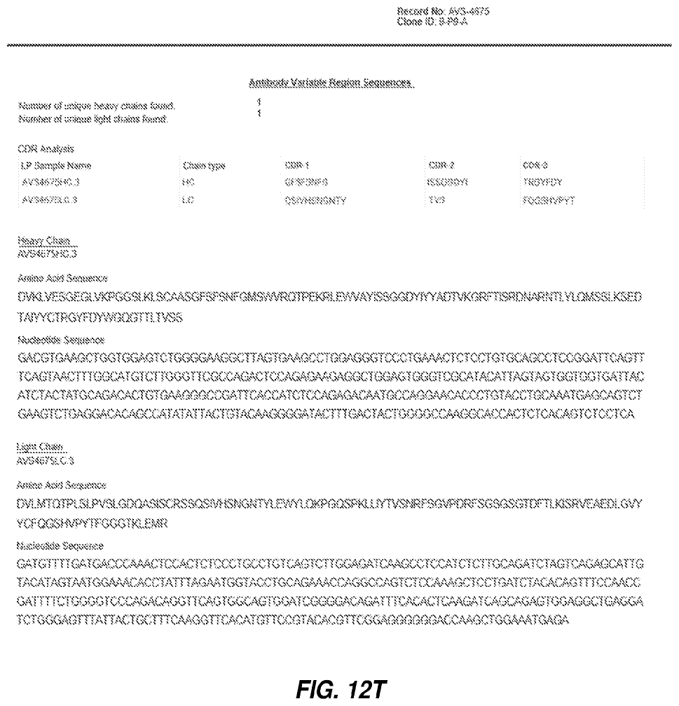

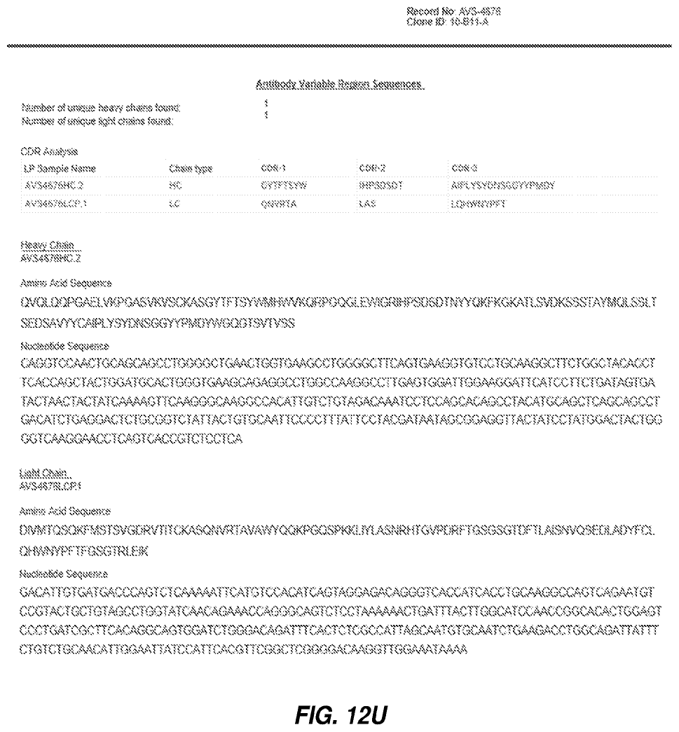

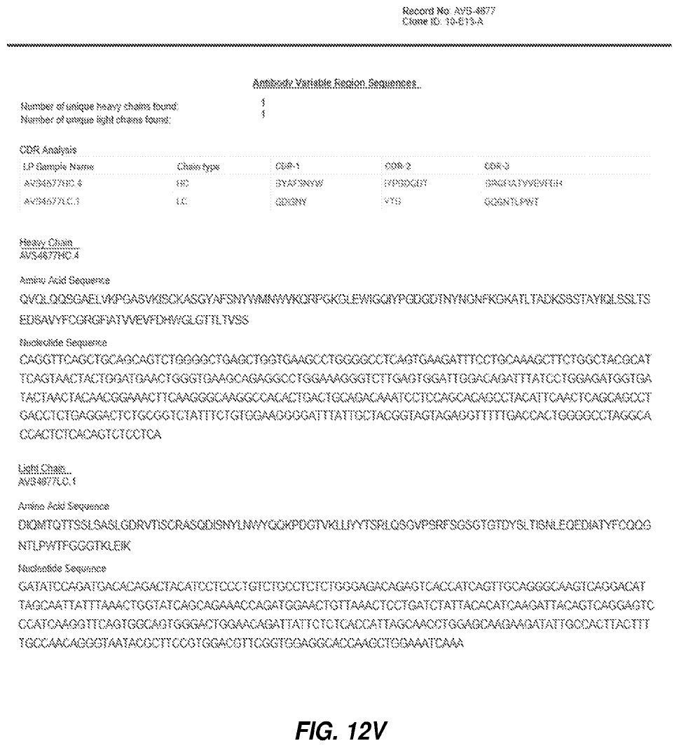

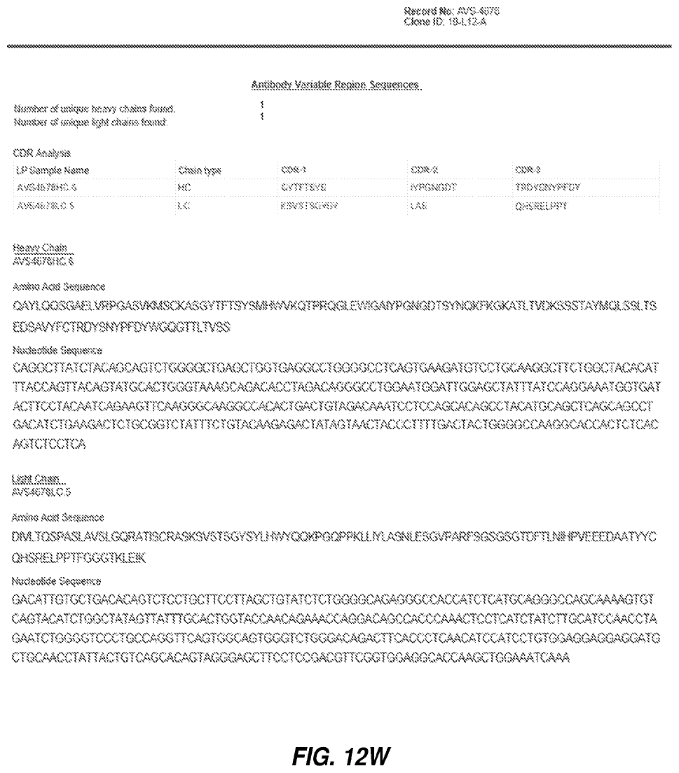

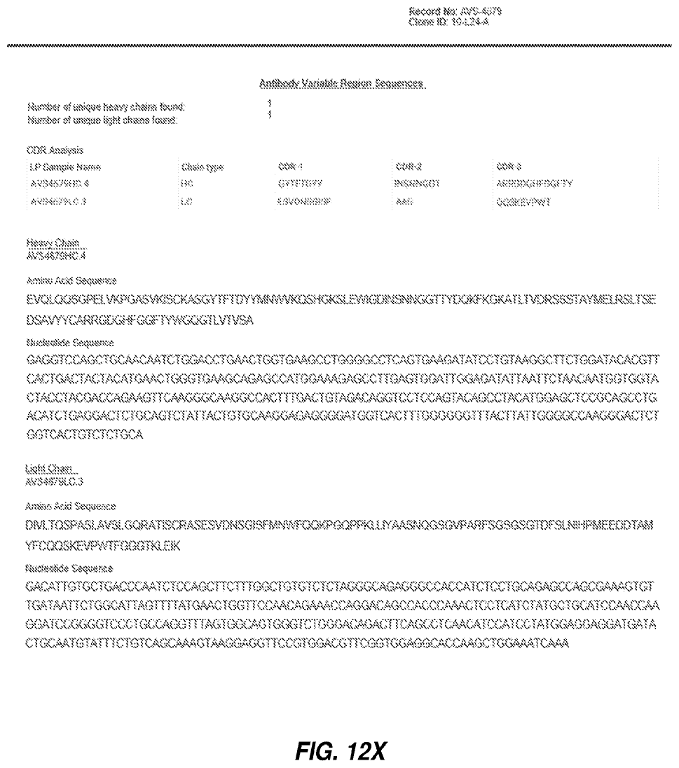

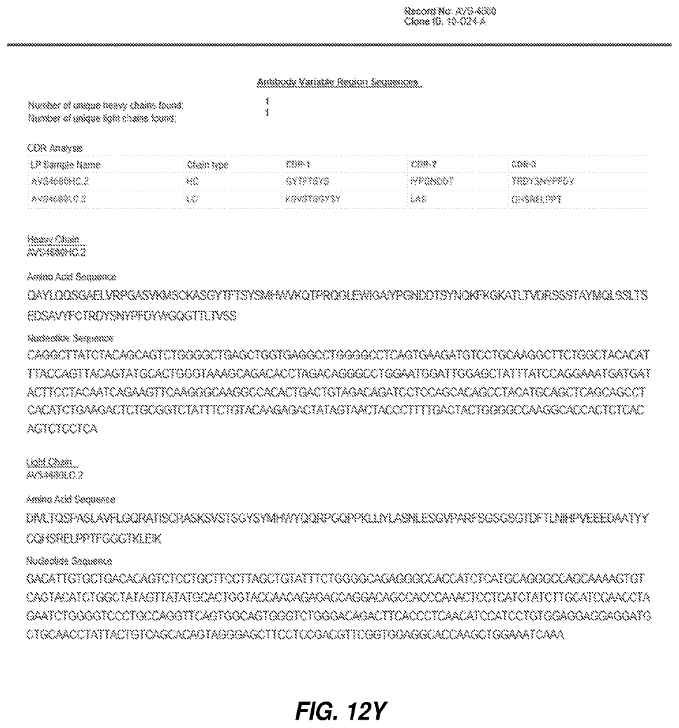

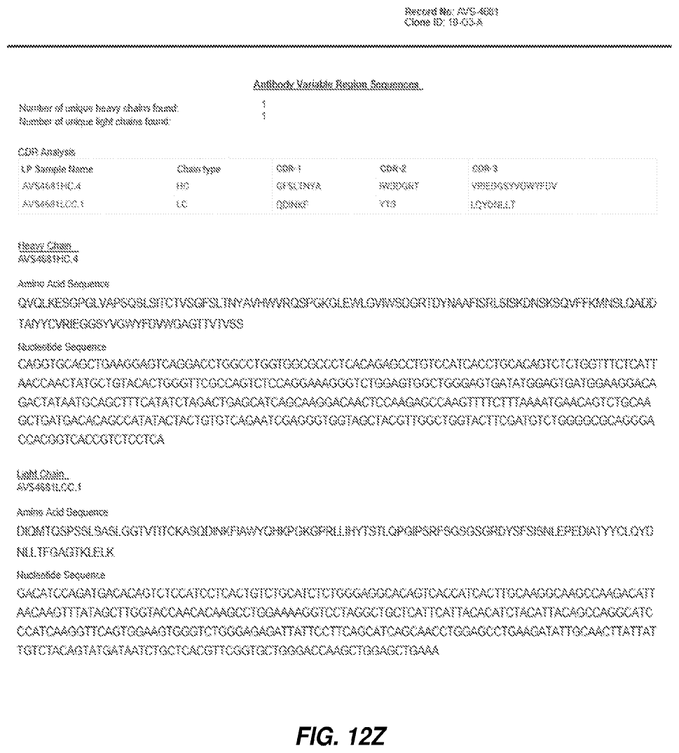

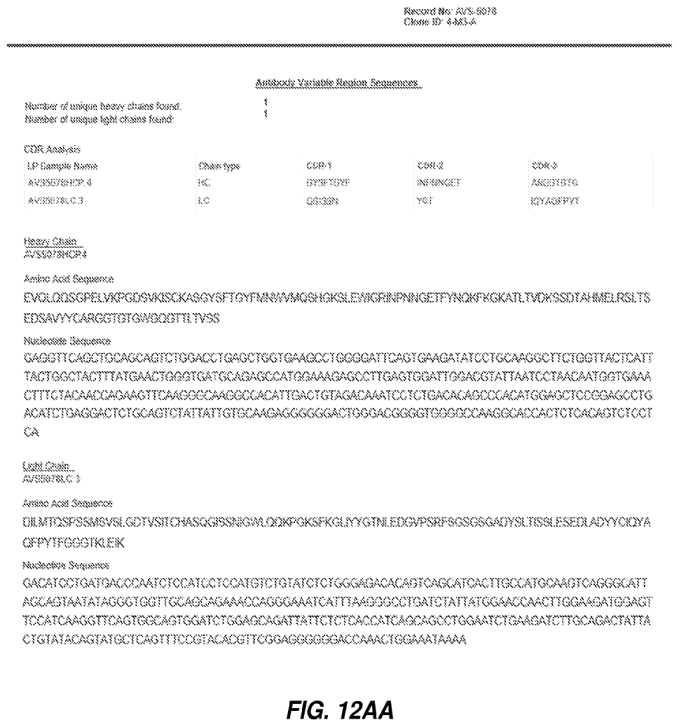

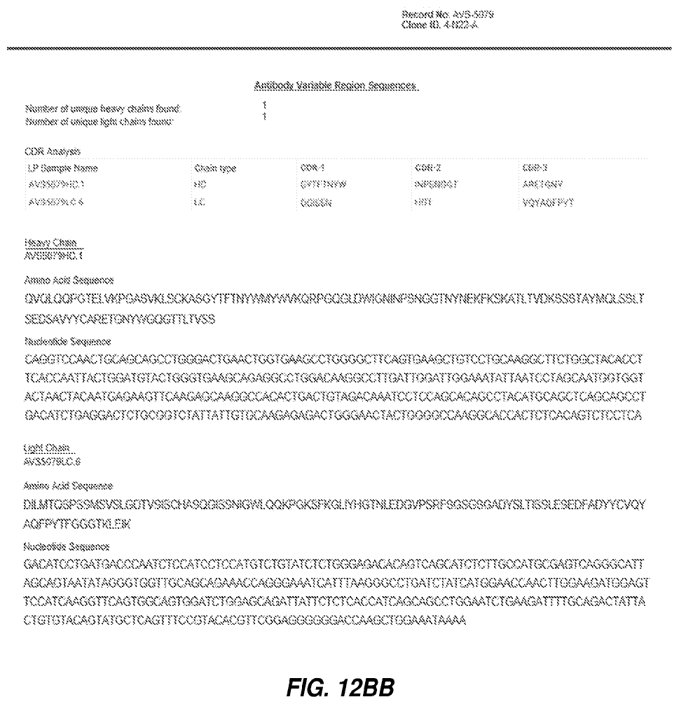

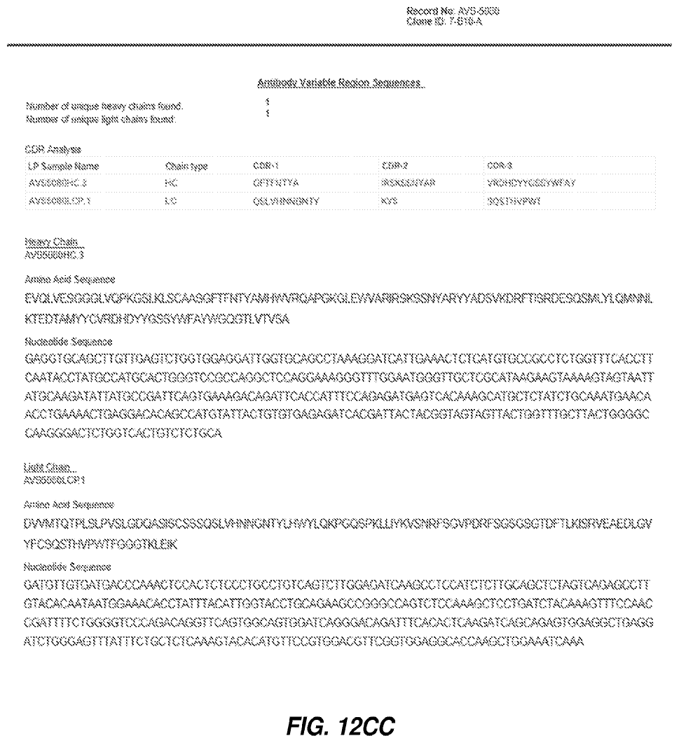

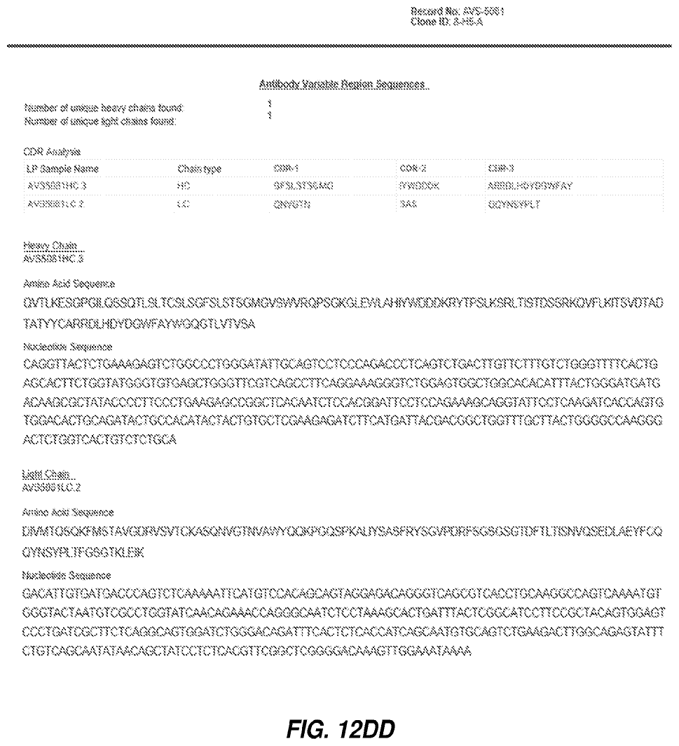

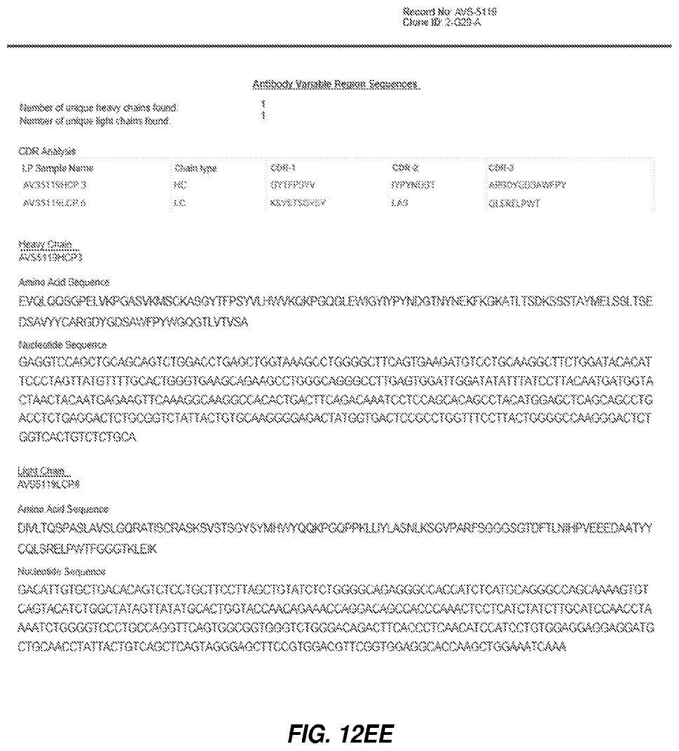

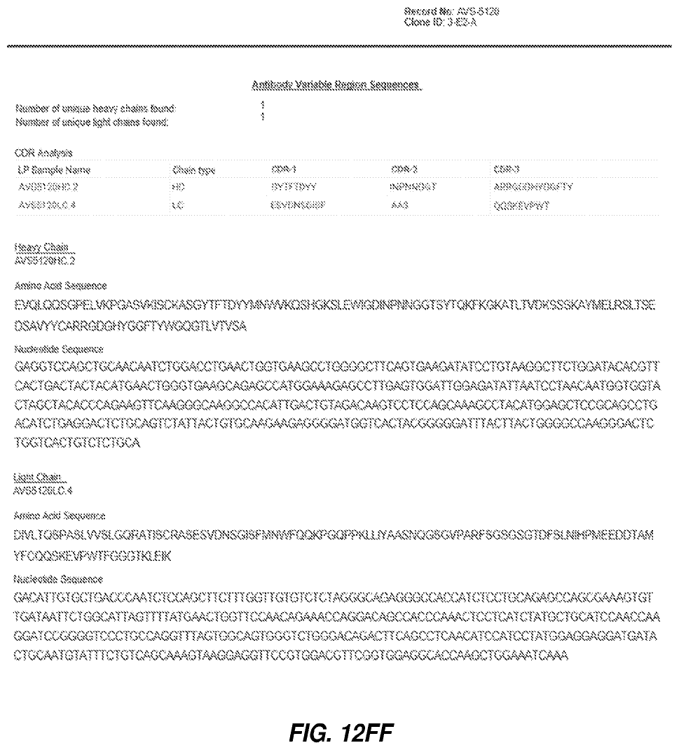

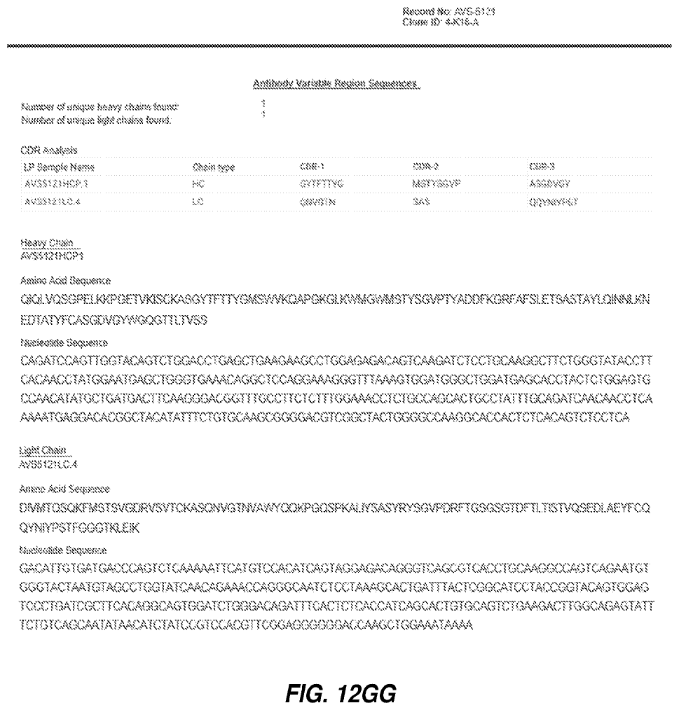

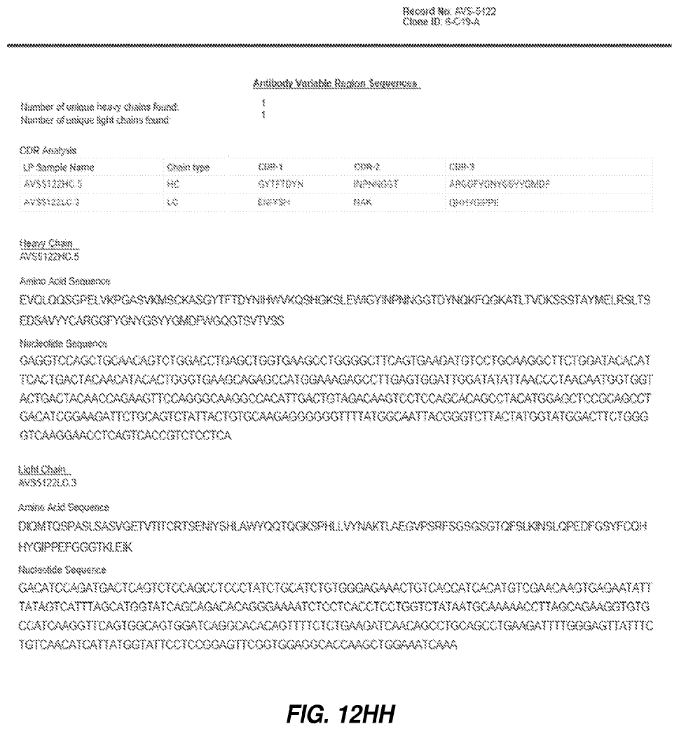

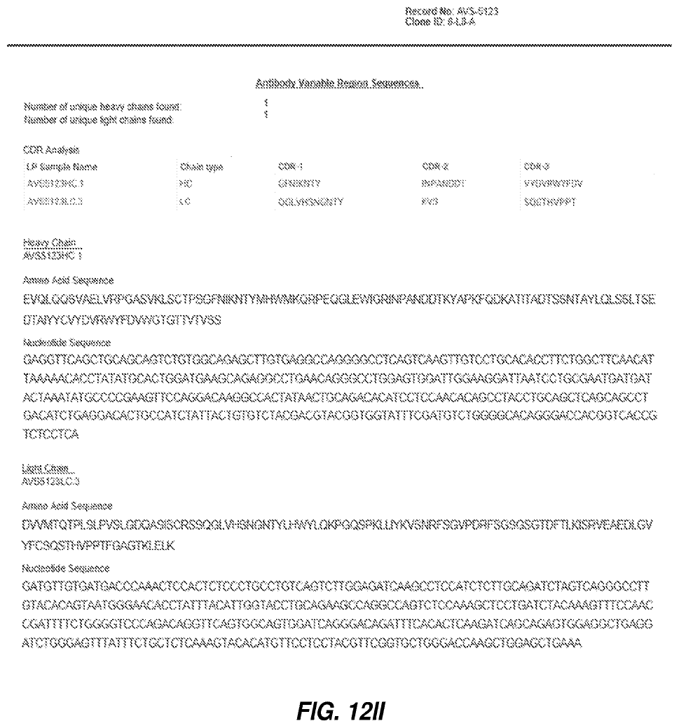

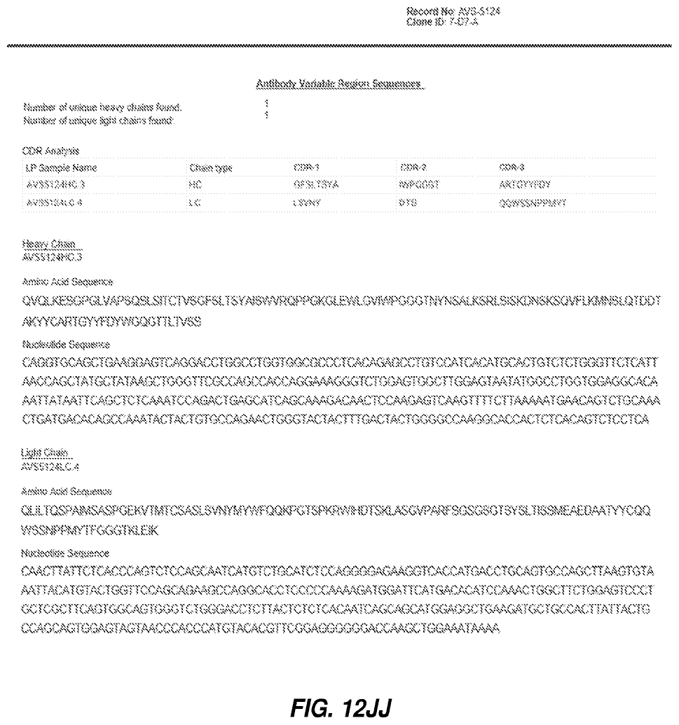

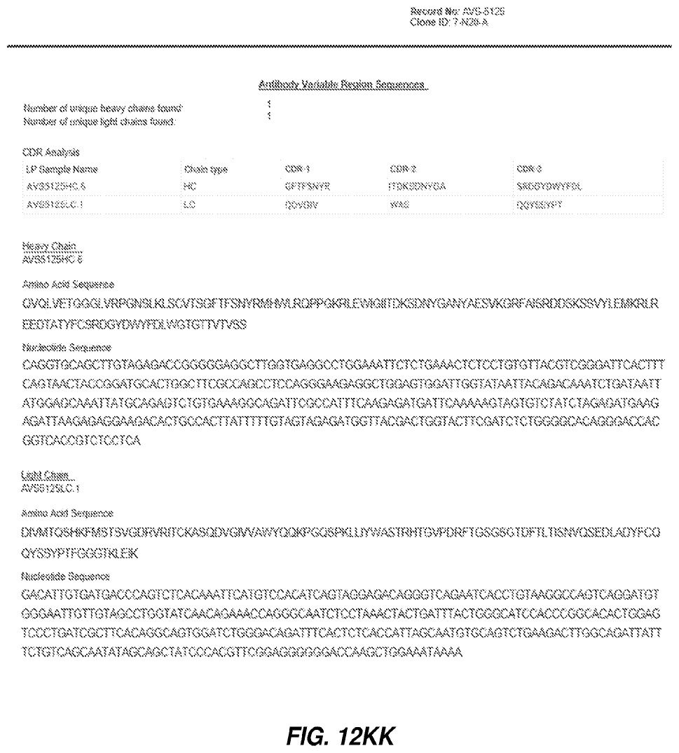

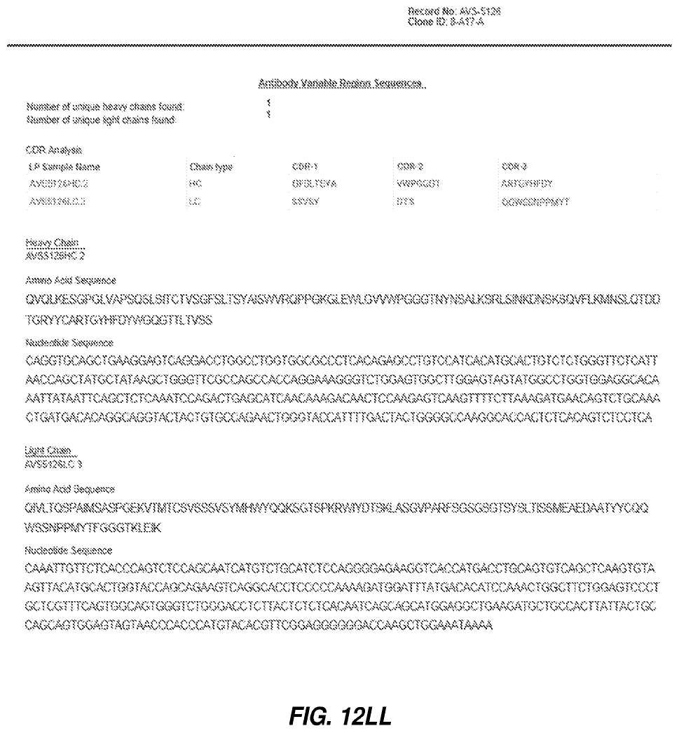

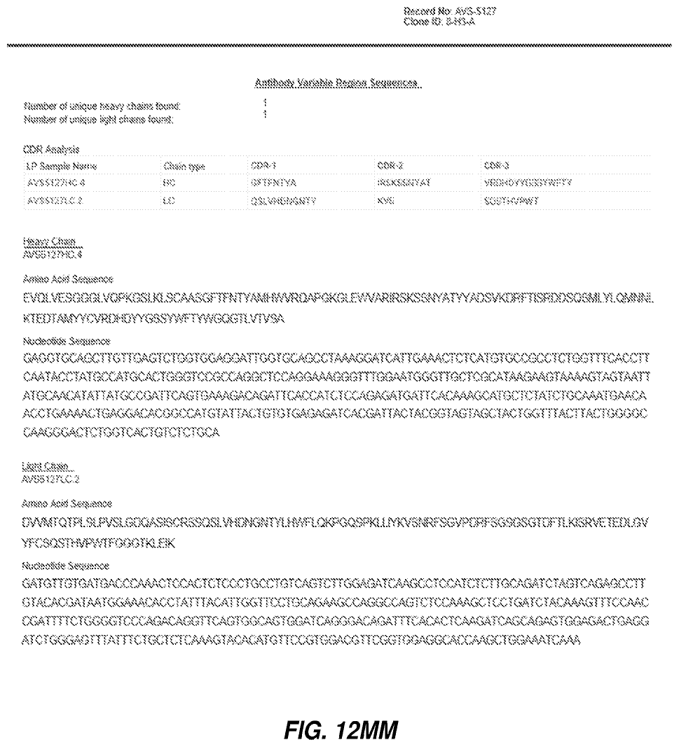

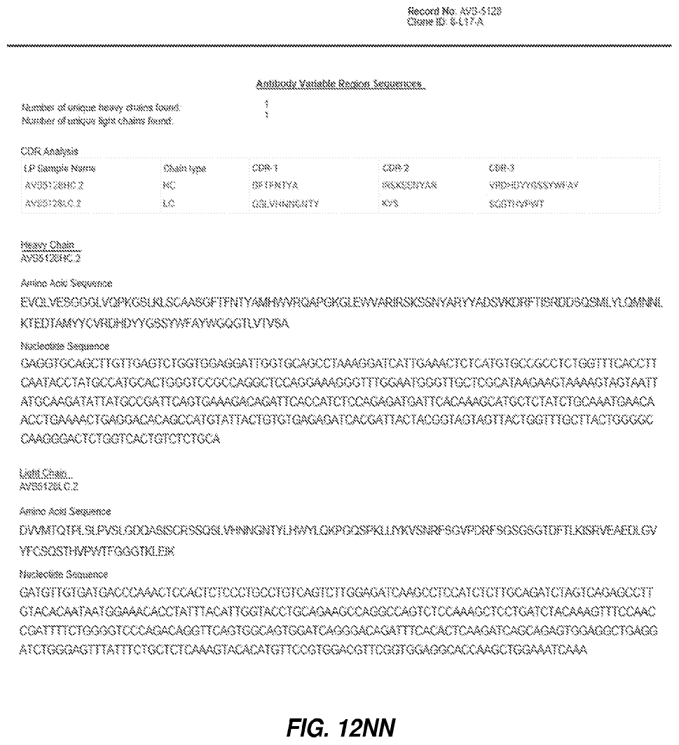

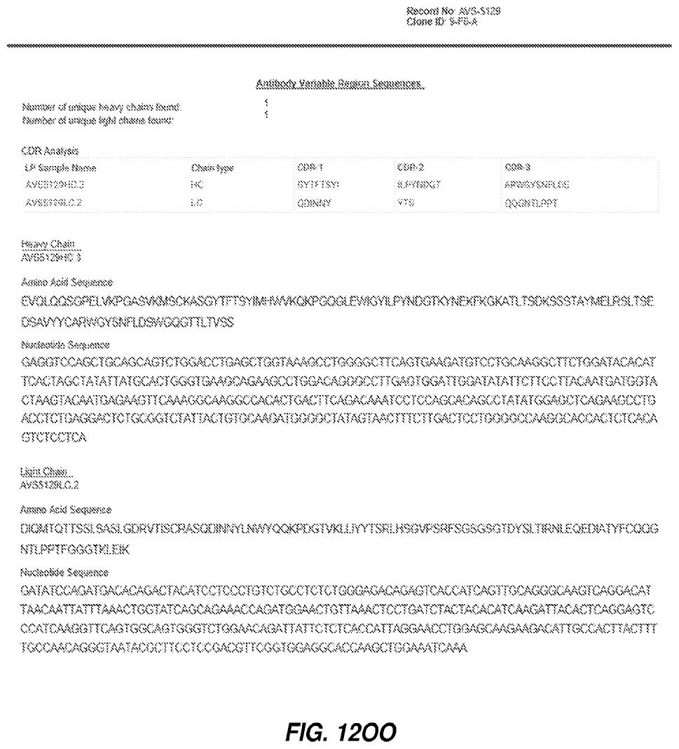

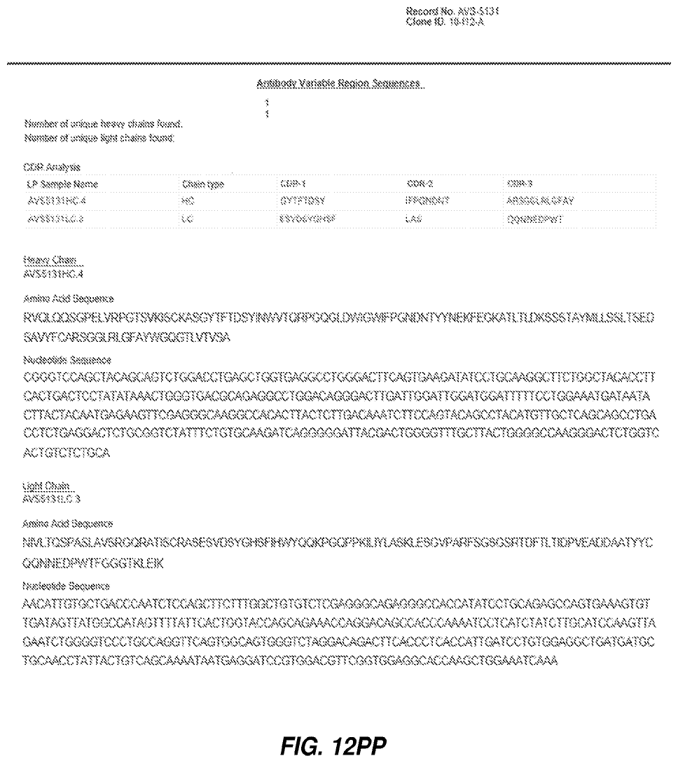

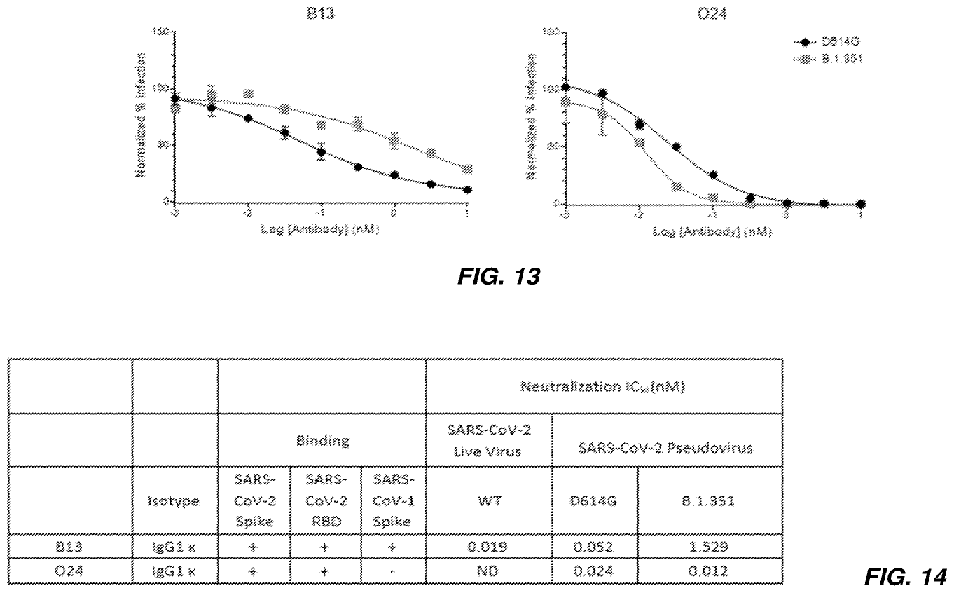

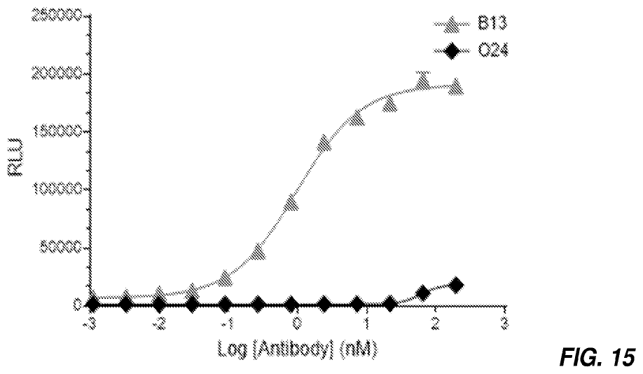

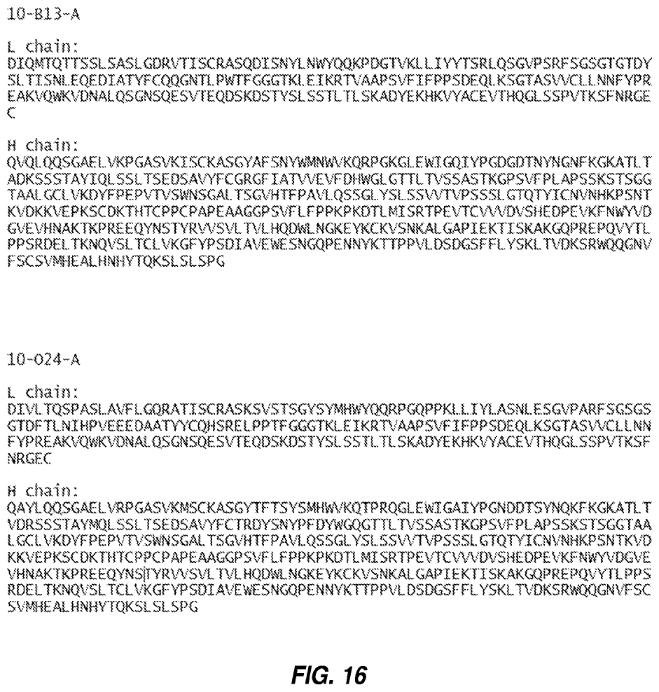

In FIG. 12A, the amino acid and encoding nucleotide sequences of 1-B11-A are shown for the heavy chain variable domain (SEQ ID NOS: 2 and 3, respectively) and the light chain variable domain (SEQ ID NOS: 4 and 5, respectively), and the amino acid sequences of the HC-CDR1 (SEQ ID NO: 6), HC-CDR2 (SEQ ID NO: 7), HC-CDR3 (SEQ ID NO: 8), LC-CDR1 (SEQ ID NO: 9), LC-CDR2 (SEQ ID NO: 10), and LC-CDR3 (SEQ ID NO: 11) are also shown.

In FIG. 12B, the amino acid and encoding nucleotide sequences of 1-L10-A are shown for the heavy chain variable domain (SEQ ID NOS: 12 and 13, respectively) and the light chain variable domain (SEQ ID NOS: 14 and 15, respectively), and the amino acid sequences of the HC-CDR1 (SEQ ID NO: 16), HC-CDR2 (SEQ ID NO: 17), HC-CDR3 (SEQ ID NO: 18), LC-CDR1 (SEQ ID NO: 19), LC-CDR2 (SEQ ID NO: 20), and LC-CDR3 (SEQ ID NO: 21) are also shown.

In FIG. 12C, the amino acid and encoding nucleotide sequences of 2-H7-A are shown for the heavy chain variable domain (SEQ ID NOS: 22 and 23, respectively) and the light chain variable domain (SEQ ID NOS: 24 and 25, respectively), and the amino acid sequences of the HC-CDR1 (SEQ ID NO: 26), HC-CDR2 (SEQ ID NO: 27), HC-CDR3 (SEQ ID NO: 28), LC-CDR1 (SEQ ID NO: 29), LC-CDR2 (SEQ ID NO: 30), and LC-CDR3 (SEQ ID NO: 31) are also shown.

In FIG. 12D, the amino acid and encoding nucleotide sequences of 2-J9-A are shown for the heavy chain variable domain (SEQ ID NOS: 32 and 33, respectively) and the light chain variable domain (SEQ ID NOS: 34 and 35, respectively), and the amino acid sequences of the HC-CDR1 (SEQ ID NO: 36), HC-CDR2 (SEQ ID NO: 37), HC-CDR3 (SEQ ID NO: 38), LC-CDR1 (SEQ ID NO: 39), LC-CDR2 (SEQ ID NO: 40), and LC-CDR3 (SEQ ID NO: 41) are also shown.

In FIG. 12E, the amino acid and encoding nucleotide sequences of 2-O12-A are shown for the heavy chain variable domain (SEQ ID NOS: 42 and 43, respectively) and the light chain variable domain (SEQ ID NOS: 44 and 45, respectively), and the amino acid sequences of the HC-CDR1 (SEQ ID NO: 46), HC-CDR2 (SEQ ID NO: 47), HC-CDR3 (SEQ ID NO: 48), LC-CDR1 (SEQ ID NO: 49), LC-CDR2 (SEQ ID NO: 50), and LC-CDR3 (SEQ ID NO: 51) are also shown.

In FIG. 12F, the amino acid and encoding nucleotide sequences of 2-P2-A are shown for the heavy chain variable domain (SEQ ID NOS: 52 and 53, respectively) and the light chain variable domain (SEQ ID NOS: 54 and 55, respectively), and the amino acid sequences of the HC-CDR1 (SEQ ID NO: 56), HC-CDR2 (SEQ ID NO: 57), HC-CDR3 (SEQ ID NO: 58), LC-CDR1 (SEQ ID NO: 59), LC-CDR2 (SEQ ID NO: 60), and LC-CDR3 (SEQ ID NO: 61) are also shown.

In FIG. 12G, the amino acid and encoding nucleotide sequences of 3-E13-A are shown for the heavy chain variable domain (SEQ ID NOS: 62 and 63, respectively) and the light chain variable domain (SEQ ID NOS: 64 and 65, respectively), and the amino acid sequences of the HC-CDR1 (SEQ ID NO: 66), HC-CDR2 (SEQ ID NO: 67), HC-CDR3 (SEQ ID NO: 68), LC-CDR1 (SEQ ID NO: 69), LC-CDR2 (SEQ ID NO: 70), and LC-CDR3 (SEQ ID NO: 71) are also shown.

In FIG. 12H, the amino acid and encoding nucleotide sequences of 3-P7-A are shown for the heavy chain variable domain (SEQ ID NOS: 72 and 73, respectively) and the light chain variable domain (SEQ ID NOS: 74 and 75, respectively), and the amino acid sequences of the HC-CDR1 (SEQ ID NO: 76), HC-CDR2 (SEQ ID NO: 77), HC-CDR3 (SEQ ID NO: 78), LC-CDR1 (SEQ ID NO: 79), LC-CDR2 (SEQ ID NO: 80), and LC-CDR3 (SEQ ID NO: 81) are also shown.

In FIG. 12I, the amino acid and encoding nucleotide sequences of 4-A15-A are shown for the heavy chain variable domain (SEQ ID NOS: 82 and 83, respectively) and the light chain variable domain (SEQ ID NOS: 84 and 85, respectively), and the amino acid sequences of the HC-CDR1 (SEQ ID NO: 86), HC-CDR2 (SEQ ID NO: 87), HC-CDR3 (SEQ ID NO: 88), LC-CDR1 (SEQ ID NO: 89), LC-CDR2 (SEQ ID NO: 90), and LC-CDR3 (SEQ ID NO: 91) are also shown.

In FIG. 12J, the amino acid and encoding nucleotide sequences of 4-C3-A are shown for the heavy chain variable domain (SEQ ID NOS: 92 and 93, respectively) and the light chain variable domain (SEQ ID NOS: 94 and 95, respectively), and the amino acid sequences of the HC-CDR1 (SEQ ID NO: 96), HC-CDR2 (SEQ ID NO: 97), HC-CDR3 (SEQ ID NO: 98), LC-CDR1 (SEQ ID NO: 99), LC-CDR2 (SEQ ID NO: 100), and LC-CDR3 (SEQ ID NO: 101) are also shown

In FIG. 12K, the amino acid and encoding nucleotide sequences of 4-K13-A are shown for the heavy chain variable domain (SEQ ID NOS: 102 and 103, respectively) and the light chain variable domain (SEQ ID NOS: 104 and 105, respectively), and the amino acid sequences of the HC-CDR1 (SEQ ID NO: 106), HC-CDR2 (SEQ ID NO: 107), HC-CDR3 (SEQ ID NO: 108), LC-CDR1 (SEQ ID NO: 109), LC-CDR2 (SEQ ID NO: 110), and LC-CDR3 (SEQ ID NO: 111) are also shown.

In FIG. 12L, the amino acid and encoding nucleotide sequences of 4-L4-A are shown for the heavy chain variable domain (SEQ ID NOS: 112 and 113, respectively) and the light chain variable domain (SEQ ID NOS: 114 and 115, respectively), and the amino acid sequences of the HC-CDR1 (SEQ ID NO: 116), HC-CDR2 (SEQ ID NO: 117), HC-CDR3 (SEQ ID NO: 118), LC-CDR1 (SEQ ID NO: 119), LC-CDR2 (SEQ ID NO: 120), and LC-CDR3 (SEQ ID NO: 121) are also shown.

In FIG. 12M, the amino acid and encoding nucleotide sequences of 5-H22-A are shown for the heavy chain variable domain (SEQ ID NOS: 122 and 123, respectively) and the light chain variable domain (SEQ ID NOS: 124 and 125, respectively), and the amino acid sequences of the HC-CDR1 (SEQ ID NO: 126), HC-CDR2 (SEQ ID NO: 127), HC-CDR3 (SEQ ID NO: 128), LC-CDR1 (SEQ ID NO: 129), LC-CDR2 (SEQ ID NO: 130), and LC-CDR3 (SEQ ID NO: 131) are also shown.

In FIG. 12N, the amino acid and encoding nucleotide sequences of 5-P24-A are shown for the heavy chain variable domain (SEQ ID NOS: 132 and 133, respectively) and the light chain variable domain (SEQ ID NOS: 134 and 135, respectively), and the amino acid sequences of the HC-CDR1 (SEQ ID NO: 136), HC-CDR2 (SEQ ID NO: 137), HC-CDR3 (SEQ ID NO: 138), LC-CDR1 (SEQ ID NO: 139), LC-CDR2 (SEQ ID NO: 140), and LC-CDR3 (SEQ ID NO: 141) are also shown.

In FIG. 12O, the amino acid and encoding nucleotide sequences of 6-O12-A are shown for the heavy chain variable domain (SEQ ID NOS: 142 and 143, respectively) and the light chain variable domain (SEQ ID NOS: 144 and 145, respectively), and the amino acid sequences of the HC-CDR1 (SEQ ID NO: 146), HC-CDR2 (SEQ ID NO: 147), HC-CDR3 (SEQ ID NO: 148), LC-CDR1 (SEQ ID NO: 149), LC-CDR2 (SEQ ID NO: 150), and LC-CDR3 (SEQ ID NO: 151) are also shown.

In FIG. 12P, the amino acid and encoding nucleotide sequences of 8-N24-A are shown for the heavy chain variable domain (SEQ ID NOS: 152 and 153, respectively) and the light chain variable domain (SEQ ID NOS: 154 and 155, respectively), and the amino acid sequences of the HC-CDR1 (SEQ ID NO: 156), HC-CDR2 (SEQ ID NO: 157), HC-CDR3 (SEQ ID NO: 158), LC-CDR1 (SEQ ID NO: 159), LC-CDR2 (SEQ ID NO: 160), and LC-CDR3 (SEQ ID NO: 161) are also shown.

In FIG. 12Q the amino acid and encoding nucleotide sequences of 9-J11-A are shown for the heavy chain variable domain (SEQ ID NOS: 162 and 163, respectively) and the light chain variable domain (SEQ ID NOS: 164 and 165, respectively), and the amino acid sequences of the HC-CDR1 (SEQ ID NO: 166), HC-CDR2 (SEQ ID NO: 167), HC-CDR3 (SEQ ID NO: 168), LC-CDR1 (SEQ ID NO: 169), LC-CDR2 (SEQ ID NO: 170), and LC-CDR3 (SEQ ID NO: 171) are also shown.

In FIG. 12R, the amino acid and encoding nucleotide sequences of 9-K4-A are shown for the heavy chain variable domain (SEQ ID NOS: 172 and 173, respectively) and the light chain variable domain (SEQ ID NOS: 174 and 175, respectively), and the amino acid sequences of the HC-CDR1 (SEQ ID NO: 176), HC-CDR2 (SEQ ID NO: 177), HC-CDR3 (SEQ ID NO: 178), LC-CDR1 (SEQ ID NO: 179), LC-CDR2 (SEQ ID NO: 180), and LC-CDR3 (SEQ ID NO: 181) are also shown.

In FIG. 12S, the amino acid and encoding nucleotide sequences of 9-L13-A are shown for the heavy chain variable domain (SEQ ID NOS: 182 and 183, respectively) and the light chain variable domain (SEQ ID NOS: 184 and 185, respectively), and the amino acid sequences of the HC-CDR1 (SEQ ID NO: 186), HC-CDR2 (SEQ ID NO: 187), HC-CDR3 (SEQ ID NO: 188), LC-CDR1 (SEQ ID NO: 189), LC-CDR2 (SEQ ID NO: 190), and LC-CDR3 (SEQ ID NO: 191) are also shown.

In FIG. 12T, the amino acid and encoding nucleotide sequences of 9-P9-A are shown for the heavy chain variable domain (SEQ ID NOS: 192 and 193, respectively) and the light chain variable domain (SEQ ID NOS: 194 and 195, respectively), and the amino acid sequences of the HC-CDR1 (SEQ ID NO: 196), HC-CDR2 (SEQ ID NO: 197), HC-CDR3 (SEQ ID NO: 198), LC-CDR1 (SEQ ID NO: 199), LC-CDR2 (SEQ ID NO: 200), and LC-CDR3 (SEQ ID NO: 201) are also shown.

In FIG. 12U, the amino acid and encoding nucleotide sequences of 10-B11-A are shown for the heavy chain variable domain (SEQ ID NOS: 202 and 203, respectively) and the light chain variable domain (SEQ ID NOS: 204 and 205, respectively), and the amino acid sequences of the HC-CDR1 (SEQ ID NO: 206), HC-CDR2 (SEQ ID NO: 207), HC-CDR3 (SEQ ID NO: 208), LC-CDR1 (SEQ ID NO: 209), LC-CDR2 (SEQ ID NO: 210), and LC-CDR3 (SEQ ID NO: 211) are also shown.

In FIG. 12V, the amino acid and encoding nucleotide sequences of 10-B13-A are shown for the heavy chain variable domain (SEQ ID NOS: 212 and 213, respectively) and the light chain variable domain (SEQ ID NOS: 214 and 215, respectively), and the amino acid sequences of the HC-CDR1 (SEQ ID NO: 216), HC-CDR2 (SEQ ID NO: 217), HC-CDR3 (SEQ ID NO: 218), LC-CDR1 (SEQ ID NO: 219), LC-CDR2 (SEQ ID NO: 220), and LC-CDR3 (SEQ ID NO: 221) are also shown.

In FIG. 12W, the amino acid and encoding nucleotide sequences of 10-L12-A are shown for the heavy chain variable domain (SEQ ID NOS: 222 and 223, respectively) and the light chain variable domain (SEQ ID NOS: 224 and 225, respectively), and the amino acid sequences of the HC-CDR1 (SEQ ID NO: 226), HC-CDR2 (SEQ ID NO: 227), HC-CDR3 (SEQ ID NO: 228), LC-CDR1 (SEQ ID NO: 229), LC-CDR2 (SEQ ID NO: 230), and LC-CDR3 (SEQ ID NO: 231) are also shown.

In FIG. 12X, the amino acid and encoding nucleotide sequences of 10-L24-A are shown for the heavy chain variable domain (SEQ ID NOS: 232 and 233, respectively) and the light chain variable domain (SEQ ID NOS: 234 and 235, respectively), and the amino acid sequences of the HC-CDR1 (SEQ ID NO: 236), HC-CDR2 (SEQ ID NO: 237), HC-CDR3 (SEQ ID NO: 238), LC-CDR1 (SEQ ID NO: 239), LC-CDR2 (SEQ ID NO: 240), and LC-CDR3 (SEQ ID NO: 241) are also shown.

In FIG. 12Y, the amino acid and encoding nucleotide sequences of 10-O24-A are shown for the heavy chain variable domain (SEQ ID NOS: 242 and 243, respectively) and the light chain variable domain (SEQ ID NOS: 244 and 245, respectively), and the amino acid sequences of the HC-CDR1 (SEQ ID NO: 246), HC-CDR2 (SEQ ID NO: 247), HC-CDR3 (SEQ ID NO: 248), LC-CDR1 (SEQ ID NO: 249), LC-CDR2 (SEQ ID NO: 250), and LC-CDR3 (SEQ ID NO: 251) are also shown.

In FIG. 12Z, the amino acid and encoding nucleotide sequences of 10-O3-A are shown for the heavy chain variable domain (SEQ ID NOS: 252 and 253, respectively) and the light chain variable domain (SEQ ID NOS: 254 and 255, respectively), and the amino acid sequences of the HC-CDR1 (SEQ ID NO: 256), HC-CDR2 (SEQ ID NO: 257), HC-CDR3 (SEQ ID NO: 258), LC-CDR1 (SEQ ID NO: 259), LC-CDR2 (SEQ ID NO: 260), and LC-CDR3 (SEQ ID NO: 261) are also shown.

In FIG. 12AA, the amino acid and encoding nucleotide sequences of 4-M3-A are shown for the heavy chain variable domain (SEQ ID NOS: 262 and 263, respectively) and the light chain variable domain (SEQ ID NOS: 264 and 265, respectively), and the amino acid sequences of the HC-CDR1 (SEQ ID NO: 266), HC-CDR2 (SEQ ID NO: 267), HC-CDR3 (SEQ ID NO: 268), LC-CDR1 (SEQ ID NO: 269), LC-CDR2 (SEQ ID NO: 270), and LC-CDR3 (SEQ ID NO: 271) are also shown.

In FIG. 12BB, the amino acid and encoding nucleotide sequences of 4-N22-A are shown for the heavy chain variable domain (SEQ ID NOS: 272 and 273, respectively) and the light chain variable domain (SEQ ID NOS: 274 and 275, respectively), and the amino acid sequences of the HC-CDR1 (SEQ ID NO: 276), HC-CDR2 (SEQ ID NO: 277), HC-CDR3 (SEQ ID NO: 278), LC-CDR1 (SEQ ID NO: 279), LC-CDR2 (SEQ ID NO: 280), and LC-CDR3 (SEQ ID NO: 281) are also shown.

In FIG. 12CC, the amino acid and encoding nucleotide sequences of 7-B10-A are shown for the heavy chain variable domain (SEQ ID NOS: 282 and 283, respectively) and the light chain variable domain (SEQ ID NOS: 284 and 285, respectively), and the amino acid sequences of the HC-CDR1 (SEQ ID NO: 286), HC-CDR2 (SEQ ID NO: 287), HC-CDR3 (SEQ ID NO: 288), LC-CDR1 (SEQ ID NO: 289), LC-CDR2 (SEQ ID NO: 290), and LC-CDR3 (SEQ ID NO: 291) are also shown.

In FIG. 12DD, the amino acid and encoding nucleotide sequences of 8-H5-A are shown for the heavy chain variable domain (SEQ ID NOS: 292 and 293, respectively) and the light chain variable domain (SEQ ID NOS: 294 and 295, respectively), and the amino acid sequences of the HC-CDR1 (SEQ ID NO: 296), HC-CDR2 (SEQ ID NO: 297), HC-CDR3 (SEQ ID NO: 298), LC-CDR1 (SEQ ID NO: 299), LC-CDR2 (SEQ ID NO: 300), and LC-CDR3 (SEQ ID NO: 301) are also shown.

In FIG. 12EE, the amino acid and encoding nucleotide sequences of 2-G20-A are shown for the heavy chain variable domain (SEQ ID NOS: 302 and 303, respectively) and the light chain variable domain (SEQ ID NOS: 304 and 305, respectively), and the amino acid sequences of the HC-CDR1 (SEQ ID NO: 306), HC-CDR2 (SEQ ID NO: 307), HC-CDR3 (SEQ ID NO: 308), LC-CDR1 (SEQ ID NO: 309), LC-CDR2 (SEQ ID NO: 310), and LC-CDR3 (SEQ ID NO: 311) are also shown.

In FIG. 12FF, the amino acid and encoding nucleotide sequences of 3-E2-A are shown for the heavy chain variable domain (SEQ ID NOS: 312 and 313, respectively) and the light chain variable domain (SEQ ID NOS: 314 and 315, respectively), and the amino acid sequences of the HC-CDR1 (SEQ ID NO: 316), HC-CDR2 (SEQ ID NO: 317), HC-CDR3 (SEQ ID NO: 318), LC-CDR1 (SEQ ID NO: 319), LC-CDR2 (SEQ ID NO: 320), and LC-CDR3 (SEQ ID NO: 321) are also shown.

In FIG. 12GG, the amino acid and encoding nucleotide sequences of 4-K16-A are shown for the heavy chain variable domain (SEQ ID NOS: 322 and 323, respectively) and the light chain variable domain (SEQ ID NOS: 324 and 325, respectively), and the amino acid sequences of the HC-CDR1 (SEQ ID NO: 326), HC-CDR2 (SEQ ID NO: 327), HC-CDR3 (SEQ ID NO: 328), LC-CDR1 (SEQ ID NO: 329), LC-CDR2 (SEQ ID NO: 330), and LC-CDR3 (SEQ ID NO: 331) are also shown.

In FIG. 12HH, the amino acid and encoding nucleotide sequences of 6-C19-A are shown for the heavy chain variable domain (SEQ ID NOS: 332 and 333, respectively) and the light chain variable domain (SEQ ID NOS: 334 and 335, respectively), and the amino acid sequences of the HC-CDR1 (SEQ ID NO: 336), HC-CDR2 (SEQ ID NO: 337), HC-CDR3 (SEQ ID NO: 338), LC-CDR1 (SEQ ID NO: 339), LC-CDR2 (SEQ ID NO: 340), and LC-CDR3 (SEQ ID NO: 341) are also shown.

In FIG. 12II, the amino acid and encoding nucleotide sequences of 6-L8-A are shown for the heavy chain variable domain (SEQ ID NOS: 342 and 343, respectively) and the light chain variable domain (SEQ ID NOS: 344 and 345, respectively), and the amino acid sequences of the HC-CDR1 (SEQ ID NO: 346), HC-CDR2 (SEQ ID NO: 347), HC-CDR3 (SEQ ID NO: 348), LC-CDR1 (SEQ ID NO: 349), LC-CDR2 (SEQ ID NO: 350), and LC-CDR3 (SEQ ID NO: 351) are also shown.

In FIG. 12JJ, the amino acid and encoding nucleotide sequences of 7-D7-A are shown for the heavy chain variable domain (SEQ ID NOS: 352 and 353, respectively) and the light chain variable domain (SEQ ID NOS: 354 and 355, respectively), and the amino acid sequences of the HC-CDR1 (SEQ ID NO: 356), HC-CDR2 (SEQ ID NO: 357), HC-CDR3 (SEQ ID NO: 358), LC-CDR1 (SEQ ID NO: 359), LC-CDR2 (SEQ ID NO: 360), and LC-CDR3 (SEQ ID NO: 361) are also shown.

In FIG. 12KK, the amino acid and encoding nucleotide sequences of 7-N20-A are shown for the heavy chain variable domain (SEQ ID NOS: 362 and 363, respectively) and the light chain variable domain (SEQ ID NOS: 364 and 365, respectively), and the amino acid sequences of the HC-CDR1 (SEQ ID NO: 366), HC-CDR2 (SEQ ID NO: 367), HC-CDR3 (SEQ ID NO: 368), LC-CDR1 (SEQ ID NO: 369), LC-CDR2 (SEQ ID NO: 370), and LC-CDR3 (SEQ ID NO: 371) are also shown.

In FIG. 12LL, the amino acid and encoding nucleotide sequences of 8-A17-A are shown for the heavy chain variable domain (SEQ ID NOS: 372 and 373, respectively) and the light chain variable domain (SEQ ID NOS: 374 and 375, respectively), and the amino acid sequences of the HC-CDR1 (SEQ ID NO: 376), HC-CDR2 (SEQ ID NO: 377), HC-CDR3 (SEQ ID NO: 378), LC-CDR1 (SEQ ID NO: 379), LC-CDR2 (SEQ ID NO: 380), and LC-CDR3 (SEQ ID NO: 381) are also shown.

In FIG. 12MM, the amino acid and encoding nucleotide sequences of 8-H3-A are shown for the heavy chain variable domain (SEQ ID NOS: 382 and 383, respectively) and the light chain variable domain (SEQ ID NOS: 384 and 385, respectively), and the amino acid sequences of the HC-CDR1 (SEQ ID NO: 386), HC-CDR2 (SEQ ID NO: 387), HC-CDR3 (SEQ ID NO: 388), LC-CDR1 (SEQ ID NO: 389), LC-CDR2 (SEQ ID NO: 390), and LC-CDR3 (SEQ ID NO: 391) are also shown.

In FIG. 12NN, the amino acid and encoding nucleotide sequences of 8-L17-A are shown for the heavy chain variable domain (SEQ ID NOS: 392 and 393, respectively) and the light chain variable domain (SEQ ID NOS: 394 and 395, respectively), and the amino acid sequences of the HC-CDR1 (SEQ ID NO: 396), HC-CDR2 (SEQ ID NO: 397), HC-CDR3 (SEQ ID NO: 398), LC-CDR1 (SEQ ID NO: 399), LC-CDR2 (SEQ ID NO: 400), and LC-CDR3 (SEQ ID NO: 401) are also shown.

In FIG. 1200, the amino acid and encoding nucleotide sequences of 9-F6-A are shown for the heavy chain variable domain (SEQ ID NOS: 402 and 403, respectively) and the light chain variable domain (SEQ ID NOS: 404 and 405, respectively), and the amino acid sequences of the HC-CDR1 (SEQ ID NO: 406), HC-CDR2 (SEQ ID NO: 407), HC-CDR3 (SEQ ID NO: 408), LC-CDR1 (SEQ ID NO: 409), LC-CDR2 (SEQ ID NO: 410), and LC-CDR3 (SEQ ID NO: 411) are also shown.

In FIG. 12PP, the amino acid and encoding nucleotide sequences of 10-I12-A are shown for the heavy chain variable domain (SEQ ID NOS: 412 and 413, respectively) and the light chain variable domain (SEQ ID NOS: 414 and 415, respectively), and the amino acid sequences of the HC-CDR1 (SEQ ID NO: 416), HC-CDR2 (SEQ ID NO: 417), HC-CDR3 (SEQ ID NO: 418), LC-CDR1 (SEQ ID NO: 419), LC-CDR2 (SEQ ID NO: 420), and LC-CDR3 (SEQ ID NO: 421) are also shown.

FIG. 13 is a pair of graphs illustrating in vitro pseudovirus neutralization of SARS-CoV-2 D614G and B.1.351 variants using antibodies B13, also referred to as 10-B13-A (left), and O24, also referred to as 10-O24-A (right). Neutralization of luciferase-tagged pseudotyped SARS-CoV-2 D614G (circles) and B.1.351 variant (squares) 72 hours after inoculation is shown. Values plotted are means of two replicates (n=2), with error bars showing SD.

FIG. 14 is a table summarizing the selectivity and potency of SARS-CoV-2 monoclonal antibodies B13 and O24. WT=wild-type SARS-CoV-2; ND=No Data.

FIG. 15 is a graph illustrating SARS-CoV-1 spike protein binding cross-reactivity. Binding of purified mouse antibodies B13 (triangles) and O24 (diamonds) to SARS-CoV-1 spike protein as determined by ELISA is shown. The EC₅₀ for B13 binding is 0.96 nM. Values plotted are means of two replicates (n=2), with error bars showing range. RLU: relative luminescence signal.

FIG. 16 includes the amino acid sequences of B13 and O24 antibodies. The light and heavy chains of antibody B13 are SEQ ID NOS: 422 and 423, respectively. The light and heavy chains of antibody O24 are SEQ ID NOS: 424 and 425, respectively.

### DETAILED DESCRIPTION OF THE INVENTION

Before the invention is described in greater detail, it is to be understood that the invention is not limited to particular embodiments described herein as such embodiments may vary. It is also to be understood that the terminology used herein is for the purpose of describing particular embodiments only, and the terminology is not intended to be limiting. The scope of the invention will be limited only by the appended claims. Unless defined otherwise, all technical and scientific terms used herein have the same meaning as commonly understood by one of ordinary skill in the art to which this invention belongs. Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number, which, in the context in which it is presented, provides the substantial equivalent of the specifically recited number. All publications, patents, and patent applications cited in this specification are incorporated herein by reference to the same extent as if each individual publication, patent, or patent application were specifically and individually indicated to be incorporated by reference. Furthermore, each cited publication, patent, or patent application is incorporated herein by reference to disclose and describe the subject matter in connection with which the publications are cited. The citation of any publication is for its disclosure prior to the filing date and should not be construed as an admission that the invention described herein is not entitled to antedate such publication by virtue of prior invention. Further, the dates of publication provided might be different from the actual publication dates, which may need to be independently confirmed.

It is noted that the claims may be drafted to exclude any optional element. As such, this statement is intended to serve as antecedent basis for use of such exclusive terminology as “solely,” “only,” and the like in connection with the recitation of claim elements, or use of a “negative” limitation. As will be apparent to those of skill in the art upon reading this disclosure, each of the individual embodiments described and illustrated herein has discrete components and features which may be readily separated from or combined with the features of any of the other several embodiments without departing from the scope or spirit of the invention. Any recited method may be carried out in the order of events recited or in any other order that is logically possible. Although any methods and materials similar or equivalent to those described herein may also be used in the practice or testing of the invention, representative illustrative methods and materials are now described.

In describing the present invention, the following terms will be employed, and are intended to be defined as indicated below.

### A. Definitions

In order that the invention may be more completely understood, several definitions are set forth below. Such definitions are meant to encompass grammatical equivalents.

Unless explained otherwise, all technical and scientific terms used herein have the same meaning as commonly understood to one of ordinary skill in the art to which this disclosure belongs.

The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, second ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual, Cold Spring Harbor Laboratory Press, Cold Spring Harbor, N.Y. (1990), incorporated herein by reference. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

The term “coronavirus” or “CoV” refers to any virus of the coronavirus family, including but not limited to SARS-CoV-2, MERS-CoV, and SARS-CoV-1. SARS-CoV-2 refers to the newly emerged coronavirus which was identified as the cause of a serious outbreak starting in Wuhan, China, and which is rapidly spreading to other areas of the globe. SARS-CoV-2 has also been known as 2019-nCoV and Wuhan coronavirus. It binds via the viral spike protein to human host cell receptor angiotensin-converting enzyme 2 (ACE2). The spike protein also binds to and is cleaved by TMPRSS2, which activates the spike protein for membrane fusion of the virus.

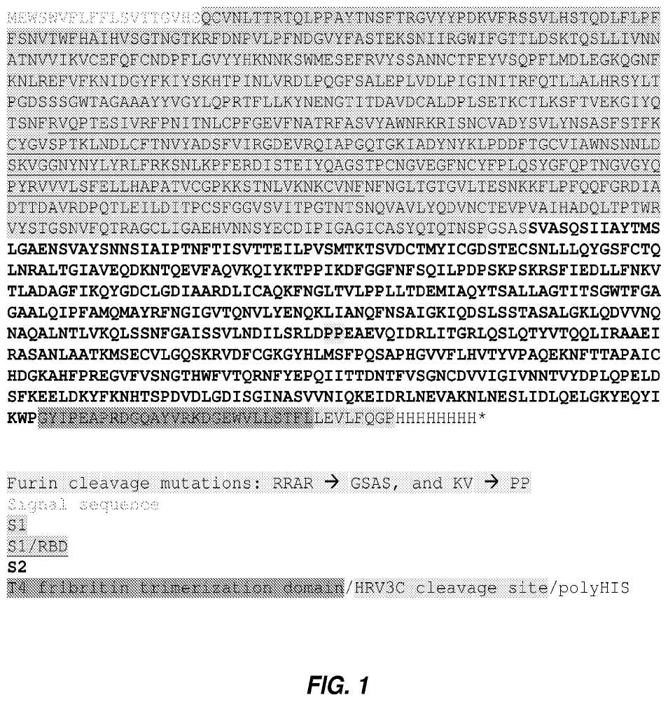

The term “CoV-S”, also called “S” or “S protein”, refers to the spike protein of SARS-CoV-2. The SARS-CoV-2-Spike protein is a 1273 amino acid type I membrane glycoprotein which assembles into trimers that constitute the spikes or peplomers on the surface of the enveloped coronavirus particle. The protein has two essential functions, host receptor binding and membrane fusion, which are attributed to the N-terminal (S1) and C-terminal (S2) halves of the S protein. CoV-S binds to its cognate receptor via a receptor binding domain (RBD) present in the S1 domain. The amino acid sequence of SARS-CoV-2 spike protein used in the present invention is exemplified by the amino acid sequence provided in SEQ ID NO: 1 (FIG. 1). The term “CoV-S” includes protein variants of SARS-CoV-2 spike protein isolated from different CoV isolates as well as recombinant CoV spike protein or a fragment thereof. The term also encompasses CoV spike protein or a fragment thereof coupled to, for example, a histidine tag, T4 fibritin trimerization domain, mouse or human Fc, or a signal sequence.

The term “coronavirus infection”, “SARS-CoV-2 infection”, or “CoV infection,” as used herein, refers to infection with a coronavirus such as SARS-CoV-2. The term includes coronavirus respiratory tract infections, often in the lower respiratory tract. Symptoms can include high fever, dry cough, shortness of breath, pneumonia, gastro-intestinal symptoms such as diarrhea, organ failure (kidney failure and renal dysfunction), septic shock, and death in severe cases.

The articles “a” and “an” are used herein to refer to one or to more than one (i.e. to at least one) of the grammatical object of the article.

Throughout this specification and claims, the word “comprise,” or variations such as “comprises” or “comprising,” will be understood to imply the inclusion of a stated integer or group of integers but not the exclusion of any other integer or group of integers.

The term “polypeptide” or “protein” encompasses native or artificial proteins, protein fragments and polypeptide analogs of a protein sequence. A polypeptide may be monomeric or polymeric.

“Peptide” refers to a polymer in which the monomers are amino acids and are joined together through amide bonds, alternatively referred to as a peptide. Additionally, unnatural amino acids, for example, β-alanine, phenylglycine and homoarginine are also included. Amino acids that are not nucleic acid-encoded may also be used in the present invention. Furthermore, amino acids that have been modified to include reactive groups, glycosylation sites, polymers, therapeutic moieties, biomolecules and the like may also be used in the invention. All of the amino acids used in the present invention may be either the D- or L-isomer thereof. The L-isomer is generally preferred. In addition, other peptidomimetics are also useful in the present invention. As used herein, “peptide” refers to both glycosylated and unglycosylated peptides. Also included are peptides that are incompletely glycosylated by a system that expresses the peptide. For a general review, see, Spatola, A. F., in CHEMISTRY AND BIOCHEMISTRY OF AMINO ACIDS, PEPTIDES AND PROTEINS, B. Weinstein, eds., Marcel Dekker, New York, p. 267 (1983).

The term “isolated protein”, “isolated polypeptide” or “isolated antibody” is a protein, polypeptide or antibody that by virtue of its origin or source of derivation (1) is not associated with naturally associated components that accompany it in its native state, (2) is free of other proteins from the same species, (3) is expressed by a cell from a different species, or (4) does not occur in nature. Thus, a polypeptide that is chemically synthesized or synthesized in a cellular system different from the cell from which it naturally originates will be “isolated” from its naturally associated components. A protein may also be rendered substantially free of naturally-associated components by isolation, using protein purification techniques well known in the art. The lower end of the range of purity for the isolated polypeptides is about 60%, about 70% or about 80% and the upper end of the range of purity is about 70%, about 80%, about 90% or more than about 90%.

When the polypeptides are more than about 90% pure, their purities are also preferably expressed as a range. The lower end of the range of purity is about 90%, about 92%, about 94%, about 96% or about 98%. The upper end of the range of purity is about 92%, about 94%, about 96%, about 98% or about 100% purity. An exemplary “isolated” polypeptide is a polypeptide that is at least about 95%, 98%, 99% or 99.5% pure.

Purity is determined by any art-recognized method of analysis (e.g., band intensity on a silver stained gel, polyacrylamide gel electrophoresis, HPLC, or a similar means).

The term “immunoglobulin (Ig)” as used herein refers to immunity conferring glycoproteins of the immunoglobulin superfamily. “Surface immunoglobulins” are attached to the membrane of effector cells by their transmembrane region and encompass molecules such as but not limited to B-cell receptors, T-cell receptors, class I and II major histocompatibility complex (MHC) proteins, beta-2 microglobulin (β2M), CD3, CD4 and CD8. Typically, the term “antibody” as used herein refers to secreted immunoglobulins which lack the transmembrane region and can thus, be released into the bloodstream and body cavities. Human antibodies are grouped into different isotypes based on the heavy chain they possess. There are five types of human Ig heavy chains denoted by the Greek letters: α, β, γ, and μ. The type of heavy chain present defines the class of antibody, i.e. these chains are found in IgA, IgD, IgE, IgG, and IgM antibodies, respectively, each performing different roles, and directing the appropriate immune response against different types of antigens. Distinct heavy chains differ in size and composition; α and γ and comprise approximately 450 amino acids, while μ has approximately 550 amino acids (Janeway et al. (2001) Immunobiology, Garland Science). IgA is found in mucosal areas, such as the gut, respiratory tract and urogenital tract, as well as in saliva, tears, and breast milk and prevents colonization by pathogens (Underdown & Schiff (1986) Annu. Rev. Immunol. 4:389-417). IgD mainly functions as an antigen receptor on B cells that have not been exposed to antigens and is involved in activating basophils and mast cells to produce antimicrobial factors (Geisberger et al. (2006) Immunology 118:429-437; Chen et al. (2009) Nat. Immunol. 10:889-898). IgE is involved in allergic reactions via its binding to allergens triggering the release of histamine from mast cells and basophils. IgE is also involved in protecting against parasitic worms (Pier et al. (2004) Immunology, Infection, and Immunity, ASM Press). IgG provides the majority of antibody-based immunity against invading pathogens and is the only antibody isotype capable of crossing the placenta to give passive immunity to fetus (Pier et al. (2004) Immunology, Infection, and Immunity, ASM Press). In humans there are four different IgG subclasses (IgG1, 2, 3, and 4), named in order of their abundance in serum with IgG1 being the most abundant (about 66%), followed by IgG2 (about 23%), IgG3 (about 7%) and IgG4 (about 4%). The biological profile of the different IgG classes is determined by the structure of the respective hinge region. IgM is expressed on the surface of B cells in a monomeric form and in a secreted pentameric form with very high avidity. IgM is involved in eliminating pathogens in the early stages of B cell mediated (humoral) immunity before sufficient IgG is produced (Geisberger et al. (2006) Immunology 118:429-437).

Antibodies are not only found as monomers but are also known to form dimers of two Ig units (e.g. IgA), tetramers of four Ig units (e.g. IgM of teleost fish), or pentamers of five Ig units (e.g. mammalian IgM). Antibodies are typically made of four polypeptide chains comprising two identical heavy chains and identical two light chains which are connected via disulfide bonds and resemble a “Y”-shaped macro-molecule. Each of the chains comprises a number of immunoglobulin domains out of which some are constant domains and others are variable domains. Immunoglobulin domains consist of a 2-layer sandwich of between 7 and 9 antiparallel β-strands arranged in two β-sheets. Typically, the “heavy chain” of an antibody comprises four Ig domains with three of them being constant (CH domains: CH1, CH2, CH3) domains and one of the being a variable domain (V), with the exception of IgM and IgE which contain one variable (VH) and four constant regions (CH1, CH2, CH3, CH4). The additional domain (CH2: Cμ2, C∈2) in the heavy chains of IgM and IgE molecules connects the two heavy chains instead of the hinge region contained in other Ig molecules (Perkins et al., (1991) J Mol Biol. 221(4):1345-66; Beavil et al., (1995) Biochem 34(44):14449-61; Wan et al., (2002) Nat Immunol. 3(7):681-6). The “light chain” typically comprises one constant Ig domain (CL) and one variable Ig domain (VL). Exemplified, the human IgM heavy chain is composed of four Ig domains linked from N- to C-terminus in the order VH-CH1-CH2-CH3-CH4 (also referred to as VH-Cμ1-Cμ2-Cμ3-Cμ4), whereas the human IgM light chain is composed of two immunoglobulin domains linked from N- to C-terminus in the order VL-CL, being either of the kappa or lambda type (Vκ-Cκ or Vλ-Cλ).

Exemplified, the constant chain of human IgM comprises 452 amino acids. Throughout the present specification and claims, the numbering of the amino acid positions in an immunoglobulin are that of the “EU index” as in Kabat, E. A., Wu, T. T., Perry, H. M., Gottesman, K. S., and Foeller, C., (1991) Sequences of proteins of immunological interest, 5th ed. U.S. Department of Health and Human Service, National Institutes of Health, Bethesda, Md. The “EU index as in Kabat” refers to the residue numbering of the human IgM EU antibody. Accordingly, CH domains in the context of IgM are as follows: “CH1” refers to amino acid positions 118-215 according to the EU index as in Kabat; “CH2” refers to amino acid positions 231-340 according to the EU index as in Kabat; “CH3” refers to amino acid positions 341-446 according to the EU index as in Kabat. “CH4” refers to amino acid positions 447-558 according to the OU index as in Kabat.

Whilst in human IgA, IgG, and IgD molecules two heavy chains are connected via their hinge region, IgE and IgM antibodies do not comprise such hinge region. Instead, IgE and IgM antibodies possess an additional Ig domain, their CH2 domain, which functions as dimerization domain between two heavy chains. In contrast to rather flexible and linear hinge regions of other antibodies, the CH2 domain of IgE and IgM are composed of two beta sheets stabilized by an intradomain disulfide bond forming a c-type immunoglobulin fold (Bork et al., (1994) J Mol Biol. 242(4):309-20; Wan et al., (2002) Nat Immunol. 3(7):681-6). Furthermore, the MHD2 and EHD2 domains contain one N-glycosylation site.

The “IgM heavy chain domain 2” (“MHD2”) consists of 111 amino acid residues (12.2 kDa) forming a homodimer covalently held together by a disulfide bond formed between cysteine residue 337 of two domains (Davis et al., (1989) EMBO J 8(9):2519-26; Davis & Shulman, (1989) Immunol Today. 10(4):118-22; 127-8). The domain is further stabilized by an intradomain disulfide bond formed between Cys261 and Cys321. Typically, two MHD2 domains are covalently linked by an interdomain disulfide bond between Cys337. The MHD2 contains an N-glycosylation site at Asn333.

“Fc” or “Fc region” or “Fc domain” as used herein refers to the polypeptide comprising the constant region of an antibody excluding the first constant region immunoglobulin domain and, in some cases, part of the hinge. Thus, Fc refers to the last two constant region immunoglobulin domains of IgA, IgD, and IgG, the last three constant region immunoglobulin domains of IgE and IgM, and the flexible hinge N-terminal to these domains. For IgA and IgM, Fc may or may not include the J chain. For IgG, the Fc domain comprises immunoglobulin domains Cγ2 and Cγ3 (Cγ2 and Cγ3) and the lower hinge region between Cγ1 (Cγ1) and Cγ2 (Cγ2). In some embodiments, as is more fully described below, amino acid modifications are made to the Fc region, for example to alter binding to one or more FcγR receptors or to the FcRn receptor.

As used herein, the term “human antibody” means any antibody in which the variable and constant domain sequences are human sequences. The term encompasses antibodies acquired from and/or enriched from a human sourced starting material, e.g., plasma from a recovered donor infected with SARS-CoV-2.

A “neutralizing antibody”, an antibody with “neutralizing activity”, “antagonistic antibody”, or “inhibitory antibody”, as used herein, means an antibody capable of preventing, retarding or diminishing replication of the viral target of the antibody. In some embodiments, neutralizing antibodies are effective at antibody concentrations of <0.2 μg/mL. In some embodiments, neutralizing antibodies are effective at antibody concentrations of <0.1 μg/mL. An exemplary neutralizing antibody “neutralizes” a virus (e.g., SARS-CoV-2) if it partly or fully impedes the virus' ability to infect a cell that, absent the antibody, it would otherwise infect, or if it prevents viral replication within an infected cell. An exemplary neutralizing antibody is one that neutralizes 200 times the tissue culture infectious dose required to infect 50% of cells (200×TCID₅₀) in the presence of the SARS-CoV-2. In some embodiments, neutralizing antibodies are effective at antibody concentrations of <12.5 μg/mL, <3.125 μg/mL, or <0.8 μg/mL. One measure for assessing the neutralization capacity of an antibody (or antigen-binding portion thereof) for inhibiting the ability of a pseudovirus or virus to infect cells involves a dose-response evaluation, which allows for the determination of the concentration of antibody (or antigen-binding portion thereof) required to neutralize 50% of infection (IC₅₀). IC₅₀ values can be calculated using the methods described in the accompanying Examples.

The term “TCID₅₀” refers to the amount of virus necessary to infect 50% of cells in tissue culture. 100× and 200× refer to 100 or 200 times the concentration of virus compared to the TCID₅₀. In a TCID₅₀ assay, serial dilutions of a virus are added onto monolayers of cells, and left until a cytopathic effect can be seen. From the resulting dose-response curve, it is possible to determine the accurate TC₅₀ values.

The term “K_{D}” refers to the equilibrium dissociation constant of a particular protein-ligand interaction. K_{D} values can be calculated using the methods described in the accompanying Examples.

The term “epitope” includes any protein determinant capable of specific binding to an immunoglobulin or T-cell receptor or otherwise interacting with a molecule. Epitopic determinants generally consist of chemically-active surface groupings of molecules such as amino acids or carbohydrate or sugar side chains and generally have specific three dimensional structural characteristics, as well as specific charge characteristics. An epitope may be “linear” or “conformational.” In a linear epitope, all of the points of interaction between the protein and the interacting molecule (such as an antibody) occur linearly along the primary amino acid sequence of the protein. In a conformational epitope, the points of interaction occur across amino acid residues on the protein that are separated from one another. An antibody is said to specifically bind an antigen when the dissociation constant is ≤1 mM, preferably ≤100 nM and most preferably ≤10 nM. In certain embodiments, the K_{D} is from about 1 pM to about 500 pM. In some embodiments, the K_{D} is from about 500 pM to about 1 μM. In some embodiments, the K_{D} is from about 1 μM to about 100 nM. In some embodiments, the K_{D} is from about 100 mM to about 10 nM. It is possible to competitively screen antibodies for binding to the same epitope. An approach to achieve this is to conduct cross-competition studies to find antibodies that competitively bind with one another, e.g., the antibodies compete for binding to the antigen. A high throughput process for “binding” antibodies based upon their cross-competition is described in International Patent Application No. WO 03/48731.

Methods for determining the epitope of an antigen-binding protein, e.g., antibody or fragment or polypeptide, include alanine scanning mutational analysis, peptide blot analysis (Reineke (2004) Methods Mol. Biol. 248: 443-63), peptide cleavage analysis, crystallographic studies and NMR analysis. In addition, methods such as epitope excision, epitope extraction and chemical modification of antigens can be employed (Tomer (2000) Prot. Sci. 9: 487-496). Another method that can be used to identify the amino acids within a polypeptide with which an antigen-binding protein (e.g., antibody or fragment or polypeptide) interacts is hydrogen/deuterium exchange detected by mass spectrometry. In general terms, the hydrogen/deuterium exchange method involves deuterium-labeling the protein of interest, followed by binding the antigen-binding protein, e.g., antibody or fragment or polypeptide, to the deuterium-labeled protein. Next, the CoV-S protein/antigen-binding protein complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antigen-binding protein interface may retain deuterium and therefore exhibit relatively higher mass compared to amino acids not included in the interface. After dissociation of the antigen-binding protein (e.g., antibody or fragment or polypeptide), the target protein is subjected to protease cleavage and mass spectrometry analysis, thereby revealing the deuterium-labeled residues which correspond to the specific amino acids with which the antigen-binding protein interacts. See, e.g., Ehring (1999) Analytical Biochemistry 267: 252-259; Engen and Smith (2001) Anal. Chem. 73: 256A-265A.

As used herein, certain binding molecules provided in this disclosure are “dimeric,” and include two bivalent binding units that include IgA constant regions or multimerizing fragments thereof. Certain binding molecules provided in this disclosure are “pentameric” or “hexameric,” and include five or six bivalent binding units that include IgM constant regions or multimerizing fragments thereof. A binding molecule, e.g., an antibody or antibody-like molecule, comprising two or more, e.g., two, five, or six binding units, is referred to herein as “multimeric.”

The term “fusion protein” or “fused protein”, as used interchangeably herein, refers to a protein coded by a single gene and the single gene is made up of coding sequences that originally coded for at least two or more separate proteins. A fusion protein may retain one or more functional domains of the two or more separate proteins. Part of the coding sequence for a fusion protein may code for an epitope tag. In certain embodiments, antibodies, or antigen binding portions thereof, may be present within a fusion protein.

A “disease” is a state of health of a subject wherein the subject cannot maintain homeostasis, and wherein if the disease is not ameliorated then the subject's health continues to deteriorate. An exemplary disease is infection by SARS-CoV-2 (COVID) or a symptom caused by such infection.

As used herein, “pharmaceutically acceptable carrier” includes solvents, dispersion media, coatings, antibacterial and antifungal agents, isotonic and absorption delaying agents, and the like that are physiologically compatible. Some examples of pharmaceutically acceptable carriers are water, saline, phosphate buffered saline, dextrose, glycerol, ethanol and the like, as well as combinations thereof. In many cases, it will be preferable to include isotonic agents, for example, sugars, polyalcohols such as mannitol, sorbitol, amino acids (e.g., glycine, proline, etc.), or sodium chloride in the composition. Additional examples of pharmaceutically acceptable substances are wetting agents or minor amounts of auxiliary substances such as wetting or emulsifying agents, preservatives or buffers, which enhance the shelf life or effectiveness of the antibody. Compositions comprising such carriers are formulated by well-known conventional methods. Exemplary formulations of the invention include one, two, or more, different amino acids. In an exemplary embodiment, the presence of the amino acid(s) improves the stability of the antibodies, even at high concentrations at which the antibody is typically not stable in formulations absinthe amino acid(s). In various embodiments, the carrier is selected to provide a “stable pharmaceutical formulation”.

The term “stable formulation” such as “stable pharmaceutical formulation” as used in connection with the formulations described herein denotes, without limitation, a formulation, which preserves its physical stability/identity/integrity and/or chemical stability/identity/integrity and/or biological activity/identity/integrity during manufacturing, storage and administration. Various analytical techniques for evaluating protein stability are available in the art and reviewed in Reubsaet, et al. (1998) J Pharm Biomed Anal 17(6-7): 955-78 and Wang, W. (1999) Int J Pharm 185(2): 129-88. Stability can be evaluated by, for example, without limitation, storage at selected climate conditions for a selected time period, by applying mechanical stress such as shaking at a selected shaking frequency for a selected time period, by irradiation with a selected light intensity for a selected period of time, or by repetitive freezing and thawing at selected temperatures. The stability may be determined by, for example, at least one of the methods selected from the group consisting of visual inspection, SDS-PAGE, IEF, size exclusion liquid chromatography (SEC-HPLC), reversed phase liquid chromatography (RP-HPLC), ion-exchange HPLC, capillary electrophoresis, light scattering, particle counting, turbidity, RFFIT, and kappa/lambda ELISA, without limitation. Exemplary characteristics of use with visual inspection include turbidity and aggregate formation.

In an embodiment, a formulation is considered stable when the protein in the formulation (1) retains its physical stability, (2) retains its chemical stability and/or (3) retains its biological activity.

A protein may be said to “retain its physical stability” in a formulation if, for example, without limitation, it shows no signs of aggregation, precipitation and/or denaturation upon visual examination of color and/or clarity, or as measured by UV light scattering or by size exclusion chromatography (SEC) or electrophoresis, such as with reference to turbidity or aggregate formation.

A protein may be said to “retain its chemical stability” in a formulation, if, for example, without limitation, the chemical stability at a given time is such that there is no significant modification of the protein by bond formation or cleavage resulting in a new chemical entity. In a further embodiment, chemical stability can be assessed by detecting and quantifying chemically altered forms of the protein. Chemical alteration may involve, example, without limitation, size modification (e.g. clipping) which can be evaluated using size exclusion chromatography, SDS-PAGE and/or matrix-assisted laser desorption ionization/time-of-flight mass spectrometry (MALDI/TOF MS). Other types of chemical alteration include, for example, without limitation, charge alteration (e.g. occurring as a result of deamidation), which can be evaluated by ion-exchange chromatography, for example. Oxidation is another commonly seen chemical modification.

In an embodiment, a protein may be said to “retain its biological activity” relative to native unmodified protein in a pharmaceutical formulation, if, for example, without limitation, the biological activity of the protein, at a given time is from about 50% to about 200%, or alternatively from about 60% to about 170%, or alternatively from about 70% to about 150%, or alternatively from about 80% to about 125%, or alternatively from about 90% to about 110%, of the biological activity exhibited at the time the formulation was prepared as determined, e.g., in an antigen binding assay or virus neutralization assay. In a further embodiment, a protein may be said to “retain its biological activity” in a pharmaceutical formulation, if, for example, without limitation, the biological activity of the protein, at a given time is at least 1%, 5%, 10%, 15%, 20%, 25%, 30%, 35%, 40%, 45%, 50%, 55%, 60%, 65%, 70%, 75%, 80%, 85%, 90%, 95% or 100%.

In one embodiment, a stable pharmaceutical formulation contains one or more proteins and at least one amino acid selected based on the amino acid's ability to increase the stability of the protein and/or reduce solution viscosity. In one embodiment, the amino acid contains a positively charged side chain, such as R, H, and K. In some embodiments, the amino acid contains a negatively charged side chain, such as D and E. In some embodiments, the amino acid contains a hydrophobic side chain, such as A, F, I, L, M, V, W, and Y. In some embodiments, the amino acid contains a polar uncharged side chain, such as S, T, N, and Q. In some embodiments, the amino acid does not have a side chain, i.e., G.

In one embodiment, the amino acid is any one of A, N, D, Q, E, I, L, K, F, P, S, T, W, Y, or V.

As used herein, the term “amino acid” refers to either natural and/or unnatural or synthetic amino acids.

The term “in vivo” refers to an event occurring in a subject's body.

The term “in vitro” refers to an event that occurring outside of a subject's body. In vitro assays encompass cell-based assays in which cells alive or dead are employed and may also encompass a cell-free assay in which no intact cells are employed.

“Linker”, or grammatical equivalents thereof, as used herein, means a linker joining two or more amino acids, or two or more peptides together. As is more fully described below, generally, there are a number of suitable linkers that can be used, including traditional peptides, produced by chemical synthetic methods or generated by recombinant techniques.

“Modified” or “modification”, as used herein, means an amino acid substitution, insertion, and/or deletion in a polypeptide sequence or an alteration to a moiety chemically linked to a polypeptide. For example, a modification may be an altered carbohydrate or PEG structure attached to a polypeptide. For clarity, unless otherwise noted, the amino acid modification is always applied to an amino acid coded by DNA, e.g., the 20 amino acids that have codons in DNA and RNA.

“Conservative substitutions” will produce molecules having functional and chemical characteristics similar to those of the molecule from which such modifications are made. For example, a “conservative amino acid substitution” may involve a substitution of an amino acid residue with another residue such that there is little or no effect on the polarity or charge of the amino acid residue at that position. Desired amino acid substitutions (whether conservative or non-conservative) can be determined by those skilled in the art. For example, amino acid substitutions can be used to identify important residues of the molecule sequence, or to increase or decrease the affinity of the molecules described herein. Variants comprising one or more conservative amino acid substitutions can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

“Amino acid insertion” or “insertion”, as used herein, means the addition of an amino acid sequence at a particular position in a parent polypeptide sequence.

“Amino acid deletion” or “deletion”, as used herein, means the removal of an amino acid sequence at a particular position in a parent polypeptide sequence.

“Fused”, as used herein, means the components (e.g., a polypeptide and a tag) are linked by covalent bonds, either directly or indirectly via linkers.

The polypeptides of the present invention are generally recombinant. “Recombinant” means the polypeptides are generated using recombinant nucleic acid techniques in exogenous host cells.

“Specific binding” or “specifically binds to”, as used herein, means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

As used herein, the term “expression” refers to transcription of a polynucleotide from a DNA template, resulting in, for example, an mRNA or other RNA transcript (e.g., non-coding, such as structural or scaffolding RNAs). The term further refers to the process through which transcribed mRNA is translated into peptides, polypeptides, or proteins. Transcripts and encoded polypeptides may be referred to collectively as “gene product.” Expression may include splicing the mRNA in a eukaryotic cell, if the polynucleotide is derived from genomic DNA.

In some embodiments, reduced expression of the target polynucleotide sequence is observed. The terms “decrease,” “reduced,” “reduction,” and “decrease” are all used herein generally to mean a decrease by a statistically significant amount. However, for avoidance of doubt, “decrease,” “reduced,” “reduction,” “decrease” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease from about 10-100% as compared to a reference level.

The terms “increased”, “increase” or “enhance” or “activate” are all used herein to generally mean an increase by a statically significant amount; for the avoidance of any doubt, the term “increased”, “increase” or “enhance” or “activate” means an increase of at least 10% as compared to a reference level, for example an increase of at least about 10%, or at least about 20%, or at least about 30%, or at least about 40%, or at least about 50%, or at least about 60%, or at least about 70%, or at least about 80%, or at least about 90% or up to and including a 100% increase or any increase from about 10-100% as compared to a reference level, or at least about a 2-fold, or at least about a 3-fold, or at least about a 4-fold, or at least about a 5-fold or at least about a 10-fold increase, or any increase from about 2-fold to about 10-fold or greater as compared to a reference level.

The terms “inactivate” and “inactivation” are used herein to generally mean that the expression of a gene of interest is reduced as compared to a reference level or not expressed in a functional or active protein form. The terms “partially inactivate” and “partial inactivation” refer to an expression of the gene of interest that is reduced but not eliminated as compared to a reference level, or that a percentage of the proteins expressed by the gene still retain their activity and function. The terms “fully inactivate” and “full inactivation” as used herein mean that the gene of interest does not express any protein, or all of the expressed proteins encoded by the gene of interest are inactive and nonfunctional.

The terms “inhibitors,” “activators,” and “modulators” as used herein refer to agents that affect a function or expression of a biologically-relevant molecule. The term “modulator” includes both inhibitors and activators. They may be identified using in vitro and in vivo assays for expression or activity of a target molecule. In some cases, “inhibitors” are agents that, e.g., inhibit expression or bind to target molecules or proteins. They may partially or totally block stimulation or have protease inhibitor activity. They may reduce, decrease, prevent, or delay activation, including inactivation, desensitization, or down regulation of the activity of the described target protein. Modulators may be antagonists or agonists of the target molecule or protein. In some cases, “activators” are agents that, e.g., induce or activate the function or expression of a target molecule or protein. They may bind to, stimulate, increase, open, activate, or facilitate the target molecule activity. Activators may be agonists of the target molecule or protein.

The terms “subject”, “host”, and “individual” are used interchangeably herein, and refer to an animal, for example, a human from whom cells can be obtained and/or to whom treatment, including prophylactic treatment, with the cells as described herein, is provided. For treatment of those infections, conditions or disease states which are specific for a specific animal such as a human subject, the term subject refers to that specific animal. The terms “non-human animals” and “non-human mammals” as used interchangeably herein, include mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g. dog, cat, horse, and the like, or production mammal, e.g. cow, sheep, pig, and the like.

“Percent (%) amino acid sequence identity” or “amino acid sequence with percent (%) identity” with respect to a protein sequence is defined as the percentage of amino acid residues in a candidate sequence that are identical with the amino acid residues in the specific (parental) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. The degree of identity between an amino acid sequence of the present invention (“invention sequence”) and the parental amino acid sequence is calculated as the number of exact matches in an alignment of the two sequences, divided by the length of the “invention sequence”, or the length of the parental sequence, whichever is the shortest. The result is expressed in percent identity.

The term “vaccination” or “vaccinate” means administration of a vaccine that can elicit an immune response or confer immunity from a disease.

A “protein tag” or “tag” refers to an amino acid sequence within a recombinant protein that provides new characteristics to the recombinant protein that assist in protein purification, identification, or activity based on the tag's characteristics and affinity. A protein tag may provide a novel enzymatic property to the recombinant protein such as a biotin tag, or a tag may provide a means of protein identification such as with fluorescence tags encoding for green fluorescent protein or red fluorescent protein. Protein tags may be added onto the N- or C-terminus of a protein. A common protein tag used in protein purification is a poly-His tag where a series of approximately six histidine amino acid residues are added which enables the protein to bind to protein purification matrices chelated to metal ions such as nickel or cobalt. Other tags commonly used in protein purification include chitin binding protein, maltose binding protein, glutathione-S-transferase, Myc tag, and FLAG-tag. Tags such as “epitope tags” may also confer the protein to have an affinity towards an antibody. Common antibody epitope tags include the V5-tag, Myc-tag, and HA-tag.

A “J-chain” as used herein refers to an acidic 15-kDa polypeptide, which is associated with pentameric IgM and dimeric IgA via disulfide bonds involving the penultimate cysteine residue in the 18-amino acid secretory tail-piece (tp) at the C-terminus of the IgM p or IgA a heavy chain. The three disulfide bridges are formed between Cys 12 and 100, Cys 71 and 91, and Cys 108 and 133, respectively. See, e.g. Frutiger et al. 1992, Biochemistry 31, 12643-12647. Structural requirements for incorporation of the J-chain into human IgM and IgA and for polymeric immunoglobulin assembly and association with the J-chain are reported by Sorensen et al. 2000, Int. Immunol. 12(1): 19-27 and Yoo et al. 1999, J Biol. Chem. 274(47):33771-33777, respectively. Recombinant production of soluble J-chain in E. coli is reported by Redwan et al. 2006, Human Antibodies 15:95-102.

The term “adjuvant” refers to agents that augment, stimulate, activate, potentiate, or modulate the immune response to the active ingredient of the composition at either the cellular or humoral level, e.g. immunologic adjuvants stimulate the response of the immune system to the actual antigen, but have no immunological effect themselves. Adjuvants are used to accomplish three objectives: (1) they slow the release of antigens from the injection site; (2) they stimulate the immune system; and (3) the addition of an adjuvant may permit the use of a smaller dose of antigen to stimulate a similar immune response, thereby reducing the production cost of the vaccine. Examples of such adjuvants include but are not limited to inorganic adjuvants (e.g. inorganic metal salts such as aluminium phosphate or aluminium hydroxide), organic adjuvants (e.g. saponins or squalene), oil-based adjuvants (e.g. Freund's complete adjuvant and Freund's incomplete adjuvant), cytokines (e.g. IL-1β, IL-2, IL-7, IL-12, IL-18, GM-CFS, and INF-γ) particulate adjuvants (e.g. immuno-stimulatory complexes (ISCOMS), liposomes, or biodegradable microspheres), virosomes, bacterial adjuvants (e.g. monophosphoryl lipid A, or muramyl peptides), synthetic adjuvants (e.g. non-ionic block copolymers, muramyl peptide analogues, or synthetic lipid A), or synthetic polynucleotides adjuvants (e.g. polyarginine or polylysine).

“Cytotoxic T lymphocyte” (CTL) as used herein refers to a T lymphocyte that expresses CD8 on the surface thereof (i.e., a CD8+ T cell). In some embodiments such cells are preferably “memory” T cells (TM cells) that are antigen-experienced.

“Central memory” T cell (or “TCM”) as used herein refers to an antigen experienced CTL that expresses CD62L or CCR7 and CD45RO on the surface thereof, and does not express or has decreased expression of CD45RA as compared to naive cells. In embodiments, central memory cells are positive for expression of CD62L, CCR7, CD28, CD127, CD45RO, and CD95, and have decreased expression of CD54RA as compared to naive cells.

“Effector memory” T cell (or “TEM”) as used herein refers to an antigen experienced T cell that does not express or has decreased expression of CD62L on the surface thereof as compared to central memory cells, and does not express or has decreased expression of CD45RA as compared to naive cell. In some embodiments, effector memory cells are negative for expression of CD62L and CCR7, compared to naive cells or central memory cells, and have variable expression of CD28 and CD45RA.

“Naive” T cells as used herein refers to a non antigen experienced T lymphocyte that expresses CD62L and CD45RA, and does not express CD45RO− as compared to central or effector memory cells. In some embodiments, naive CD8+ T lymphocytes are characterized by the expression of phenotypic markers of naive T cells including CD62L, CCR7, CD28, CD127, and CD45RA.

“Effector” “TE” T cells as used herein refers to a antigen experienced cytotoxic T lymphocyte cells that do not express or have decreased expression of CD62L, CCR7, CD28, and are positive for granzyme B and perforin as compared to central memory or naive T cells.

As used herein, “administering” or “administered” means, intravenous, intranasal, intraperitoneal, intramuscular, intralesional, or subcutaneous administration, intrathecal administration, or instillation into a surgically created pouch or surgically placed catheter or device to the subject.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population of subjects. Alternatively, when prevention is not possible, therapeutic intervention for inhibiting progression of the disease state (COVID) is contemplated (see “treating” infra).

The terms “condition,” “disease,” and “disorder” are used interchangeably.

The term “unit dosage form” refers to physically discrete units suitable as unitary dosages for human subjects and other mammals, each unit containing a predetermined quantity of active material calculated to produce the desired therapeutic effect, in association with a suitable pharmaceutical excipient. The unit dosage forms may be administered once or multiple unit dosages may be administered, for example, throughout an organ, or solid tumor.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactically effective amount. In certain embodiments, an effective amount is the amount of a compound or pharmaceutical composition described herein in a single dose. In certain embodiments, an effective amount is the combined amounts of a compound or pharmaceutical composition described herein in multiple doses.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

A “prophylactically effective amount” of a compound described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

As used herein, “reducing the likelihood” of a human subject's becoming symptomatic of a SARS-CoV-2 infection includes, without limitation, reducing such likelihood by at least 10%, at least 20%, at least 30%, at least 40%, at least 50%, at least 60%, at least 70%, at least 80%, at least 90%, or at least 95%. In various embodiments, these percentages are relevant to the likelihood of infection in a similar subject having had or likely to have similar exposure as the subject to whom the prophylactically effective amount of a pharmaceutical formulation of the invention is administered. Preferably, reducing the likelihood of a human subject's becoming symptomatic of a SARS-CoV-2 infection means preventing the subject from becoming symptomatic of a SARS-CoV-2 infection.

In an exemplary embodiment, the subject administered a prophylactically effective amount of the pharmaceutical formulation of the invention is at risk of being exposed to SARS-CoV-2. As used herein, an event wherein a subject is “at risk of being exposed” to SARS-CoV-2 includes, without limitation, an event wherein the subject may come into close contact with aerosols derived from tissue or secretions (e.g., the mucous membrane secretions) of infected animals, including infected human subjects.

In an exemplary embodiment, the subject has or may have recently been exposed to SARS-CoV-2. As used herein, a subject who “has or may have recently been exposed to” SARS-CoV-2 includes, for example, a subject who experienced a high risk event (e.g., one in which he/she may have come into close contact with tissue or aerosols derived from the tissue of infected animals, including infected human subjects) within the past month, three weeks, two weeks, one week, five days, four days, three days, two days or 24 hours.

As used herein, a human subject is “symptomatic” of a SARS-CoV-2 infection if the subject shows one or more symptoms known to appear in a SARS-CoV-2-infected human subject after a suitable incubation period. Such symptoms include, without limitation, detectable SARS-CoV-2 in the subject, and those symptoms shown by patients afflicted with SARS-CoV-2. SARS-CoV-2-related symptoms include, without limitation, respiratory distress, hypoxia, difficulty breathing (dyspnea), cardiovascular collapse, arrhythmia (e.g., atrial fibrillation, tachycardia, bradycardia), fatigue, altered mental status (including confusion), cough, fever, chills, abnormal blood coagulation events, myalgia, loss of smell and/or taste, loss of appetite, nausea, red/watery eyes, dizziness, stomach-ache, rash, sneezing, sputum/phlegm, and runny nose.

As used herein, “treating” a subject infected with SARS-CoV-2 and symptomatic of that infection includes, without limitation, (i) slowing, stopping or reversing the progression of one or more of the symptoms, (ii) slowing, stopping or reversing the progression of illness underlying such symptoms, (iii) reducing or eliminating the likelihood of the symptom's recurrence, and/or (iv) slowing the progression of, lowering or eliminating the infection. In one exemplary embodiment, treating a subject infected with SARS-CoV-2 and symptomatic of that infection includes (i) reversing the progression of one or more of the symptoms, (ii) reversing the progression of illness underlying such symptoms, (iii) preventing the recurrence of a symptom or symptoms, and/or (iv) eliminating the infection. The progress of treating a subject infected with SARS-CoV-2 and symptomatic of that infection can be measured according to a number of clinical endpoints. These include, without limitation, lower or negative viral titer (also known as viral load) and the amelioration or elimination of one or more SARS-CoV-2 symptoms. In various embodiments, the invention provides for treatment of subject who are infected with SARS-CoV-2 and have no limiting symptoms from this infection.

In an exemplary embodiment, treating reduces the risk of mortality of the subject. In some embodiments, treatment results in shortened time of recovery. In one embodiment, the progress of treating a subject infected with SARS-CoV-2 and symptomatic of that infection can be measured by using RNA PCR to test for lower or negative viral titer in total lung tissue and/or sputum.

In exemplary embodiments, treatment results in one or more desirable clinical results including reduction of risk of mortality, and/or shortened time to recovery from an active SARS-CoV-2 infection.

In various embodiments, “treating” a subject infected with SARS-CoV-2 with a pharmaceutical formulation of the invention results in one or more improvements of the clinical status of the patient with respect to: fever or feeling feverish/chills; cough; sore throat; runny or stuffy nose; sneezing; muscle or body aches; headaches; fatigue (tiredness); vomiting; diarrhea; respiratory tract infection; chest discomfort; shortness of breath; bronchitis; and/or pneumonia, which sign or symptom is secondary to viral infection. In addition, “treating” may result in regression or elimination or inhibiting the need for supplemental oxygen, the need for mechanical breathing assistance, or any other COVID-19 symptom that requires the patient to be hospitalized. Symptoms that may require hospitalization include a number of more severe SARS-CoV-2-related symptoms defined above.

The term “nucleic acid” includes RNA or DNA molecules having more than one nucleotide in any form including single-stranded, double-stranded, oligonucleotide or polynucleotide.

The terms “vector” and “plasmid” are used interchangeably and as used herein refer to a polynucleotide vehicle to introduce genetic material into a cell. Vectors can be linear or circular. Vectors can integrate into a target genome of a host cell or replicate independently in a host cell. Vectors can comprise, for example, an origin of replication, a multicloning site, and/or a selectable marker. An expression vector typically comprises an expression cassette. Vectors and plasmids include, but are not limited to, integrating vectors, prokaryotic plasmids, eukaryotic plasmids, plant synthetic chromosomes, episomes, viral vectors, cosmids, and artificial chromosomes. The term “vector” also includes both viral and nonviral means for introducing a nucleic acid molecule into a cell in vitro, in vivo, or ex vivo. Vectors may be introduced into the desired host cells by well-known methods, including, but not limited to, transfection, transduction, cell fusion, and lipofection. Vectors can comprise various regulatory elements including promoters.

Reference will now be made in detail to implementation of exemplary embodiments of the present disclosure. Those of ordinary skill in the art will understand that the following detailed description is illustrative only and it is not intended to be in any way limiting. The embodiments of the present disclosure will readily suggest themselves to such skilled persons having benefit of this disclosure.

In the interest of clarity, not all of the routine features of the implementations described herein are shown and described. It will be appreciated that, in the development of any such actual implementation, numerous implementation-specific decisions are made in order to achieve the developer's specific goals, such as compliance with application- and business-related constraints, and that these specific goals will vary from one implementation to another and from one developer to another. Moreover, it will be appreciated that such a development effort might be complex and time-consuming but would nevertheless be a routine undertaking of engineering for those of ordinary skill in the art having the benefit of this disclosure.

Many modifications and variations of the exemplary embodiments set forth in this disclosure are made without departing from the spirit and scope of the exemplary embodiments, as will be apparent to those skilled in the art. The specific exemplary embodiments described herein are offered by way of example only, and the disclosure is to be limited only by the terms of the appended claims, along with the full scope of equivalents to which such claims are entitled.

### B. Virus

The present invention includes methods for treating or preventing a viral infection in a subject. The term “virus” includes any virus whose infection in the body of a subject is treatable or preventable by administration of an anti-CoV-S antibody or antigen-binding fragment thereof (e.g., wherein infectivity of the virus is at least partially dependent on CoV-S). In an embodiment of the invention, a “virus” is any virus that expresses spike protein (e.g., CoV-S). The term “virus” also includes a CoV-S-dependent respiratory virus which is a virus that infects the respiratory tissue of a subject (e.g., upper and/or lower respiratory tract, trachea, bronchi, lungs) and is treatable or preventable by administration of an anti-CoV-S antibody or antigen-binding fragment thereof. For example, in an embodiment of the invention, virus includes coronavirus, SARS-CoV-2 (severe acute respiratory syndrome coronavirus 2), SARS-CoV-1 (severe acute respiratory syndrome coronavirus 1), and MERS-CoV (Middle East respiratory syndrome (MERS) coronavirus). Coronaviruses can include the genera of alphacoronaviruses, betacoronaviruses, gammacoronaviruses, and deltacoronaviruses. In some embodiments, the antibodies or antigen-binding fragments provided herein can bind to and/or neutralize an alphacoronavirus, a betacoronavirus, a gammacoronavirus, and/or a deltacoronavirus. In certain embodiments, this binding and/or neutralization can be specific for a particular genus of coronavirus or for a particular subgroup of a genus. “Viral infection” refers to the invasion and multiplication of a virus in the body of a subject.

Coronavirus virions are spherical with diameters of approximately 125 nm. The most prominent feature of coronaviruses is the club-shape spike projections emanating from the surface of the virion. These spikes are a defining feature of the virion and give them the appearance of a solar corona, prompting the name, coronaviruses. Within the envelope of the virion is the nucleocapsid. Coronaviruses have helically symmetrical nucleocapsids, which is uncommon among positive-sense RNA viruses, but far more common for negative-sense RNA viruses. SARS-CoV-2, MERS-CoV, and SARS-CoV-1 belong to the coronavirus family. The initial attachment of the virion to the host cell is initiated by interactions between the S protein and its receptor. The sites of receptor binding domains (RBD) within the S1 domain of a coronavirus S protein vary depending on the virus, with some having the RBD at the C-terminus of S1. The S-protein/receptor interaction is the primary determinant for a coronavirus to infect a host species and also governs the tissue tropism of the virus. Many coronaviruses utilize peptidases as their cellular receptor. Following receptor binding, the virus must next gain access to the host cell cytosol. This is generally accomplished by acid-dependent proteolytic cleavage of S protein by a cathepsin, TMPRRS2 or another protease, followed by fusion of the viral and cellular membranes.

### C. Antibody

Accordingly, the invention provides a pharmaceutical composition comprising an anti-CoV-S antibody. The antibodies of the invention are specific for the spike protein of SARS-CoV-2 as more fully outlined herein and below.

As is discussed below, the term “antibody” is used generally. Antibodies that find use in the present invention can take on a number of formats as described herein, including traditional antibodies as well as antibody derivatives, fragments and mimetics, described below. In general, the term “antibody” includes any polypeptide that includes at least one antigen binding domain, as more fully described below. Antibodies may be polyclonal, monoclonal, xenogeneic, allogeneic, syngeneic, or modified forms thereof, as described herein, with monoclonal antibodies finding particular use in many embodiments. In some embodiments, antibodies of the invention bind specifically or substantially specifically to CoV-S. The terms “monoclonal antibodies” and “monoclonal antibody composition”, as used herein, refer to a population of antibody molecules that contain only one species of an antigen-binding site capable of immunoreacting with a particular epitope of an antigen, whereas the term “polyclonal antibodies” and “polyclonal antibody composition” refer to a population of antibody molecules that contain multiple species of antigen-binding sites capable of interacting with a particular antigen. A monoclonal antibody composition, typically displays a single binding affinity for a particular antigen with which it immunoreacts.

Traditional full-length antibody structural units typically comprise a tetramer. Each tetramer is typically composed of two identical pairs of polypeptide chains, each pair having one “light” (typically having a molecular weight of about 25 kDa) and one “heavy” chain (typically having a molecular weight of about 50-70 kDa). Human light chains are classified as kappa and lambda light chains. The present invention is directed to the IgG class, which has several subclasses, including, but not limited to IgG1, IgG2, IgG3, and IgG4. Thus, “isotype” as used herein is meant any of the subclasses of immunoglobulins defined by the chemical and antigenic characteristics of their constant regions. While the exemplary antibodies herein are based on IgG2 heavy constant regions, the anti-CoV-S antibodies of the invention include those using IgG1, IgG3 and IgG4 sequences, or combinations thereof. For example, as is known in the art, different IgG isotypes have different effector functions which may or may not be desirable. Accordingly, the antibodies of the invention can also swap out the IgG2 constant domains for IgG1, IgG3 or IgG4 constant domains.

The amino-terminal portion of each chain includes a variable region of about 100 to 110 or more amino acids primarily responsible for antigen recognition, generally referred to in the art and herein as the “Fv domain” or “Fv region”. In the variable region, three loops are gathered for each of the V domains of the heavy chain and light chain to form an antigen-binding site. Each of the loops is referred to as a complementarity-determining region (hereinafter referred to as a “CDR”), in which the variation in the amino acid sequence is most significant. “Variable” refers to the fact that certain segments of the variable region differ extensively in sequence among antibodies. Variability within the variable region is not evenly distributed. Instead, the V regions consist of relatively invariant stretches called framework regions (FRs) of 15-30 amino acids separated by shorter regions of extreme variability called “hypervariable regions”.

Each VH and VL is composed of three hypervariable regions (“complementary determining regions,” “CDRs”) and four FRs, arranged from amino-terminus to carboxy-terminus in the following order: FR1-CDR1-FR2-CDR2-FR3-CDR3-FR4.

The hypervariable region generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region, although sometimes the numbering is shifted slightly as will be appreciated by those in the art; Kabat et al., SEQUENCES OF PROTEINS OF IMMUNOLOGICAL INTEREST, 5 th Ed. Public Health Service, National Institutes of Health, Bethesda, Md. (1991) and/or those residues forming a hypervariable loop (e.g. residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917.

The carboxy-terminal portion of each chain defines a constant region primarily responsible for effector function. Kabat et al. collected numerous primary sequences of the variable regions of heavy chains and light chains. Based on the degree of conservation of the sequences, they classified individual primary sequences into the CDR and the framework and made a list thereof (see SEQUENCES OF IMMUNOLOGICAL INTEREST, 5 th edition, NIH publication, No. 91-3242, E. A. Kabat et al., entirely incorporated by reference).

In the IgG subclass of immunoglobulins, there are several immunoglobulin domains in the heavy chain. By “immunoglobulin (Ig) domain” herein is meant a region of an immunoglobulin having a distinct tertiary structure. Of interest in the present invention are the heavy chain domains, including, the constant heavy (CH) domains and the hinge domains. In the context of IgG antibodies, the IgG isotypes each have three CH regions. Accordingly, “CH” domains in the context of IgG are as follows: “CH1” refers to positions 118-220 according to the EU index as in Kabat. “CH2” refers to positions 237-340 according to the EU index as in Kabat, and “CH3” refers to positions 341-447 according to the EU index as in Kabat.

Accordingly, the invention provides variable heavy domains, variable light domains, heavy constant domains, light constant domains and Fc domains to be used as outlined herein. By “variable region” as used herein is meant the region of an immunoglobulin that comprises one or more Ig domains substantially encoded by any of the Vκ or Vλ, and/or VH genes that make up the kappa, lambda, and heavy chain immunoglobulin genetic loci respectively. Accordingly, the variable heavy domain comprises vhFR1-vhCDR1-vhFR2-vhCDR2-vhFR3-vhCDR3-vhFR4, and the variable light domain comprises vlFR1-vlCDR1-vlFR2-vlCDR2-vlFR3-vlCDR3-vlFR4. By “heavy constant region” herein is meant the CH1-hinge-CH2-CH3 portion of an antibody. By “Fc” or “Fc region” or “Fc domain” as used herein is meant the polypeptide comprising the constant region of an antibody excluding the first constant region immunoglobulin domain and in some cases, part of the hinge. Thus, Fc refers to the last two constant region immunoglobulin domains of IgA, IgD, and IgG, the last three constant region immunoglobulin domains of IgE and IgM, and the flexible hinge N-terminal to these domains. For IgA and IgM, Fc may include the J chain. For IgG, the Fc domain comprises immunoglobulin domains Cγ2 and Cγ3 (Cγ2 and Cγ3) and the lower hinge region between Cγ1 (Cγ1) and Cγ2 (Cγ2). Although the boundaries of the Fc region may vary, the human IgG heavy chain Fc region is usually defined to include residues C226 or P230 to its carboxyl-terminus, wherein the numbering is according to the EU index as in Kabat. In some embodiments, as is more fully described below, amino acid modifications are made to the Fc region, for example to alter binding to one or more FcγR receptors or to the FcRn receptor.

Thus, “Fc variant” or “variant Fc” as used herein is meant a protein comprising an amino acid modification in an Fc domain. The Fc variants of the present invention are defined according to the amino acid modifications that compose them. Thus, for example, N434S or 434S is an Fc variant with the substitution serine at position 434 relative to the parent Fc polypeptide, wherein the numbering is according to the EU index. Likewise, M428L/N434S defines an Fc variant with the substitutions M428L and N434S relative to the parent Fc polypeptide. The identity of the WT amino acid may be unspecified, in which case the aforementioned variant is referred to as 428L/434S. It is noted that the order in which substitutions are provided is arbitrary, that is to say that, for example, 428L/434S is the same Fc variant as M428L/N434S, and so on. For all positions discussed in the present invention that relate to antibodies, unless otherwise noted, amino acid position numbering is according to the EU index.

By “Fab” or “Fab region” as used herein is meant the polypeptide that comprises the VH, CH1, VL, and CL immunoglobulin domains. Fab may refer to this region in isolation, or this region in the context of a full length antibody, antibody fragment or Fab fusion protein. By “Fv” or “Fv fragment” or “Fv region” as used herein is meant a polypeptide that comprises the VL and VH domains of a single antibody. As will be appreciated by those in the art, these generally are made up of two chains.

Throughout the present specification, either the IMTG numbering system or the Kabat numbering system is generally used when referring to a residue in the variable domain (approximately, residues 1-107 of the light chain variable region and residues 1-113 of the heavy chain variable region) (e.g., Kabat et al., supra (1991)). EU numbering as in Kabat is generally used for constant domains and/or the Fc domains.

The CDRs contribute to the formation of the antigen-binding, or more specifically, epitope binding site of antibodies. “Epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope.

The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and nonconformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning”. Specific bins are described below.

Included within the definition of “antibody” is an “antigen-binding portion” of an antibody (also used interchangeably with “antigen-binding fragment”, “antibody fragment” and “antibody derivative”). That is, for the purposes of the invention, an antibody of the invention has a minimum functional requirement that it bind to CoV-S antigen. As will be appreciated by those in the art, there are a large number of antigen fragments and derivatives that retain the ability to bind an antigen and yet have alternative structures, including, but not limited to, (i) the Fab fragment consisting of VL, VH, CL and CH1 domains, (ii) the Fd fragment consisting of the VH and CH1 domains, (iii) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al., 1988, Science 242:423-426, Huston et al., 1988, Proc. Natl. Acad. Sci. U.S.A. 85:5879-5883, entirely incorporated by reference), (iv) “diabodies” or “triabodies”, multivalent or multispecific fragments constructed by gene fusion (Tomlinson et. al., 2000, Methods Enzymol. 326:461-479; WO94/13804; Holliger et al., 1993, Proc. Natl. Acad. Sci. U.S.A. 90:6444-6448, all entirely incorporated by reference), (v) “domain antibodies” or “dAb” (sometimes referred to as an “immunoglobulin single variable domain”, including single antibody variable domains from other species such as rodent (for example, as disclosed in WO 00/29004), nurse shark and Camelid V-HH dAbs, (vi) SMIPs (small molecule immunopharmaceuticals), camelbodies, nanobodies and IgNAR.

Still further, an antibody or antigen-binding portion thereof (antigen-binding fragment, antibody fragment, antibody portion) may be part of a larger immunoadhesion molecules (sometimes also referred to as “fusion proteins”), formed by covalent or noncovalent association of the antibody or antibody portion with one or more other proteins or peptides. Examples of immunoadhesion molecules include use of the streptavidin core region to make a tetrameric scFv molecule and use of a cysteine residue, a marker peptide and a C-terminal polyhistidine tag to make bivalent and biotinylated scFv molecules. Antibody portions, such as Fab and F(ab′)2 fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein.

In general, the anti-CoV-S antibodies of the invention are recombinant. “Recombinant” as used herein, refers broadly with reference to a product, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “recombinant antibody”, as used herein, includes all antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. Alternatively, such recombinant human antibodies have variable regions in which the framework are derived from human germline immunoglobulin sequences and CDR sequences can be any of those described herein (see FIGS. 12A-12PP). In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In some antibodies only part of a CDR, namely the subset of CDR residues required for binding termed the “specificity determining residues” (“SDRs”), are needed to retain binding of the antibody. CDR residues not contacting antigen and not in the SDRs can be identified based on previous studies from regions of Kabat CDRs lying outside Chothia hypervariable loops (see Kabat et al., SEQUENCES OF PROTEINS OF IMMUNOLOGICAL INTEREST, National Institutes of Health Publication No. 91-3242 (1992); Chothia et al., “Canonical Structures For The Hypervariable Regions of Immunoglobulins,” J. Mol. Biol. 196:901-917 (1987), which are hereby incorporated by reference in their entirety), by molecular modeling and/or empirically, or as described in Gonzales et al., “SDR Grafting of a Murine Antibody Using Multiple Human Germline Templates to Minimize Its Immunogenicity,” Mol. Immunol. 41:863-872 (2004), which is hereby incorporated by reference in its entirety. In such humanized antibodies, at positions in which one or more donor CDR residues is absent or in which an entire donor CDR is omitted, the amino acid occupying the position can be an amino acid occupying the corresponding position (by Kabat numbering) in the acceptor antibody sequence. The number of such substitutions of acceptor for donor amino acids in the CDRs to include reflects a balance of competing considerations. Such substitutions are potentially advantageous in decreasing the number of mouse amino acids in a humanized antibody and consequently decreasing potential immunogenicity. However, substitutions can also cause changes of affinity, and significant reductions in affinity are preferably avoided. Positions for substitution within CDRs and amino acids to substitute can also be selected empirically.

### 1. Optional Antibody Engineering

The antibodies of the invention can be modified, or engineered, to alter the amino acid sequences by amino acid substitutions.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution E272Y refers to a variant polypeptide, in this case an Fc variant, in which the glutamic acid at position 272 is replaced with tyrosine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not an amino acid substitution.

As discussed herein, amino acid substitutions can be made to alter the affinity of the CDRs for CoV-S including both increasing and decreasing binding, as is more fully outlined below), as well as to alter additional functional properties of the antibodies. For example, the antibodies may be engineered to include modifications within the Fc region, typically to alter one or more functional properties of the antibody, such as serum half-life, complement fixation, Fc receptor binding, and/or antigen-dependent cellular cytotoxicity. Furthermore, an antibody according to at least some embodiments of the invention may be chemically modified (e.g., one or more chemical moieties can be attached to the antibody) or be modified to alter its glycosylation, again to alter one or more functional properties of the antibody. Such embodiments are described further below. The numbering of residues in the Fc region is that of the EU index of Kabat.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In some embodiments, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In some embodiments, amino acid substitutions can be made in the Fc region, in general for altering binding to FcγR receptors. By “Fc gamma receptor”, “FcγR” or “FcgammaR” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIIb-NA1 and FcγRIIIb-NA2) (Jefferis et al., 2002, Immunol Lett 82:57-65, entirely incorporated by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII-1 (CD16), and FcγRIII-2 (CD16-2), as well as any undiscovered mouse FcγRs or FcγR isoforms or allotypes.

There are a number of useful Fc substitutions that can be made to alter binding to one or more of the FcγR receptors. Substitutions that result in increased binding as well as decreased binding can be useful. For example, it is known that increased binding to FcγRIIIa generally results in increased ADCC (antibody dependent cell-mediated cytotoxicity; the cell-mediated reaction wherein nonspecific cytotoxic cells that express FcγRs recognize bound antibody on a target cell and subsequently cause lysis of the target cell. Similarly, decreased binding to FcγRIIb (an inhibitory receptor) can be beneficial as well in some circumstances. Amino acid substitutions that find use in the present invention include those listed in U.S. Ser. No. 11/124,620 (particularly FIG. 41) and U.S. Pat. No. 6,737,056, both of which are expressly incorporated herein by reference in their entirety and specifically for the variants disclosed therein. Particular variants that find use include, but are not limited to, 236A, 239D, 239E, 332E, 332D, 239D/332E, 267D, 267E, 328F, 267E/328F, 236A/332E, 239D/332E/330Y, 239D, 332E/330L, 299T and 297N.

In some embodiments, the antibodies of the invention are modified to increase its biological half-life. Various approaches are used. For example, one or more of the following mutations can be introduced: T252L, T254S, T256F, as described in U.S. Pat. No. 6,277,375 to Ward. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al. Additional mutations to increase serum half life are disclosed in U.S. Pat. Nos. 8,883,973, 6,737,056 and 7,371,826, and include 428L, 434A, 434S, and 428L/434S.

In some embodiments, the glycosylation of an antibody is modified. For example, an aglycosylated antibody can be made (i.e., the antibody lacks glycosylation). Glycosylation can be altered to, for example, increase the affinity of the antibody for antigen or reduce effector function such as ADCC. Such carbohydrate modifications can be accomplished by, for example, altering one or more sites of glycosylation within the antibody sequence, for example N297. For example, one or more amino acid substitutions can be made that result in elimination of one or more variable region framework glycosylation sites to thereby eliminate glycosylation at that site.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies according to at least some embodiments of the invention to thereby produce an antibody with altered glycosylation. For example, the cell lines Ms704, Ms705, and Ms709 lack the fucosyltransferase gene, FUT8 (α (1,6) fucosyltransferase), such that antibodies expressed in the Ms704, Ms705, and Ms709 cell lines lack fucose on their carbohydrates. The Ms704, Ms705, and Ms709 FUT8 cell lines are created by the targeted disruption of the FUT8 gene in CHO/DG44 cells using two replacement vectors (see U.S. Patent Publication No. 20040110704 by Yamane et al. and Yamane-Ohnuki et al. (2004) Biotechnol Bioeng 87:614-22). As another example, EP 1,176,195 by Hanai et al. describes a cell line with a functionally disrupted FUT8 gene, which encodes a fucosyl transferase, such that antibodies expressed in such a cell line exhibit hypofucosylation by reducing or eliminating the α 1,6 bond-related enzyme. Hanai et al. also describe cell lines which have a low enzyme activity for adding fucose to the N-acetylglucosamine that binds to the Fc region of the antibody or does not have the enzyme activity, for example the rat myeloma cell line YB2/0 (ATCC CRL 1662). PCT Publication WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields, R. L. et al. (2002) J. Biol. Chem. 277:26733-26740). PCT Publication WO 99/54342 by Umana et al. describes cell lines engineered to express glycoprotein-modifying glycosyl transferases (e.g., β(1,4)-N-acetylglucosaminyltransferase II (GnTIII)) such that antibodies expressed in the engineered cell lines exhibit increased bisecting GlcNac structures which results in increased ADCC activity of the antibodies (see also Umana et al. (1999) Nat. Biotech. 17:176-180). Alternatively, the fucose residues of the antibody may be cleaved off using a fucosidase enzyme. For example, the fucosidase α-L-fucosidase removes fucosyl residues from antibodies (Tarentino, A. L. et al. (1975) Biochem. 14:5516-23).

Another modification of the antibodies herein that is contemplated by the invention is pegylation or the addition of other water soluble moieties, typically polymers, e.g., in order to enhance half-life. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies according to at least some embodiments of the invention. See for example, EP 0 154 316 by Nishimura et al. and EP 0 401 384 by Ishikawa et al.

In addition to substitutions made to alter binding affinity to FcγRs and/or FcRn and/or increase in vivo serum half life, additional antibody modifications can be made, as described in further detail below.

In some cases, affinity maturation is done. Amino acid modifications in the CDRs are sometimes referred to as “affinity maturation”. An “affinity matured” antibody is one having one or more alteration(s) in one or more CDRs which results in an improvement in the affinity of the antibody for antigen, compared to a parent antibody which does not possess those alteration(s). In some cases, although rare, it may be desirable to decrease the affinity of an antibody to its antigen, but this is generally not preferred.

In some embodiments, one or more amino acid modifications are made in one or more of the CDRs of the VISG1 antibodies of the invention. In general, only 1 or 2 or 3-amino acids are substituted in any single CDR, and generally no more than from 1, 2, 3, 4, 5, 6, 7, 8 9 or 10 changes are made within a set of CDRs. However, it should be appreciated that any combination of no substitutions, 1, 2 or 3 substitutions in any CDR can be independently and optionally combined with any other substitution.

Affinity maturation can be done to increase the binding affinity of the antibody for the SARS-CoV-2 spike antigen by at least about 10% to 50-100-150% or more, or from 1 to 5 fold as compared to the “parent” antibody. Exemplary affinity matured antibodies will have nanomolar or even picomolar affinities for the SARS-CoV-2 spike antigen. Affinity matured antibodies are produced by known procedures. See, for example, Marks et al., 1992, Biotechnology 10:779-783 that describes affinity maturation by variable heavy chain (VH) and variable light chain (VL) domain shuffling. Random mutagenesis of CDR and/or framework residues is described in: Barbas, et al. 1994, Proc. Nat. Acad. Sci, USA 91:3809-3813; Shier et al., 1995, Gene 169:147-155; Yelton et al., 1995, J. Immunol. 155:1994-2004; Jackson et al., 1995, J. Immunol. 154(7):3310-9; and Hawkins et al, 1992, J. Mol. Biol. 226:889-896, for example.

Alternatively, amino acid modifications can be made in one or more of the CDRs of the antibodies of the invention that are “silent”, e.g. that do not significantly alter the affinity of the antibody for the antigen. These can be made for a number of reasons, including optimizing expression (as can be done for the nucleic acids encoding the antibodies of the invention).

Thus, included within the definition of the CDRs and antibodies of the invention are variant CDRs and antibodies; that is, the antibodies of the invention can include amino acid modifications in one or more of the CDRs of the enumerated antibodies of the invention. In addition, as outlined below, amino acid modifications can also independently and optionally be made in any region outside the CDRs, including framework and constant regions.

### D. SARS-CoV-2 Antibody and Antigen-Binding Fragments

The present invention provides a pharmaceutical composition comprising an antigen anti-CoV-S antibody or antigen-binding fragment thereof. (For convenience, “anti-CoV-S antibodies” and “CoV-S antibodies” are used interchangeably). The anti-CoV-S antibodies of the invention specifically bind CoV-S, and particularly the ECD of the spike protein CoV-S, as depicted in FIG. 12A-PP.

In some embodiments, in order to generate anti-CoV-S antibodies of the present invention, one or more mutations are introduced to the wild type CoV-S sequence. In some embodiments, one or more mutations introduced to CoV-S comprise R691G, R692S, R694S, K995P, V996P, or any combination thereof. In some embodiments, the CoV-S protein of the present invention comprises R691G, R692S, R694S, K995P, and V996P. In some embodiments, the CoV-S protein of the present invention is fused to the T4 fibritin trimerization domain.

In some embodiments, the present invention provides CoV-S antibodies that bind to the RBD within the S1 domain. In some embodiments, the present invention provides CoV-S antibodies that bind to a portion of the S1 domain outside the RBD (i.e., non-RBD S1 domains). In some embodiments, the present invention provides CoV-S antibodies that bind to the S2 domain. In some embodiments, the present invention provides CoV-S antibodies that bind to neither of the S1 (including the RBD) and S2 domains. In some embodiments, the present invention provides CoV-S antibodies that are SARS-CoV2 spike selective.

In some embodiments, the CoV-S antibodies provided herein can be grouped according to reactivity profiles based on binding to the receptor binding domain (RBD) and/or S1 or S2 domains; blocking spike protein binding to the human ACE2 receptor; neutralizing SARS-CoV-2 pseudovirus or SARS-CoV-2 infection of ACE2+ target cells; cross-reactivity with spike proteins from other coronaviruses (e.g., SARS-CoV-1, MERS, HKU1, HCoV-NL63, HCoV-229E, HCoV-OC43); and binding/neutralization of spike proteins from SARS-CoV-2 variants of concern (e.g., B.1.1.7, B.1.351, P.1). Indeed, it is contemplated that the antibodies and CoV-S binding fragments thereof, as described herein, can be used to bind to SARS-CoV-2 variants that are now known as well as those that arise in the future, either for purposes of detection or neutralization (i.e., treatment or prevention of infection). The CoV-S antibodies and binding fragments thereof provided herein may be advantageous in binding and/or neutralizing multiple SARS-CoV-2 variants, while others may be advantageous in specifically targeting the parental virus or one or more specific variants. Exemplary SARS-CoV-2 variants include, without limitation, Alpha (B.1.1.7 and Q lineages), Beta (B.1.351 and descendent lineages), Gamma (P.1 and descendent lineages), Delta (B.1.617.2 and AY lineages), Epsilon (B.1.427 and B.1.429), Eta (B.1.525), Iota (B.1.526), Kappa (B.1.617.1), 1.617.3, Mu (B.1.621, B.1.621.1), Zeta (P.2), and Omicron (B.1.1.529, BA.1, BA.1.1, BA.2, BA.3, BA.4 and BA.5 lineages).

Specific binding for CoV-S or epitope can be exhibited, for example, by an antibody having a K_{D} of at least about 10⁻⁴ M, at least about 10⁻⁵ M, at least about 10⁻⁶ M, at least about 10⁻⁷ M, at least about 10⁻⁸ M, at least about 10⁻⁹ M, or, alternatively, at least about 10⁻¹⁰ M, at least about 10⁻¹¹ M, at least about 10⁻¹² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the CoV-S antigen or epitope.

Also, specific binding for a particular antigen or an epitope can be exhibited, for example, by an antibody having a KA or Ka for CoV-S of at least 20-, 50-, 100-, 500-, 1000-, 5,000-, 10,000- or more times greater for the epitope relative to a control, where KA or Ka refers to an association rate of a particular antibody-antigen interaction.

In some embodiments, the anti-CoV-S antibodies of the invention bind to CoV-S with a KD of 100 nM or less, 50 nM or less, 10 nM or less, or 1 nM or less (that is, higher binding affinity), or 1 pM or less, wherein KD is determined by known methods, e.g. surface plasmon resonance (SPR, e.g. Biacore assays), ELISA, KINEXA, and most typically SPR at 25 or 37° C.

In some embodiments, the antigen-binding portions and variants of the above-identified antibodies retain binding activity that is essentially the same as the binding activity of the whole antibody from which it is derived. By “essentially the same”, it is intended that the antigen-binding portions and variants retain at least 80% (such as at least 85%, or at least 90%, or at least 95%) of the binding affinity (K_{D}) for Cov-S or neutralizing capacity (IC₅₀) for SARS-CoV-2 variants as compared to the parent antibody. In some other embodiments, the antigen-binding portions and variants of the above-identified antibodies retain at least 50% (such as at least 60%, at least 65%, at least 70%, or at least 75%) of the binding activity of the whole antibody (e.g., binding affinity (K_{D}) for Cov-S or neutralizing capacity (IC₅₀) for SARS-CoV-2 variants) from which it is derived.

### 1. Specific Anti-CoV-S Antibodies

The invention provides antigen binding domains, including full length antibodies, which contain a number of specific, enumerated sets of 6 CDRs.

The invention further provides CDRs, variable heavy and light domains as well as full length heavy and light chains as outlined in FIG. 12A-PP including 1-B11-A, 1-L10-A, 2-H7-A, 2-J9-A, 2-O12-A, 2-P2-A, 3-E13-A, 3-P7-A, 4-A15-A, 4-C3-A, 4-K13-A, 4-L4-A, 5-H22-A, 5-P24-A, 6-O12-A, 8-N24-A, 9- J11-A, 9-K4-A, 9-L13-A, 9-P9-A, 10-B11-A, 10-B13-A, 10-L12-A, 10-L24-A, 10-O24-A, 10-O3-A, 4-M3-A, 4-N22-A, 7-B10-A, 8-H5-A, 2-G20-A, 3-E2-A, 4-K16-A, 6-C19-A, 6-L8-A, 7-D7-A, 7-N20-A, 8-A17-A, 8-H3-A, 8-L17-A, 9-F6-A, and 10-I12-A.

As discussed herein, the invention further provides variants of the above components, including variants in the CDRs, as outlined above. In addition, variable heavy chains can be 80%, 90%, 95%, 98% or 99% identical to the “VH” sequences herein, and/or contain from 1, 2, 3, 4, 5, 6, 7, 8, 9, 10 amino acid changes, or more, when Fc variants are used. Variable light chains are provided that can be 80%, 90%, 95%, 98% or 99% identical to the “VL” sequences herein, and/or contain from 1, 2, 3, 4, 5, 6, 7, 8, 9, 10 amino acid changes, or more, when Fc variants are used. Similarly, heavy and light chains are provided that are 80%, 90%, 95%, 98% or 99% identical to the “HC” and “LC” sequences herein, and/or contain from 1, 2, 3, 4, 5, 6, 7, 8, 9, 10 amino acid changes, or more, when Fc variants are used.

Accordingly, the antibodies of the invention comprise CDR amino acid sequences selected from the group consisting of (a) sequences as listed herein; (b) sequences that differ from those CDR amino acid sequences specified in (a) by 1, 2, 3, 4, 5, 6, 7, 8, 9, 10 or more amino acid substitutions; (c) amino acid sequences having 90% or greater, 95% or greater, 98% or greater, or 99% or greater sequence identity to the sequences specified in (a) or (b); (d) a polypeptide having an amino acid sequence encoded by a polynucleotide having a nucleic acid sequence encoding the amino acids as listed herein.

Additionally, included in the definition of CoV-S antibodies are antibodies that share identity to the CoV-S antibodies enumerated herein. That is, in certain embodiments, an anti-CoV-S antibody according to the invention comprises heavy and light chain variable regions comprising amino acid sequences that are homologous to isolated anti-CoV-S amino acid sequences of exemplary anti-CoV-S immune molecules, respectively, wherein the antibodies retain the desired functional properties of the parent anti-CoV-S antibodies. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The antibodies of the invention include those antibodies having the identical CDRs but differing in the variable domain (or entire heavy or light chain). For example, antibodies include those with CDRs identical to those shown in FIG. 12A-PP but whose identity along the variable region can be lower, for example 95 or 98% percent identical.

The present invention provides not only the enumerated antibodies but additional antibodies that compete with the enumerated antibodies to specifically bind to CoV-S. Additional antibodies that compete with the enumerated antibodies are generated, as is known in the art and generally outlined below. Competitive binding studies can be done as is known in the art, generally using SPR/Biacore® binding assays, as well as ELISA and cell-based assays.

Methods of generating antibodies as well as subsequent screening assays are well-known in the art, such as those outlined in the examples. In some embodiments, anti-CoV-S antibodies are generated by traditional methods such as immunizing mice (sometimes using DNA immunization), followed by screening against CoV-S and hybridoma generation, with antibody purification and recovery.

### E. Formulations of Anti-CoV-S Antibodies

The therapeutic compositions used in the practice of the present invention can be formulated into pharmaceutical compositions comprising a carrier suitable for the desired delivery method. Suitable carriers include any material that when combined with the therapeutic composition retains the anti-tumor function of the therapeutic composition and is generally non-reactive with the patient's immune system. Examples include, but are not limited to, any of a number of standard pharmaceutical carriers such as sterile phosphate buffered saline solutions, bacteriostatic water, and the like (see, generally, Remington's Pharmaceutical Sciences 16th Edition, A. Osal., Ed., 1980). Acceptable carriers, excipients, or stabilizers are nontoxic to recipients at the dosages and concentrations employed, and include buffers such as phosphate, citrate, acetate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; sweeteners and other flavoring agents; fillers such as microcrystalline cellulose, lactose, corn and other starches; binding agents; additives; coloring agents; salt-forming counter-ions such as sodium; metal complexes (e.g. Zn-protein complexes); and/or non-ionic surfactants such as TWEEN™, PLURONICS™ or polyethylene glycol (PEG).

In some embodiments, the pharmaceutical composition that comprises the antibodies of the invention may be in a water-soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Exemplary ones are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine. The formulations to be used for in vivo administration are preferably sterile. This is readily accomplished by filtration through sterile filtration membranes or other methods.

Administration of the pharmaceutical composition comprising antibodies of the present invention, for example in the form of a sterile aqueous solution, may be done in a variety of ways, including, but not limited to subcutaneously, intravenously, and intranasally. Subcutaneous administration may be done in some circumstances because the patient may self-administer the pharmaceutical composition. Many protein therapeutics are not sufficiently potent to allow for formulation of a therapeutically effective dose in the maximum acceptable volume for subcutaneous administration. This problem may be addressed in part by the use of protein formulations comprising arginine-HCl, histidine, and polysorbate (see WO04091658). Fc polypeptides of the present invention may be more amenable to subcutaneous administration due to, for example, increased potency, improved serum half-life, or enhanced solubility.

As is known in the art, protein therapeutics are often delivered by IV infusion or bolus. The antibodies of the present invention may also be delivered using such methods. For example, administration may be by intravenous infusion with 0.9% sodium chloride as an infusion vehicle.

In addition, any of a number of delivery systems are known in the art and may be used to administer the Fc variants of the present invention. Examples include, but are not limited to, encapsulation in liposomes, microparticles, microspheres (e.g. PLA/PGA microspheres), and the like. Alternatively, an implant of a porous, non-porous, or gelatinous material, including membranes or fibers, may be used. Sustained release systems may comprise a polymeric material or matrix such as polyesters, hydrogels, poly(vinylalcohol), polylactides, copolymers of L-glutamic acid and ethyl-L-gutamate, ethylene-vinyl acetate, lactic acid-glycolic acid copolymers such as the LUPRON DEPOT©, and poly-D-(−)-3-hydroxybutyric acid. The antibodies disclosed herein may also be formulated as immunoliposomes. A liposome is a small vesicle comprising various types of lipids, phospholipids and/or surfactant that is useful for delivery of a therapeutic agent to a mammal. Liposomes containing the antibody are prepared by methods known in the art, such as described in Epstein et al., 1985, Proc Natl Acad Sci USA, 82:3688; Hwang et al., 1980, Proc Natl Acad Sci USA, 77:4030; U.S. Pat. Nos. 4,485,045; 4,544,545; and PCT WO 97/38731. Liposomes with enhanced circulation time are disclosed in U.S. Pat. No. 5,013,556. The components of the liposome are commonly arranged in a bilayer formation, similar to the lipid arrangement of biological membranes. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. A chemotherapeutic agent or other therapeutically active agent is optionally contained within the liposome (Gabizon et al., 1989, J National Cancer Inst 81:1484).

The antibodies may also be entrapped in microcapsules prepared by methods including but not limited to coacervation techniques, interfacial polymerization (for example using hydroxymethylcellulose or gelatin-microcapsules, or poly-(methylmethacylate) microcapsules), colloidal drug delivery systems (for example, liposomes, albumin microspheres, microemulsions, nano-particles and nanocapsules), and macroemulsions. Such techniques are disclosed in Remington's Pharmaceutical Sciences 16th edition, Osol, A. Ed., 1980. Sustained-release preparations may be prepared. Suitable examples of sustained-release preparations include semipermeable matrices of solid hydrophobic polymer, which matrices are in the form of shaped articles, e.g. films, or microcapsules. Examples of sustained-release matrices include polyesters, hydrogels (for example poly(2-hydroxyethyl-methacrylate), or poly(vinylalcohol)), polylactides (U.S. Pat. No. 3,773,919), copolymers of L-glutamic acid and gamma ethyl-L-glutamate, non-degradable ethylene-vinyl acetate, degradable lactic acid-glycolic acid copolymers such as the LUPRON DEPOT© (which are injectable microspheres composed of lactic acid-glycolic acid copolymer and leuprolide acetate), poly-D-(−)-3-hydroxybutyric acid, and ProLease© (commercially available from Alkermes), which is a microsphere-based delivery system composed of the desired bioactive molecule incorporated into a matrix of poly-DL-lactide-co-glycolide (PLG).

The dosing amounts and frequencies of administration are, in some embodiments, selected to be therapeutically or prophylactically effective. As is known in the art, adjustments for protein degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

The concentration of the antibody in the formulation may vary from about 0.1 to 100 weight %. In some embodiments, the concentration of the Fc variant is in the range of 0.003 to 1.0 molar. In order to treat a patient, a therapeutically effective dose of the Fc variant of the present invention may be administered. By “therapeutically effective dose” herein is meant a dose that produces the effects for which it is administered. The exact dose will depend on the purpose of the treatment, and will be ascertainable by one skilled in the art using known techniques. Dosages may range from about 0.0001 to 100 mg/kg of body weight or greater, for example about 0.1, 1, 10, or 50 mg/kg of body weight, and in an exemplary embodiment, from about 1 to 10 mg/kg.

The therapeutic compositions used in the practice of the foregoing methods can be formulated into pharmaceutical compositions comprising a carrier suitable for the desired delivery method. Suitable carriers include any material that when combined with the therapeutic composition retains the anti-tumor function of the therapeutic composition and is generally non-reactive with the patient's immune system. Examples include, but are not limited to, any of a number of standard pharmaceutical carriers such as sterile phosphate buffered saline solutions, bacteriostatic water, and the like (see, generally, Remington's Pharmaceutical Sciences 16th Edition, A. Osal., Ed., 1980). Acceptable carriers, excipients, or stabilizers are nontoxic to recipients at the dosages and concentrations employed, and include buffers such as phosphate, citrate, acetate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; sweeteners and other flavoring agents; fillers such as microcrystalline cellulose, lactose, corn and other starches; binding agents; additives; coloring agents; salt-forming counter-ions such as sodium; metal complexes (e.g. Zn-protein complexes); and/or non-ionic surfactants such as TWEEN™, PLURONICS™ or polyethylene glycol (PEG).

### F. Nucleic Acids, Expression Vectors, Host Cells

In some embodiments, the present invention provides nucleic acids encoding the antibodies or antigen-binding domains as described herein. As will be appreciated by those in the art, the protein sequences depicted herein can be encoded by any number of possible nucleic acid sequences, due to the degeneracy of the genetic code. In some embodiments, the nucleic acid molecules are DNA. In some embodiments, the nucleic acid molecules are RNA.

The nucleic acid compositions that encode the CoV-S antibodies will depend on the format of the antibody. In exemplary embodiments, tetrameric antibodies containing two heavy chains and two light chains are encoded by two different nucleic acids, one encoding the heavy chain and one encoding the light chain. These can be put into a single expression vector or two expression vectors, as is known in the art, transformed into host cells, where they are expressed to form the antibodies of the invention. In some embodiments, for example when scFv constructs are used, a single nucleic acid encoding the variable heavy chain-linker-variable light chain is generally used, which can be inserted into an expression vector for transformation into host cells. The nucleic acids can be put into expression vectors that contain the appropriate transcriptional and translational control sequences, including, but not limited to, signal and secretion sequences, regulatory sequences, promoters, origins of replication, selection genes, etc.

Exemplary mammalian host cells for expressing the recombinant antibodies according to at least some embodiments of the invention include Chinese Hamster Ovary (CHO cells), PER.C6, HEK293 and others as is known in the art.

The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art.

To create a scFv gene, the VH- and VL-encoding DNA fragments are operatively linked to another fragment encoding a flexible linker, e.g., encoding the amino acid sequence (Gly4-Ser)3, such that the VH and VL sequences can be expressed as a contiguous single-chain protein, with the VL and VH regions joined by the flexible linker (see e.g., Bird et al. (1988) Science 242:423-426; Huston et al. (1988) Proc. Natl. Acad. Sci. USA 85:5879-5883; McCafferty et al., (1990) Nature 348:552-554).

### G. Therapeutic Application of the Antibodies or Antigen-Binding Fragments

The present invention provides methods for treating or preventing viral infection (e.g., coronavirus infection) by administering a therapeutically effective amount of anti-CoV-S spike antigen-binding protein, e.g., antibody or antigen-binding fragment, (e.g., of FIG. 12A-12PP) to a subject (e.g., a human) in need of such treatment or prevention.

Coronavirus infection may be treated or prevented, in a subject, by administering an antibody or antigen-binding fragment of the present invention to a subject.

An effective or therapeutically effective dose of anti-CoV-S antigen-binding protein, e.g., antibody or antigen-binding fragment (e.g., of FIG. 12A-12PP), for treating or preventing a viral infection refers to the amount of the antibody or fragment sufficient to alleviate one or more signs and/or symptoms of the infection in the treated subject, whether by inducing the regression or elimination of such signs and/or symptoms or by inhibiting the progression of such signs and/or symptoms. The dose amount may vary depending upon the age and the size of a subject to be administered, target disease, conditions, route of administration, and the like. In an embodiment of the invention, an effective or therapeutically effective dose of antibody or antigen-binding fragment thereof of the present invention, for treating or preventing viral infection, e.g., in an adult human subject, is about 0.01 to about 200 mg/kg, e.g., up to about 150 mg/kg. In an embodiment of the invention, the dosage is up to about 10.8 or 11 grams (e.g., about 1, 2, 3, 4, 5, 6, 7, 8, 9, 10 or 11 grams). Depending on the severity of the infection, the frequency and the duration of the treatment can be adjusted. In certain embodiments, the antigen-binding protein of the present invention can be administered at an initial dose, followed by one or more secondary doses. In certain embodiments, the initial dose may be followed by administration of a second or a plurality of subsequent doses of antibody or antigen-binding fragment thereof in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

In some embodiments, the method of preventing viral infection provided herein comprises prophylactically administering an antibody or antigen-binding fragment of the present invention (e.g., of FIG. 12A-12PP), to a subject who is at risk of viral infection so as to prevent such infection. Passive antibody-based immunoprophylaxis has proven an effective strategy for preventing subject from viral infection. See e.g., Berry et al., Passive broad-spectrum influenza immunoprophylaxis. Influenza Res Treat. 2014; 2014:267594. Epub 2014 Sep. 22; and Jianqiang et al., Passive immune neutralization strategies for prevention and control of influenza A infections, Immunotherapy. 2012 February; 4(2): 175-186; Prabhu et al., Antivir Ther. 2009; 14(7):911-21, Prophylactic and therapeutic efficacy of a chimeric monoclonal antibody specific for H5 hemagglutinin against lethal H5N1 influenza. “Prevent” or “preventing” means to administer an antibody or antigen-binding fragment of the present invention (e.g., of FIG. 12A-12PP), to a subject to inhibit the manifestation of a disease or infection (e.g., viral infection) in the body of a subject, for which the antigen-binding protein is effective when administered to the subject at an effective or therapeutically effective amount or dose (as discussed herein).

In an embodiment of the invention, a sign or symptom of a viral infection in a subject is survival or proliferation of virus in the body of the subject, e.g., as determined by viral titer assay (e.g., coronavirus propagation in embryonated chicken eggs or coronavirus spike protein assay). Other signs and symptoms of viral infection are discussed herein.

As noted above, in some embodiments the subject may be a non-human animal, and the antigen-binding proteins (e.g., antibodies and antigen-binding fragments) discussed herein may be used in a veterinary context to treat and/or prevent disease in the non-human animals (e.g., cats, dogs, pigs, cows, horses, goats, rabbits, sheep, and the like).

In some embodiments, the present invention provides a method for treating or preventing viral infection (e.g., coronavirus infection) or for inducing the regression or elimination or inhibiting the progression of at least one sign or symptom of viral infection such as: fever or feeling feverish/chills; cough; sore throat; runny or stuffy nose; sneezing; muscle or body aches; headaches; fatigue (tiredness); vomiting; diarrhea; respiratory tract infection; chest discomfort; shortness of breath; bronchitis; and/or pneumonia, which sign or symptom is secondary to viral infection, in a subject in need thereof (e.g., a human), by administering a therapeutically effective amount of antibody or antigen-binding fragment (e.g., of FIG. 12A-12PP) to the subject, for example, by injection of the protein into the body of the subject.

### H. Diagnostic Application of the Antibodies or Antigen-Binding Fragments

In some embodiments, the antibody or antigen-binding fragment thereof of the present invention (e.g., of FIG. 12A-12PP), may be used to detect and/or measure SARS-Cov-2 in a sample. Exemplary assays for CoV-S may include, e.g., contacting a sample with an SARS-CoV-2 antibody of the invention, wherein the antibody is labeled with a detectable label or reporter molecule or used as a capture ligand to selectively isolate CoV-S from samples. The presence of a CoV-S antibody complexed with CoV-S indicates the presence of the SARS-Cov-2 virus in the sample. Alternatively, an unlabeled SARS-CoV-2 antibody can be used in combination with a secondary antibody which is itself detectably labeled. The detectable label or reporter molecule can be a radioisotope, such as 3H, 14C, 32P 35S, or 125I; a fluorescent or chemiluminescent moiety such as fluorescein isothiocyanate, or rhodamine; or an enzyme such as alkaline phosphatase, β-galactosidase, horseradish peroxidase, or luciferase. Specific exemplary assays that can be used to detect or measure CoV-S in a sample include neutralization assays, enzyme-linked immunosorbent assay (ELISA), radioimmunoassay (RIA), and fluorescence-activated cell sorting (FACS). Thus, the present invention includes a method for detecting the presence of spike protein polypeptide in a sample comprising contacting the sample with a CoV-S antibody and detecting the presence of the antibody wherein the presence of the complex indicates the presence of the SARS-CoV-2 virus in the sample.

In some embodiments, the CoV-S antibodies of the invention (e.g., of FIG. 12A-12PP) may be used in a Western blot or immune-protein blot procedure for detecting the presence of SARS-CoV-2 in a sample.

In some embodiments, the CoV-S antibodies disclosed herein may also be used for immunohistochemistry.

Any of a variety of suitable biological (patient) samples can be used for diagnostic detection of SARS-Cov-2 in a sample. Exemplary biological samples include, without limitation, bronchoalveolar fluid, nasopharyngeal swabs, sputum, blood, feces and anal swabs, and urine.

### EXAMPLES

The following examples are provided to illustrate certain embodiments of the invention. The invention is not limited to these examples and the full scope of the invention is reflected in the claims appended hereto.

### Example 1

As part of the ongoing effort to combat the SARS-CoV-2 pandemic, a diverse panel of 42 antibodies against the spike protein was discovered. Strains of wild type (WT) mice in the PentaMice® platform were immunized with adjuvanted spike protein and monoclonal antibodies were obtained using an optimized hybridoma-based antibody recovery workflow. Based on extensive screening assays, the discovered antibodies display a wide range of binding specificities and functional properties. The spike antibodies can be grouped according to reactivity profiles based on binding to the receptor binding domain (RBD) and/or S1 or S2 domains; blocking spike protein binding to the human ACE2 receptor; neutralizing SARS-CoV-2 pseudovirus or SARS-CoV-2 infection of ACE2+ target cells; cross-reactivity with spike proteins from other coronaviruses (SARS-CoV-1, MERS, HKU1, HCoV-NL63, HCoV-229E, HCoV-OC43); and binding/neutralization of spike proteins from SARS-CoV-2 variants of concern (B.1.1.7, B.1.351, P.1). The various profiles are consistent with antibody therapeutics, prevention of SARS-CoV-2 infection, and selective SARS-CoV-2 detection diagnostics.

### Methods

Immunizations and antibody recovery: The SARS-CoV-2 spike protein extracellular domain (Gene ID/Reference: MN996527.1 (GenBank), ECD (23-1222), WIV02 isolate) was expressed using the TunaCHO™ manufacturing platform. The furin-recognition site RRAR was mutated to GSAS. KV was mutated to PP to stabilize the protein in a prefusion conformation. A T4 fibritin trimerization domain was added to the carboxyl-terminal domain to facilitate trimerization. These features are indicated in FIG. 1 (SEQ ID NO: 1). Strains of wild type (WT) mice in the PentaMice© platform were immunized with adjuvanted spike protein. PentaMice are a proprietary set of WT mice generated via in-house breeding that comprise 5 strains of F1 and outbred WT mice and cover 9 distinct major histocompatibility complex (MHC) class II (I-A, I-E) haplotypes (b, d, g7, k, q, s, u, v, and mixed). When in-life plasma titers indicated that a strong anti-spike protein humoral immune response was achieved, the animals were euthanized and lymphocytes harvested and fused with a myeloma partner via electrofusion to generate hybridomas. The hybridomas were plated into ten 384-well plates and supernatants were screened for reactivity against SARS-CoV-2 spike protein by ELISA. Candidate parental hybridomas were subjected to limiting dilution cloning to generate monoclonal hybridomas. Variable heavy and light chain sequences were determined for 42 monoclonal antibodies. Purified antibodies were generated and assessed for various binding and functional characteristics. Three mAbs were reformatted and expressed as human Fc IgG2 chimeras (10-B13-A, 10-O24-A, and 6-O12-A). Three mAbs (4-C3-A, 5-P24-A, and 2-J9-A) were expressed recombinantly as mouse Fc IgG2b antibodies. The recombinantly expressed mAbs retained their binding properties.

ELISA: mAb binding reactivity was assessed by ELISA against the following antigens: SARS-CoV-2 (WIV02 isolate) spike protein (see FIG. 1); SARS-CoV-2 S1 domain (sequence contained within FIG. 1); SARS-CoV-2 S2 domain (sequence contained within FIG. 1); SARS-CoV-2 receptor binding domain (RBD, sequence contained within FIG. 1); SARS-CoV-1 spike protein; MERS spike protein; HKU1 spike protein; HCoV-NL63 spike protein; HCoV229E spike protein; HCoV-OC43 spike protein; SARS-CoV-2 B.1.1.7 spike protein; SARS-CoV-2 B.1.351 spike protein; SARS-CoV-2 P.1 spike protein; BVP (baculovirus particles, non-specific binding); ICOS-His (irrelevant His-tagged negative control protein). The MERS spike protein corresponds to the sequence reported at GenBank AFY13307.1, UniProtKB K9N5Q8, which are hereby incorporated by reference in their entirety; the SARS-CoV-1 spike protein corresponds to the sequence reported at GenBank AAP13441.1, UniProtKB P59594, which are hereby incorporated by reference in their entirety); the HKU1 spike protein corresponds to the sequence reported at Genbank ADN03339.1, UniProtKB E0YJ44, which are hereby incorporated by reference in their entirety); the HCoV-NL63 spike protein corresponds to the sequence reported at UniProtKB Q6Q1S2 (residues 24-1294), which is hereby incorporated by reference in its entirety; the HCoV229E spike protein corresponds to the sequence reported at UniProtKB P15423 (residues 17-1103), which is hereby incorporated by reference in its entirety; and the HCoV-OC43 spike protein corresponds to the sequence reported at UniProtKB Q696P8, GenBank: AAT84354.1 (residues 1-1263), which are hereby incorporated by reference in their entirety. The SARS-CoV-2 B.1.1.7 spike protein, SARS-CoV-2 B.1.351 spike protein, and SARS-CoV-2 P.1 spike protein variants were formed by mutating the sequence corresponding to the sequence reported at GenBank MN996527.1/UniProtKB J2778 with the mutations identified on the CDC website for those spike protein variants. ELISA plates were coated with antigen (1-10 ug/mL) and blocked with 3% bovine serum albumin (BSA). Various dilutions of antibodies are added to the coated blocked plates and incubated 1 hour at room temperature and then washed. Anti-mouse IgG-horse radish peroxidase (HRP) in blocking buffer is added to the wells and incubated 1 hour at room temperature and washed. Pre-mixed SuperSignal ELISA Pico substrate (Thermo) solution is added to each well and bound protein is detected using Molecular Devices SpectraMax M3 luminometer and Softmax Pro Version 6.2 within 15 minutes of adding substrate.

ELISA Neutralization Assay: Human angiotensin-converting enzyme 2 (ACE2) is an entry receptor for SARS-CoV-2 and SARS-CoV-1 via binding to the RBD domain of the viral spike protein. An ELISA was developed to evaluate the ability of spike-binding mAbs to neutralize the interaction of the SARS-CoV-2 S protein RBD with the ACE2 receptor. The neutralizing antibody assay is similar to a COVID-19 Spike-ACE2 binding assay kit II for COVID-19 drug and antibody screening (Ray Biotech, Inc., Peachtree Corners, GA) and is described in the literature (Byrnes et al. 2020; Tai et al. 2020). In this assay, a 384-well ELISA plate is coated with recombinant huACE2 protein-human fragment crystallizable region (Fc); (5 ug/mL), blocked with 3% BSA for 1 hour at room temperature. Various dilutions of antibodies are pre-mixed with histidine-tagged spike proteins (either SARS-CoV-2 WT WIV02 spike trimer (FIG. 1); SARS-CoV-2 B.1.1.7 spike trimer variant; SARS-CoV-2 B.1.351 spike trimer variant; SARS-CoV-2 P.1 spike trimer variant; or SARS-CoV-1 spike trimer; all 1 ug/mL) for at least 15 minutes at room temperature and then added to the 384-well plate and incubated at room temperature for 1 hour. After incubation, plates are washed 4 times, rotated 180 degrees, and washed an additional 4 times. Bound protein is detected following incubation with anti-His-HRP antibody for 1 hour at room temperature. Pre-mixed SuperSignal ELISA Pico substrate (Thermo) solution is added to each well and bound protein is detected using Molecular Devices SpectraMax M3 luminometer and Softmax Pro Version 6.2 within 15 minutes of adding substrate.

SARS-CoV-2 and SARS-CoV-1 pseudovirus infection of ACE2+TMPRSS2+ target cells: Targeted 293T cells were transfected with pcDNA3.1(+)-ACE2 and pCSDest-TMPRSS2 for 6 h. The cells were then trypsinized and seeded 1×10⁵ cells/well in DMEM complete into 96-well plates (100 μL/well) then incubated for 16 hours at 37° C. and 5% CO₂. The antibodies were 3-fold serially diluted in a pseudovirus/buffer mixture. Based on the antibody concentration, 1 M HEPES buffer was used to dilute the pseudovirus to the correct percent buffer concentration in all wells except the first. Virions were incubated with the test samples at room temperature for 1 h, and then added to the target cells in 96-well plates. Plates were incubated for 48 hours and degree of viral infection was determined by luminescence using the neolite reporter gene assay system (PerkinElmer). All error bars represent S.D. from three replicates.

BSL3 SARS-CoV-2 infection of Vero E6 Cells: Vero E6 cells were seeded 5×10⁵ cells/well in DMEM complete into 12-well plates (1 mL/well) then incubated for 16 hours at 37° C. and 5% CO₂. The plaque reduction neutralization test (PRNT) was performed using a clinical isolate of SARS-CoV-2 (SARS-CoV-2, Isolate USA-WA1/2020) from BEI Resources. 3-fold serial dilutions of mAbs were added to the same volume of SARS-CoV-2 (final MOI=0.0001) and incubated for 1 hour at 37° C. The mixture was added to the monolayer of Vero E6 cells and incubated for 1 hour at 37° C. and 5% CO₂. The mixture was removed, 1 mL of 1.25% (w/v) Avicel-591 in 2×MEM supplied with 4% (v/v) FBS was added onto infected cells. Plates were incubated 48 hours at 37° C. and 5% CO₂. After the 48-hour incubation, the plates were fixed with 10% (v/v) formaldehyde and stained with 1% (w/v) crystal violet to visualize the plaques. All experiments were performed in a Biosafety level 3 facility.

Z² Developability score: The Z² developability score is an assessment of certain theoretical developability issues via sequence-based identification of six common potential liability parameters [unpaired cysteine (40.0), N-linked glycosylation (13.3), deamidation (6.3), pyroglutamate formation (5.7), isomerization (3.6), oxidation in CDRs (1.5))], each of which is weighted based on its frequency in a set of 20 FDA-approved, manufactured, and marketed monoclonal antibodies (Secukinumab, Cetuximab, Infliximab, Rituximab, Brentuximab vendotin, Trastuzumab, Vedolizumab, Ipilimumab, Panitumumab, Bevacizumab, Duplimab, Atezolizumab, Omalizumab, Pembrolizumab, Nivolumab, Ocrelizumab, Alemtuzumab, Ranibizumab, Usekinumab, Pertuzumab). Antibodies that lack any theoretical sequence-based liabilities have a Z² score of zero (e.g. Pertuzumab). Antibodies with a higher score have one or more theoretical developability issues (e.g. Secukinumab has an unpaired cysteine; an isomerization potential; and an oxidation potential; total Z²=45.1).

Binding kinetics via surface plasma resonance (SPR): Binding experiments were performed on Carterra LSA. Candidate antibodies (ligands) were diluted to 10 μg/mL in 10 mM NaOAc pH 4.5 containing 0.01% Tween-20 and coupled to a HC30M chip via sulpho-NHS/EDC coupling chemistry and blocked with ethanolamine. Buffer exchange of antigen SARS-CoV-2 Spike Protein RBD were performed using Zeba column prior to Carterra analysis. SARS-CoV-2 (2019-nCoV) Spike RBD-His Recombinant Protein, Lot TP31549F S Protein RBD(319-591)-His10. Original formulation: 50 mM Tris pH 7.5, 150 mM NaCl, 0.05% NaN3. Serial dilutions (1000 nM start, 1:3 dilution, 8 points) of RBD were injected for kinetic constant determination. At the end of each cycle, the chip was regenerated with 10 mM Glycine pH 2.0 to remove bound antigen. Kinetics analysis was performed using Carterra kinetics software.

Dendrogram: A phylogenetic dendrogram for 42 spike-binding mAb protein sequences was built by MUSCLE alignment and Neighbor-joining using Geneious software. The heavy chain and light chain sequences for each mAb were concatenated into one sequence (separated by a 4×GGGS linker). The confidence (%) after resampling against the consensus tree is displayed at each node. The resample method is bootstrap. The number of resampling replicates is 100.

FIG. 1 demonstrates the SARS-CoV-2 prefusion stabilized trimer protein immunogen. The SARS-CoV-2 spike protein extracellular domain (Gene ID/Reference: MN996527.1 (GenBank), ECD (23-1222), WIV02 isolate) was expressed using the TunaCHO℠ manufacturing platform. The furin-recognition site RRAR was mutated to GSAS. KV was mutated to PP to stabilize the protein in a prefusion conformation. A T4 fibritin trimerization domain was added to the carboxyl-terminus to facilitate trimerization.

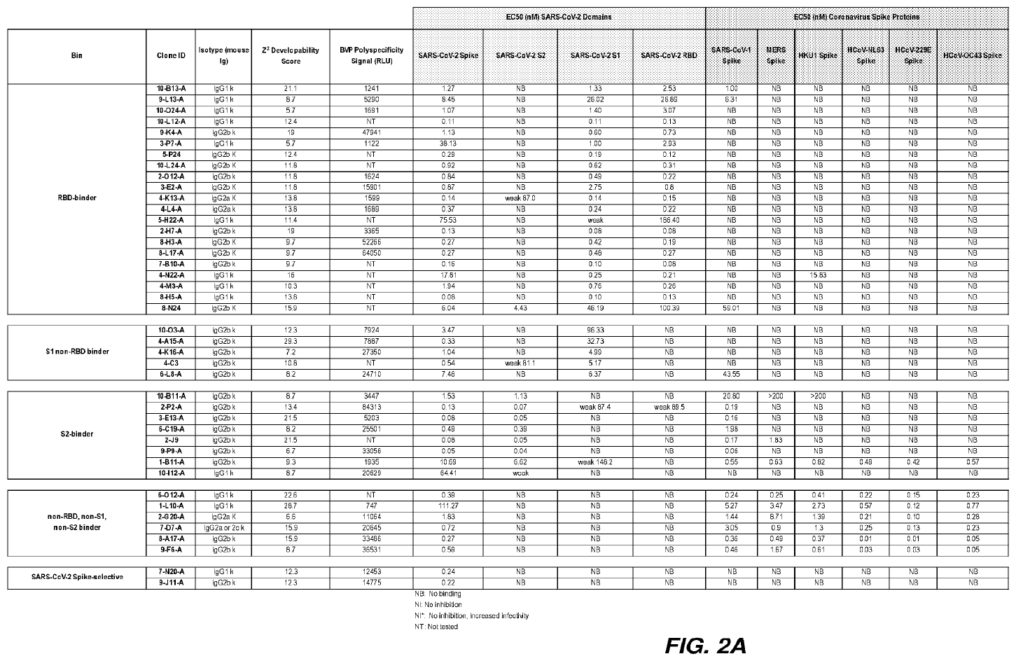

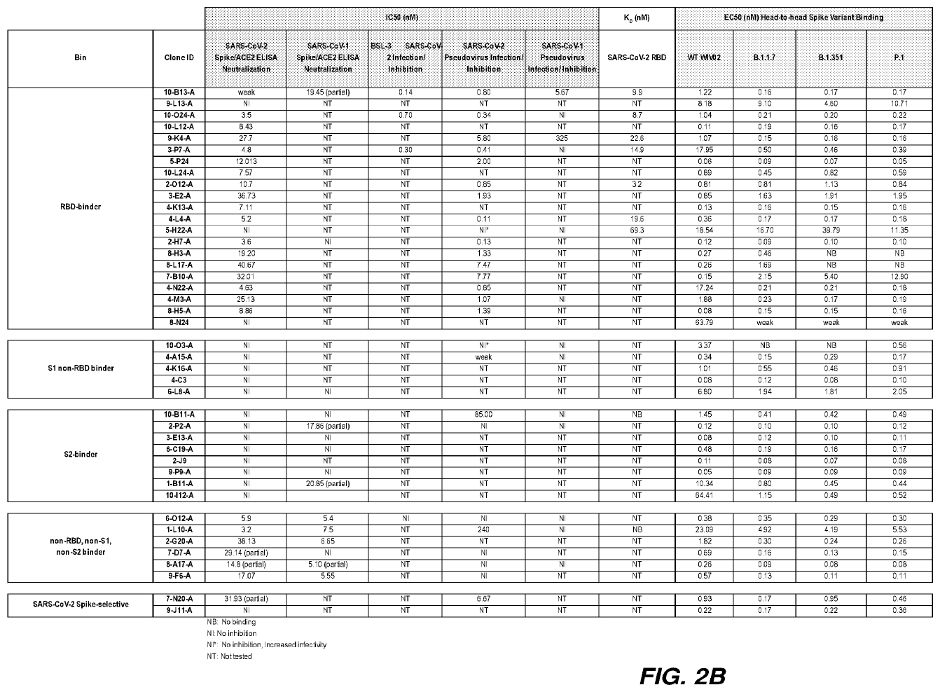

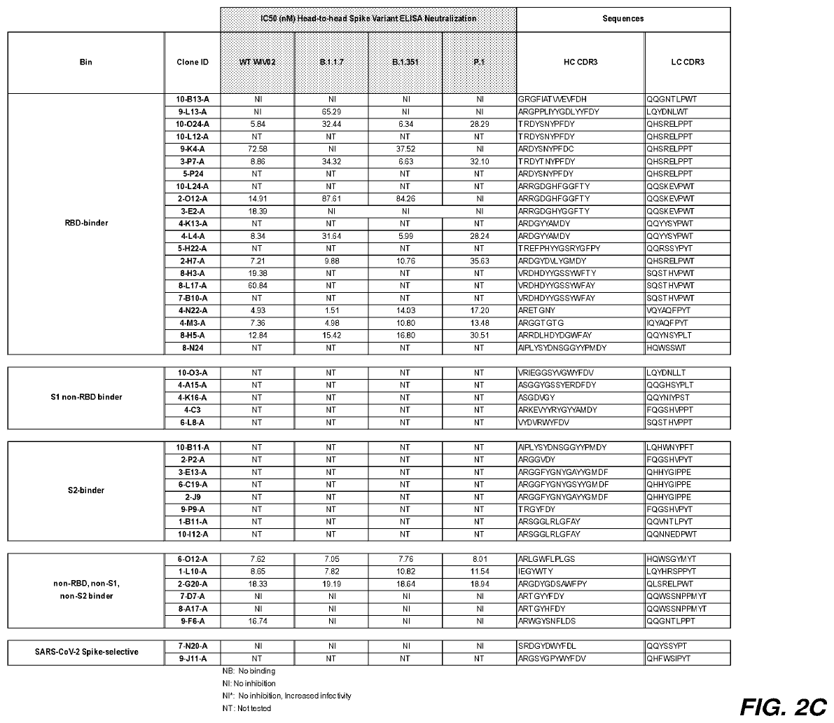

FIG. 2 provides a comprehensive analytic summary of 42 SARS-CoV-2 spike binding mAbs. From left-to-right, the chart provides the heavy chain and light chain isotype; Z2 developability score; BVP polyspecificity ELISA signal; SARS-CoV-2 spike trimer, S2, S1, and RBD domain EC50 ELISA values; SARS-CoV-1 spike trimer, MERS spike trimer, HKU1 spike trimer, HCoV-NL63 spike trimer, HCoV-229E spike trimer, HCoV-OC43 spike trimer EC50 ELISA values; IC50 neutralization values for SARS-CoV-2 spike/ACE2 ELISA binding inhibition, SARS-CoV-1 spike/ACE2 ELISA binding inhibition, BSL-3 SARS-CoV-2 infection/inhibition, SARS-CoV-2 pseudovirus infection/inhibition, SARS-CoV-1 pseudovirus infection/inhibition; SARS-CoV-2 RBD binding KD; SARS-CoV-2 WIV02 WT spike trimer, SARS-CoV-2 B.1.1.7 spike trimer variant; SARS-CoV-2 B.1.351 spike trimer variant; SARS-CoV-2 P.1 spike trimer variant EC50 ELISA values; IC50 neutralization values for SARS-CoV-2 WIV02 WT spike trimer/ACE2 ELISA binding inhibition, SARS-CoV-2 B.1.1.7 spike trimer variant/ACE2 ELISA binding inhibition; SARS-CoV-2 B.1.351 spike trimer variant/ACE2 ELISA binding inhibition; SARS-CoV-2 P.1 spike trimer variant/ACE2 ELISA binding inhibition; heavy chain (HC) and light chain (LC) complementarity determining region 3 (CDR3) domain amino acid sequences.

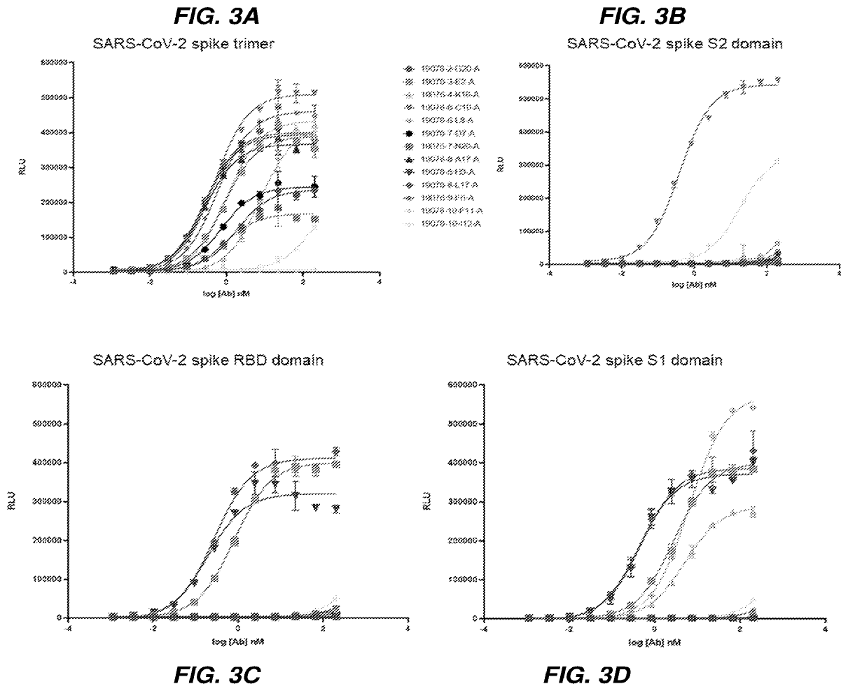

FIGS. 3A-3D illustrate EC50 ELISA binding curves for selected SARS-CoV-2 spike-binding mAbs against spike trimer, S2 domain, RBD domain, and S1 domain, respectively. 10-F11-A is included as a negative control mAb that does not bind to SARS-CoV-2 spike protein.

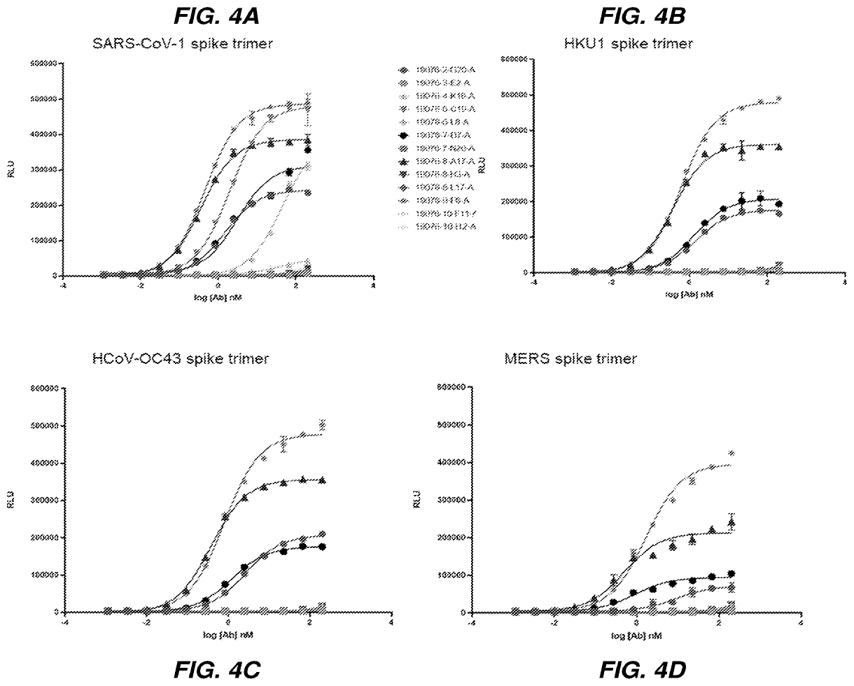

FIGS. 4A-4D illustrate EC50 ELISA binding curves for selected SARS-CoV-2 spike-binding mAbs against spike trimers from SARS-CoV-1, HKU1, HCOV-OC43, and MERS, respectively. 10-F11-A is included as a negative control mAb that does not bind to SARS-CoV-2 spike protein.

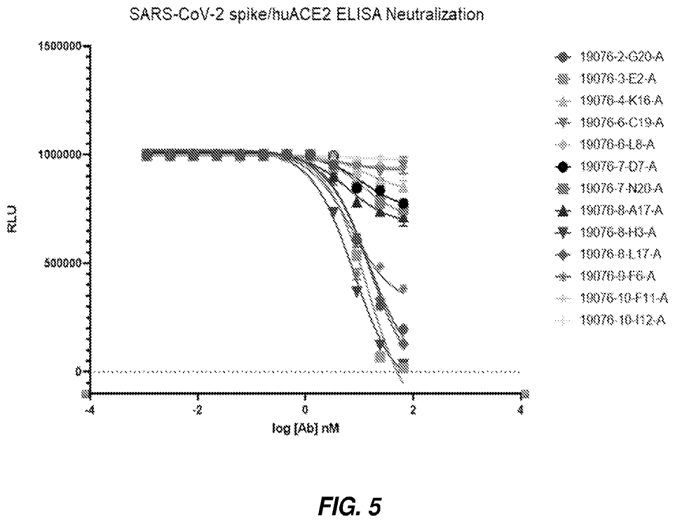

FIG. 5 illustrates IC50 ELISA neutralization curves for selected SARS-CoV-2 spike-binding mAbs inhibiting the binding of SARS-CoV-2 spike trimer to huACE2. 10-F11-A is included as a negative control mAb that does not bind/neutralize SARS-CoV-2 spike protein.

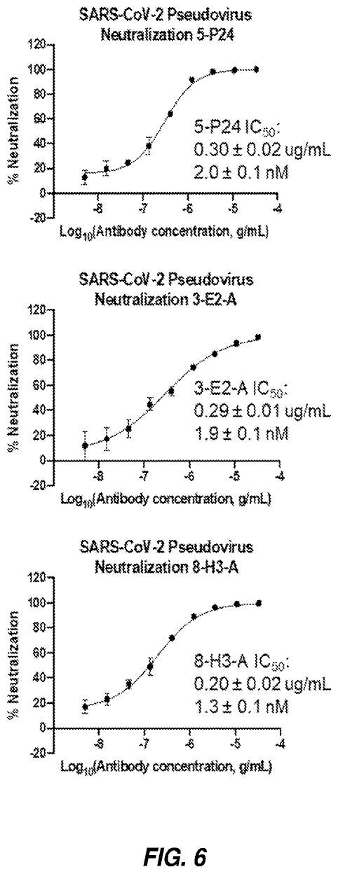

FIG. 6 shows IC50 titration of 5-P24-A, 3-E2-A, and 8-H3-A in SARS-CoV-2 pseudovirus ACE2+TMPRSS2+ target cell infection assay. IC50 values were determined by fitting the dose-response curves with four-parameter logistic regression in Prism GraphPad (version 8.1.2) All data was normalized to pseudovirus alone. All error bars represent S.D. from three replicates. All error bars represent S.D. from three replicates.

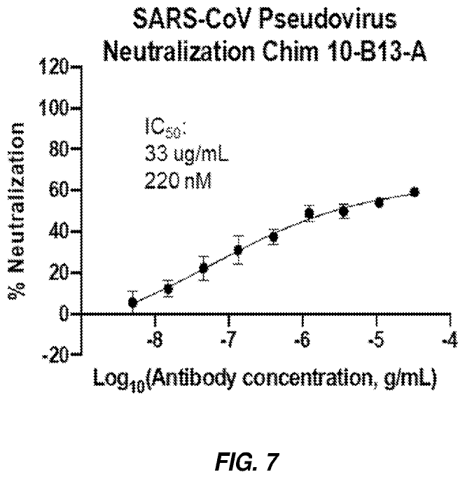

FIG. 7 shows IC50 titration of 10-B13-A (human Fc IgG2 chimera) in SARS-CoV-1 pseudovirus ACE2+TMPRSS2+ target cell infection assay. IC50 values were determined by fitting the dose-response curves with four-parameter logistic regression in Prism GraphPad (version 8.1.2). All data was normalized to pseudovirus alone. All error bars represent S.D. from three replicates. The IC50 value (33 ug/ml, 220 nM) was estimated based on the data.

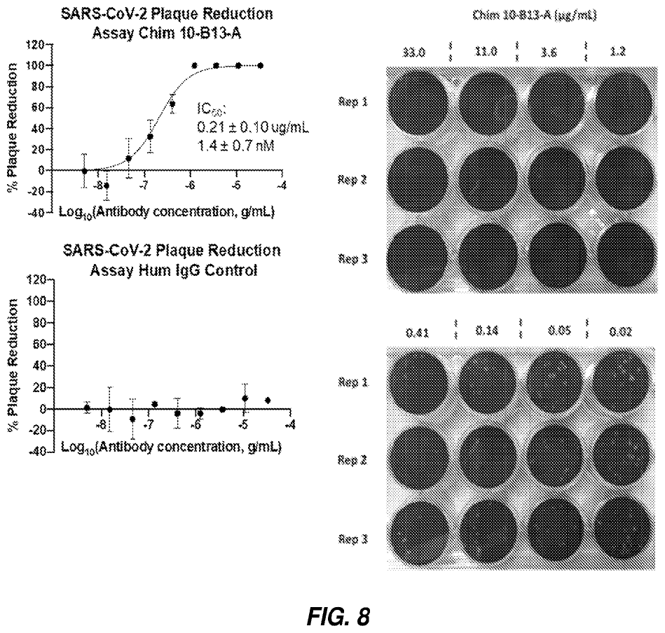

FIG. 8 shows IC50 titration of 10-B13-A (human Fc IgG2 chimera) in BSL3 Vero E6 infection plaque assay. Human IgG was included as a negative control. The IC50 value (0.21±0.10 ug/mL, 1.4±0.7 nM) was determined by fitting the dose-response curves with four-parameter logistic regression in Prism GraphPad (version 8.1.2). All data was normalized to virus alone. All error bars represent S.D. from three replicates.

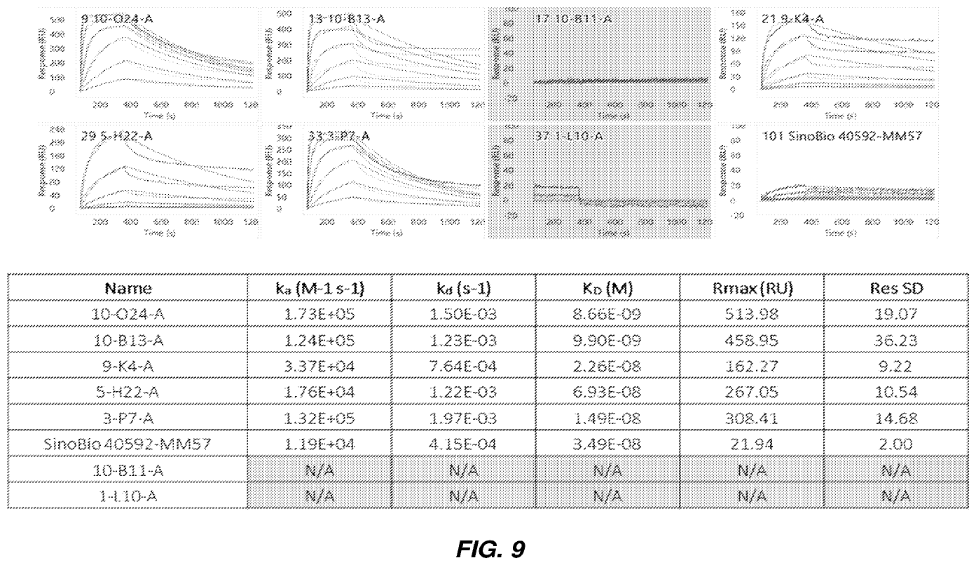

FIG. 9 shows binding kinetics for selected SARS-CoV-2 spike-binding mAbs against RBD. Carterra LSA was used to determine on/off rates and binding affinities (KD). Candidate antibodies (ligands) were coupled to a HC30M chip and blocked. Serial dilutions (1000 nM start, 1:3 dilution, 8 points) of RBD were injected for kinetic constant determination. At the end of each cycle, the chip was regenerated to remove bound antigen. Kinetics analysis was performed using Carterra kinetics software. 10-B11-A and 1-L10-A do not bind the RBD; spike-binding mAb SinoBio 40592-MM57 was included as a positive control.

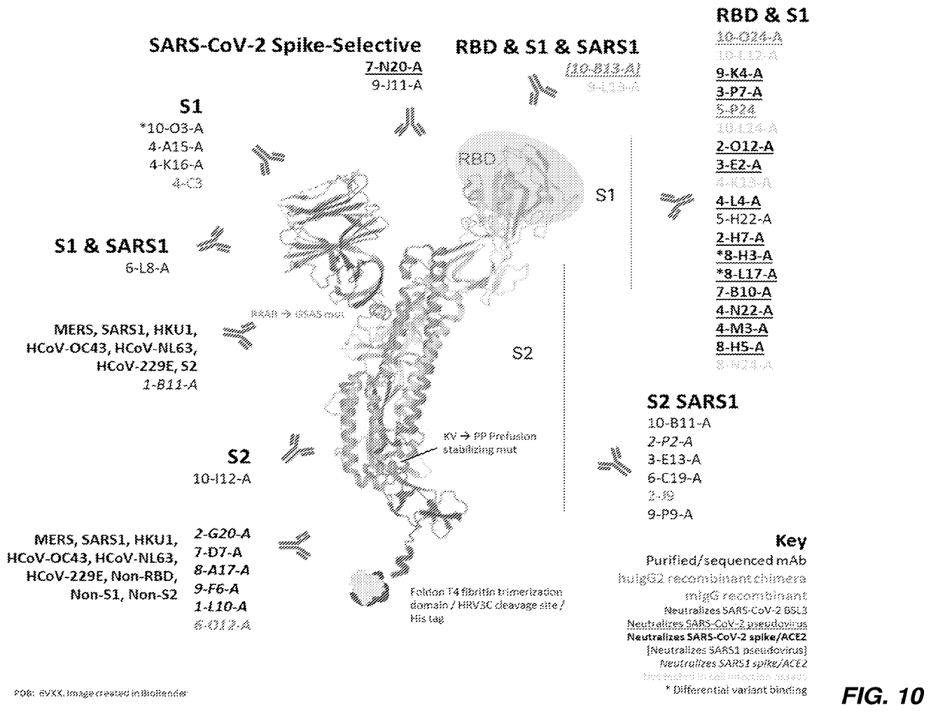

FIG. 10 provides a binding and functional summary of 42 SARS-CoV-2 spike binding mAbs. A wide variety of antibodies with a range of binding and functional activities are summarized. Certain antibodies are specific for SARS-CoV-2 RBD, cross-react with SARS-CoV-1, bind to three CDC variants of concern, and neutralize both SARS-CoV-2 and SARS-CoV-1 in in vitro infection models (e.g. 10-B13-A) even when reformatted as a human chimera (IgG2 Fc). Certain antibodies can be produced recombinantly with a mouse IgG2b Fc and are specific to non-RBD domains of the S1 domain (e.g. 4-C3-A). Certain antibodies are specific for the S2 domain of SARS-CoV2 (e.g. 10-I12-A); some also cross-react with SARS-CoV-1 (e.g. 10-B11-A); some also cross-react with all of the coronavirus spike proteins known to infect humans (e.g. 1-B11-A). Certain neutralizing antibodies are selective for SARS-CoV-2 spike trimer and do not seem to bind to recombinantly-expressed subdomains (e.g 7-N20-A). All antibodies bind to B.1.1.7, B.1.351, and P.1 spike variants of concern except 10-O3-A, which only binds to P.1; and 8-H3-A and 8-L17-A, which only bind to B.1.1.7.

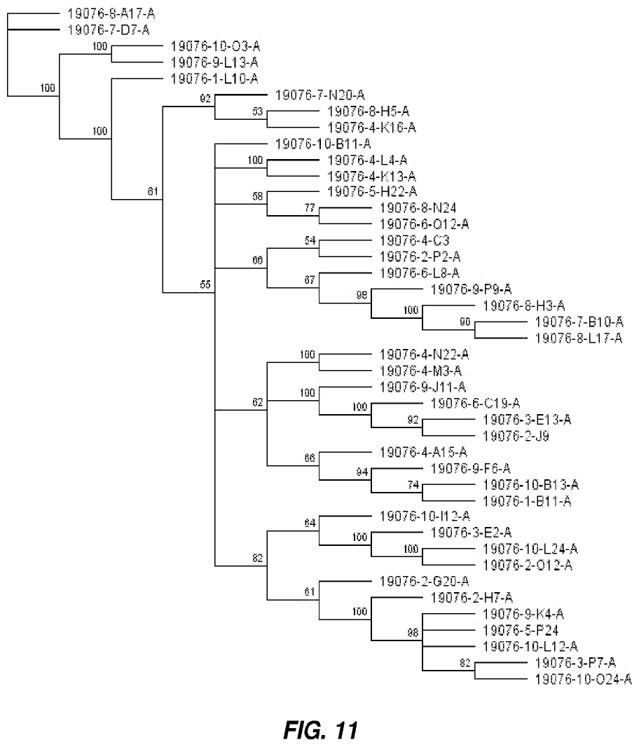

FIG. 11 shows a SARS-CoV-2 spike binding mAb dendrogram. A phylogenetic alignment for 42 mAb amino acid sequences was built by MUSCLE alignment and Neighbor-joining using Geneious software. The heavy chain and light chain sequences for each mAb were concatenated into one sequence (separated by a 4×GGGS linker). The confidence (%) after resampling against the consensus tree is displayed at each node.

### Example 2: Mouse Antibodies with Activities Against the SARS-CoV-2 D614G and B.1.351 Variants

With the rapid spread of SARS-CoV-2 variants, including those that are resistant to antibodies authorized for emergency use, it is becoming apparent that new antibodies may be needed to effectively protect patients against more severe disease. The difference between the murine and human antibody repertoire may allow for the isolation of murine monoclonal antibodies that recognize a different or broader range of SARS-CoV-2 variants than the human antibodies that have been characterized so far. As demonstrated herein, mouse antibodies B13 and O24 demonstrate neutralizing potency against SARS-CoV-2 D614G and B.1.351 variants. Such murine antibodies may have an advantage in protecting against severe symptoms when individuals are exposed to new SARS-CoV-2 variants.

As the COVID-19 pandemic progresses, various resistant strains have begun to spread through the population, and it has been recognized that new antibodies will need to be added to the armamentarium in order to continue to effectively provide prophylactic and/or therapeutic value to patients. Even as more monoclonal therapeutic antibodies, identified in patients infected with wild-type acute respiratory syndrome coronavirus 2 (SARS-CoV-2), are authorized for emergency use, the number of SARS-CoV-2 variants that harbor mutations in the viral spike protein are increasing in incidence throughout the world (Ozono et al., “SARS-CoV-2 D614G Spike Mutation Increases Entry Efficiency with Enhanced Ace2-Binding Affinity,” Nat Commun 12:848 (2021); Hoffman et al., “SARS-CoV-2 Variants B.1.351 and P.1 Escape from Neutralizing Antibodies,” Cell 184(9):2384-2393.e12 (2021), each of which is hereby incorporated by reference in its entirety), raising questions on the long-term efficacy of current vaccines and therapeutic antibodies.

Some of the new variants of SARS-CoV-2 have been termed variants of concern (VOC) by the World Health Organization (WHO) and the Centers for Disease Control and Prevention (CDC) (Davies et al., “Estimated Transmissibility and Impact of SARS-CoV-2 Lineage B.1.1.7 in England,” MedRXiv doi:10.1101/2020.12.24.20248822 (2021); Tegally et al., “Emergence and Rapid Spread of a New Severe Acute Respiratory Syndrome-related Coronavirus 2 (SARS-CoV-2) Lineage with Multiple Spike Mutations in South Africa,” MedRXiv doi:10.1101/2020.12.21.20248640 (2020); Deng et al., “Transmission, Infectivity, and Antibody Neutralization of an Emerging SARS-CoV-2 Variant in California Carrying a L452R Spike Protein Mutation,” MedRxiv doi:10.1101/2021.03.07.21252647 (2021), each of which is hereby incorporated by reference in its entirety) because they show increased transmissibility, increased virulence, or decreased effectiveness of vaccines and therapeutics (see WHO website at www.who.int/en/activities/tracking-SARS-CoV-2-variants/). These include the Alpha (B.1.1.7), Beta (B.1.351), Gamma (P.1), Delta (B.1.617.2) and Epsilon (B.1.427/B.1.429) SARS-CoV-2 variants (formerly, the United Kingdom, South Africa, Brazil, India, and California variants, respectively) (Chand et al., Investigation of Novel SARS-COV-2 Variant: Variant of Concern 202012/01 (Report); Davies et al., “Estimated Transmissibility and Impact of SARS-CoV-2 Lineage B.1.1.7 in England,” MedRXiv doi:10.1101/2020.12.24.20248822 (2021); Tegally et al., “Emergence and Rapid Spread of a New Severe Acute Respiratory Syndrome-related Coronavirus 2 (SARS-CoV-2) Lineage with Multiple Spike Mutations in South Africa,” MedRXiv doi:10.1101/2020.12.21.20248640 (2020), each of which is hereby incorporated by reference in its entirety). In addition to increased transmissibility, the B.1.351, P.1 and B.1.427/B.1.429 variants have demonstrated reduced susceptibility to a combination of two therapeutic monoclonal antibodies, bamlanivimab (LY-CoV555) and etesevimab (LY-CoV016) (Hoffman et al., “SARS-CoV-2 Variants B.1.351 and P.1 Escape from Neutralizing Antibodies,” Cell 184(9):2384-2393.e12 (2021); Liu et al., “Potent Neutralizing Antibodies Against Multiple Epitopes on SARS-CoV-2 Spike,” Nature 584:450-456 (2020); Pearson et al., “Estimates of Severity and Transmissibility of Novel South Africa SARS-CoV-2 Variant 501Y.V2,” retrieved from cmmid.github.io/, each of which is hereby incorporated by reference in its entirety).

Antibodies isolated from convalescent sera that neutralize the original SARS-CoV-2 isolates recognize a variety of distinct non-overlapping epitopes in the receptor-binding domain (RBD) of the spike protein (Barnes et al., “SARS-CoV-2 Neutralizing Antibody Structures Inform Therapeutic Strategies,” Nature 588:682-687 (2020); Brouwer et al., “Potent Neutralizing Antibodies from COVID-19 Patients Define Multiple Targets of Vulnerability,” Science 369:643-650 (2020); Ju et al., “Human Neutralizing Antibodies Elicited by SARS-CoV-2 Infection,” Nature 584:115-119 (2020); Liu et al., “501Y.V2 and 501Y.V3 Variants of SARS-CoV-2 Lose Binding to Bamlanivimab in vitro,” bioRxiv doi:10.1101/2021.02.16.431305 (2021), each of which is hereby incorporated by reference in its entirety). These antibodies, as well as antibodies from immunized individuals, frequently do not recognize at least a subset of the new strains of virus. Selection and spread of mutated disease vectors may be driven by better fitness, such as increased transmission. However, although some variants show increased transmission, as described above, many mutants do not show an improvement in fitness. It is possible that these variants are able to spread because they are not recognized by the prevalent human responses to the wild-type viruses or they have arisen by selection against the human repertoire. The difference in the murine and human antibody repertoire may allow the isolation of murine monoclonal antibodies that recognize a different or broader range of SARS-CoV-2 variants than the human antibodies that have been characterized so far.

This Example describes the activities of two mouse monoclonal antibodies, B13 and O24, obtained from mice using the PentaMice™ platform, that recognize RBD in neutralization assays against wild-type SARS-CoV-2 virus and SARS-CoV-2 pseudovirus variants. Both antibodies demonstrate excellent neutralizing potency against wild-type SARS-CoV-2 and other variants tested. B13 also binds to SARS-CoV-1. These antibodies, with their broad specificity against new variants of SARS-CoV-2 virus, provide promising candidates for therapy.

### Materials and Methods

Cells and viruses: Vero E6 cells (ATCC) were cultured in Dulbecco's modified Eagle's medium (DMEM) supplemented with 10% (v/v) fetal bovine serum (FBS) and 2 mM penicillin-streptomycin (100 U/mL). 293T-hsACE2 cells (Cat #C-HA102) were purchased from Integral Molecular and cultured according to manufacturer's recommendations. Pseudotyped Wuhan D614G (Cat #RVP-702L); and B.1.351 (Cat #RVP-707L) were purchased from Integral Molecular, Philadelphia, PA.

Antibodies: LY-CoV555 (bamlanivimab), LY-CoV016 (etesevimab), AZD1061 (cilgavimab), AZD8895 (tixagevimab), VIR-7831 (sotrovimab), CT-P59 (regdanvimab), REGN10987 (imdevimab), and REGN10933 (casirivimab) were expressed expressed in Chinese hamster ovary (CHO) cells and purified by Protein A affinity chromatography. Production of proteins was carried out by transient expression in CHO-K1 cells adapted to serum-free suspension culture (TunaCHO™, LakePharma Inc., Belmont, CA). Constructs were introduced into the LakePharma proprietary expression vector. Suspension CHO cells were seeded in a shake flask and expanded using a serum-free and chemically defined medium. On the day of transfection, the expanded cells were seeded into a new vessel with fresh medium. Transient transfections were done with the addition of the DNA and transfection reagents, under high density conditions as previously described. Transfections were carried out in cultures of 0.1 to 2.0 liters. After transfection, the cells were maintained as a batch-fed culture in a shake flask until the end of the production run. The conditioned cell culture fluid was harvested after 7-14 days, clarified by centrifugation and sterile-filtered, prior to purification. The culture supernatant was applied to a column packed with CaptivA© Protein A Affinity Resin (Repligen, Massachusetts, USA) pre-equilibrated with 137 mM NaCl, 2.7 mM KCl, 10 mM Na₂HPO₄, 2 mM KH₂PO₄ pH 7.4 (PBS). The column was washed with the PBS buffer until the OD280 value returned to baseline. The target protein was then eluted with 0.25% acetic acid buffer at pH 3.5. Fractions were collected, buffered with 1 M HEPES, and the OD280 value of each fraction was recorded. Fractions containing the target protein were pooled, formulated into 100 mM HEPES, 100 mM NaCl, 50 mM NaOAc, pH 6.0, and filtered through a 0.2 μm membrane filter. and stored at 4□ prior to use. The protein concentration was calculated from the OD280 value and the calculated extinction coefficient. B13 and O24 are monoclonal antibodies isolated from the PentaMice™ platform (LakePharma Inc., Belmont, CA) after immunization with SARS-CoV-2 spike trimer protein. The B13 and O24 mouse variable (V) regions were expressed as human chimeric antibodies combining the human immunoglobulin G1 (IgG1) and kappa chain constant regions (FIG. 16).

Pseudovirus SARS-CoV-2 neutralization assay: The neutralization assay was carried out according to the manufacturers' protocols. In brief, serially diluted antibodies were incubated with pseudotyped SARS-CoV-2-Luciferase for 1 hr at 37° C. At least nine concentrations were tested for each antibody. Pseudovirus in culture media without antibody was used as a negative control to determine 100% infectivity. The mixtures were then incubated with 293T-hsACE2 cells at 2.5×10e5 cells/ml in the 96-well plates. Infection took place over approximately 72 hrs at 37° C. with 5% CO₂. The luciferase signal was measured using the Renilla-Glo luciferase assay system (Promega, Cat #E2710) with the luminometer set at 1 ms integration time. The obtained relative luminescence signals (RLU) from the negative control wells were normalized and used to calculate the neutralization percentage for each concentration. These data were processed by Prism 9 (GraphPad) to fit a 4PL curve and calculate the log IC₅₀.

ELISA: Cross-reactivity of B13 and O24 against SARS-CoV-1 spike protein was determined by ELISA. In brief, SARS-CoV-1 spike protein (Uniprot seq: P59594) containing an engineered carboxyl-terminal T4 fibritin trimerization domain was expressed using the TunaCHO™ platform (LakePharma) and used to coat wells in a 384-well plate (1 μg/mL in PBS) overnight at 4° C. The wells were then washed twice (PBS with 0.05% Tween-20) and blocked (PBS with 3% BSA) for 1 h at room temperature (RT). The blocking solution was discarded, and serially diluted antibodies (3-fold dilutions from 0.001-200 nM) were added to the wells and incubated 1 hour at RT. The plates were then washed 4 times, and then goat anti-mouse IgG-HRP (Jackson ImmunoResearch, 1:7,000 dilution in PBS with 3% BSA) was added to the wells and incubated for 1 hour at RT. The plates were then washed 8 times, and chemiluminescent substrate was added (SuperSignal ELISA Pico substrate solution, Thermo, per manufacturer's instructions). Within 15 minutes of adding substrate, the plates were read on a Molecular Devices SpectraMax M3 luminometer with Softmax Pro Version 6.2. These data were processed by Prism 9 (GraphPad) to fit a 4PL curve and calculate the log EC₅₀.

### Results

B13 demonstrates excellent neutralizing potency against SARS-CoV-2: To evaluate whether B13 can neutralize wild-type SARS-CoV-2 in vitro, a live virus assay was performed. Vero E6 cells were cocultured with live virus and monoclonal antibody for 20 hours before measuring fluorescence. B13 inhibited infection of this virus with an IC₅₀ value of 19 pM (FIG. 14).

O24, unlike B13, shows equivalent neutralization activity against SARS-CoV-2 D614G and B.1.351: To assess the neutralizing efficacy of a panel of antibodies against SARS-CoV-2 D614G and B.1.351 variant, a pseudovirus-based in vitro assay was utilized. 293T-hsACE2 cells were cocultured with reporter virus particles in the presence or absence of the antibodies for 72 hours before luminescence was measured. B13 effectively neutralized SARS-CoV-2 D614G with an IC₅₀ value of 52 pM (FIGS. 13 and 14) but showed a reduced potency of 1.53 nM against the B.1.351. 024's activity against D614G was comparable to B13, with an IC₅₀ value of 24 pM. However, O24 at 12 pM, had a 128-fold improvement in potency against the B.1.351 variant compared to B13 (FIG. 14).

B13 but not O24 binds to SARS-CoV-1 spike: To ask if B13 and/or O24 has the potential for pan-coronavirus activity, the mAbs were tested for binding to the SARS-CoV-1 spike protein, which shares 76% identity (73% identity in the RBD domain) with SARS-CoV-2. B13 but not O24 was a potent SARS-CoV-1 spike binder, with an ELISA EC50 of approximately 1 nM (FIG. 15).

### Discussion

Neutralizing monoclonal antibodies targeting the SARS-CoV-2 spike protein have tremendous therapeutic potential by mitigating the symptoms of patients with mild to moderate COVID-19 or preventing infection altogether (Jiang et al., “Neutralizing Antibodies Against SARS-CoV-2 and Other Human Coronaviruses,” Trends Immunol 41:355-359 (2010), which is hereby incorporated by reference in its entirety). Several of these antibodies have been authorized for emergency therapeutic use as mono- or cocktail therapies, including REGN10987 (imdevimab), REGN10933 (casirivimab), LY-CoV555 (bamlanivimab), LY-CoV016 (etesevimab), and VIR-7831 (sotrovimab). Others, including AZD1061 (cilgavimab), AZD8895 (texagevimab), and CT-P59 (regdanivimab), are currently awaiting authorization. Recently, however, due to the increase of the incidence of variants, emergency use authorization of bamlanivimab as a monotherapy was revoked (see “Coronavirus (COVID-19) Update: FDA Revokes Emergency Use Authorization for Monoclonal Antibody Bamlanivimab,” U.S. Food and Drug Administration (FDA) (Press release)(2021); Fact Sheet For Health Care Providers Emergency Use Authorization (Eua) Of Bamlanivimab and Etesevimab, 02092021) and its use in combination with etesevimab has been paused in certain states (see “Fact Sheet For Health Care Providers Emergency Use Authorization (Eua) Of Regen-Cov (fda.gov); www.phe.gov/emergency/events/COVID19/investigation-MCM/Bamlanivimab-etesevimab/Pages/default.aspx, each of which is hereby incorporated by reference in its entirety), prompting further concern about the long-term efficacy of neutralizing antibodies in development as variants continue to emerge.

Concerns have also been raised as new SARS-CoV-2 variants are emerging worldwide (Tada et al., “Decreased Neutralization of SARS-CoV-2 Global Variants by Therapeutic Anti-spike Protein Monoclonal Antibodies,” bioRxiv doi:10.1101/2021.02. 18.431897; Hoffman et al., “SARS-CoV-2 Variants B.1.351 and P.1 Escape from Neutralizing Antibodies,” Cell 184(9):2384-2393.e12 (2021); Liu et al., “Potent Neutralizing Antibodies Against Multiple Epitopes on SARS-CoV-2 Spike,” Nature 584:450-456 (2020); Pearson et al., “Estimates of Severity and Transmissibility of Novel South Africa SARS-CoV-2 Variant 501Y.V2,” retrieved from cmmid.github.io/, each of which is hereby incorporated by reference in its entirety). These new variants, especially the B.1.351 variant, clearly demonstrate antigenic drift, showing resistance to neutralizing antibodies typically derived from convalescent serum, resulting in the increase of transmissibility of SARS-CoV-2 (Wang et al., “Antibody Resistance of SARS-CoV-2 Variants B.1.351 and B.1.1.7,” Nature 593:130-5 (2021), which is hereby incorporated by reference in its entirety). These concerns regarding resistance to neutralizing antibodies are supported by our data demonstrating the resistance of the B.1.351 variant to neutralization by LY-CoV555, LY-CoV016, and REGN10933. Interestingly, the most potent antibody against D614G, CT-P59, has reduced potency against the B.1.351 variant.

In order to avoid resistance to antibody neutralization by new variants, antibody recognition was expanded beyond the limited antigenic sites on RBD recognized by current human antibodies. The mouse immunoglobulin repertoire was therefore utilized (Collins and Jackson, “On Being the Right Size: Antibody Repertoire Formation in the Mouse and Human,” Immunogenetics 70:143-158 (2018), which is hereby incorporated by reference in its entirety) through hybridoma technology to isolate mouse monoclonal antibodies. Two antibodies, B13 and O24, were selected and identified as potent neutralizers of wild-type SARS-CoV-2. B13 was also cross-reactive to SARS-CoV-1 (FIG. 15) but demonstrated a slight decrease in potency against the B.1.351 variant. In contrast, O24 also effectively neutralized the B.1.351 variant. These findings indicate that the selection of mouse antibodies such as B13 and O24, unlike antibodies derived from convalescent patient plasma, may provide substantial additional coverage to that afforded by human-derived monoclonal antibodies against the induction of severe symptoms when individuals are exposed to new SARS-CoV-2 variants.

The examples set forth above are provided to give those of ordinary skill in the art a complete disclosure and description of how to make and use the embodiments of the compositions, systems and methods of the invention, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of those skilled in the art to which the invention pertains. All references cited in this disclosure are incorporated by reference to the same extent as if each reference had been incorporated by reference in its entirety individually.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

What is claimed is:

1. A monoclonal antibody, or antigen binding portion thereof, that binds to SARS-Cov-2 spike protein (CoV-S), wherein the antibody comprises the vhCDR1, vhCDR2, vhCDR3, vlCDR1, vlCDR2 and vlCDR3 from an antibody selected from the group consisting of clone IDs: 3-P7-A (FIG. 12H), 5-P24-A (FIG. 12N), 9-K4-A (FIG. 12R), 10-B13-A (FIG. 12V), 10-L12-A (FIG. 12W), and 10-O24-A (FIG. 12Y).

2. The monoclonal antibody, or antigen binding portion thereof, according to claim 1, which comprises:
(i) the vhCDR1, vhCDR2, vhCDR3, vlCDR1, vlCDR2 and vlCDR3 sequences of SEQ ID NOS:76-81;
(ii) the vhCDR1, vhCDR2, vhCDR3, vlCDR1, vlCDR2 and vlCDR3 sequences of SEQ ID NOS:136-141;
(iii) the vhCDR1, vhCDR2, vhCDR3, vlCDR1, vlCDR2 and vlCDR3 sequences of SEQ ID NOS:176-181;
(iv) the vhCDR1, vhCDR2, vhCDR3, vlCDR1, vlCDR2 and vlCDR3 sequences of SEQ ID NOS:216-221;
(v) the vhCDR1, vhCDR2, vhCDR3, vlCDR1, vlCDR2 and vlCDR3 sequences of SEQ ID NOS:226-231; or
(vi) the vhCDR1, vhCDR2, vhCDR3, vlCDR1, vlCDR2 and vlCDR3 sequences of SEQ ID NOS:246-251.

3. The monoclonal antibody, or antigen binding portion thereof, according to claim 1, which comprises:
(i) the variable heavy domain (VH) and variable light domain (VL) according to SEQ ID NOS: 72 and 74;
(ii) the VH and VL according to SEQ ID NOS: 132 and 134;
(iii) the VH and VL according to SEQ ID NOS: 172 and 174;
(iv) the VH and VL according to SEQ ID NOS: 212 and 214;
(v) the VH and VL according to SEQ ID NOS: 222 and 224; or
(vi) the VH and VL according to SEQ ID NOS: 242 and 244.

4. The monoclonal antibody according to any one of claims 1 to 3, wherein the monoclonal antibody is humanized.

5. The monoclonal antibody according to any one of claims 1 to 3, wherein the monoclonal antibody is IgG1, IgG2, IgG3, or IgG4 class.

6. The antigen binding portion of the monoclonal antibody according to any one of claims 1 to 5.

7. The antigen binding portion according to claim 6, wherein the antigen binding portion comprises a Fab fragment, Fv fragment, or single-chain Fv antibody.

8. A composition comprising a monoclonal antibody, or antigen binding portion thereof, according to any one of claims 1 to 7.

9. The composition according to claim 8, wherein the monoclonal antibody, or antigen binding portion thereof, comprises the vhCDR1, vhCDR2, vhCDR3, vlCDR1, vlCDR2 and vlCDR3 sequences of: (i) SEQ ID NOS:76-81, (ii) SEQ ID NOS:136-141, (iii) SEQ ID NOS:176-181, (iv) SEQ ID NOS:216-221, (v) SEQ ID NOS:226-231, or (vi) SEQ ID NOS:246-251.

10. The composition according to claim 8, wherein the monoclonal antibody, or antigen binding portion thereof, comprises (i) the VH and VL domains according to SEQ ID NOS: 72 and 74, (ii) the VH and VL domains according to SEQ ID NOS: 132 and 134; (iii) the VH and VL domains according to SEQ ID NOS: 172 and 174; (iv) the VH and VL domains according to SEQ ID NOS: 212 and 214; (v) the VH and VL domains according to SEQ ID NOS: 222 and 224; or (vi) the VH and VL domains according to SEQ ID NOS: 242 and 244.

11. A cell line that expresses the monoclonal antibody, or antigen binding portion thereof, according to any one of claims 1 to 7.

12. A composition comprising:
a first nucleic acid encoding the variable heavy domain (VH) of the antibody of any one of claims 1 to 7, and
a second nucleic acid encoding the variable light domain (VL) of the same antibody.

13. The composition according to claim 12, wherein the first nucleic acid molecule encodes the vhCDR1, vhCDR2, and vhCDR3 sequences of: (i) SEQ ID NOS:76-78, (ii) SEQ ID NOS:136-138, (iii) SEQ ID NOS:176-178, (iv) SEQ ID NOS:216-218, (v) SEQ ID NOS:226-228, or (vi) SEQ ID NOS:246-248.

14. The composition according to claim 12, wherein the second nucleic acid molecule encodes the vlCDR1, vlCDR2 and vlCDR3 sequences of: (i) SEQ ID NOS:79-81, (ii) SEQ ID NOS:139-141, (iii) SEQ ID NOS:179-181, (iv) SEQ ID NOS:219-221, (v) SEQ ID NOS:229-231, or (vi) SEQ ID NOS:249-251.

15. The composition according to claim 12, wherein the first nucleic acid molecule encodes the VH domain comprising the amino acid sequence of one of SEQ ID NOS: 72, 132, 172, 212, 222, and 242.

16. The composition according to claim 12, wherein the second nucleic acid molecule encodes the VL domain comprising the amino acid sequence of one of SEQ ID NOS: 74, 134, 174, 214, 224, and 244.

17. An expression vector comprising the first and second nucleic acids of any one of claims 12 to 16.

18. A host cell comprising the expression vector of claim 17.

19. A method of making an antibody, or antigen binding portion thereof, comprising:
culturing the host cell of claim 18 under conditions wherein the antibody, or antigen binding portion thereof, is produced; and
recovering the antibody or antigen binding portion thereof.

20. A method of treating or preventing SARS-CoV-2 infection in a patient in need comprising administering to the patient the monoclonal antibody, or antigen binding portion thereof, according to any one of claims 1 to 7.

21. A method of detecting SARS-CoV-2 in a human sample comprising:
contacting the human sample with the monoclonal antibody, or antigen binding portion thereof, according to any one of claims 1 to 7, and
detecting binding of the antibody, or antigen binding portions thereof, to SARS-CoV-2 spike protein (CoV-S) as an indication of presence of SARS-CoV-2 in the sample.


SEQUENCE LISTING (425 sequences)

SEQ ID NO: 1 - protein, 1262 aa, Artificial
  SARS-CoV-2 Spike Protein (R691G, R692S, R694A, K995P, V996P)-T4 fibritin-HRV3C-His8 Fusion Polypeptide
MEWSWVFLFFLSVTTGVHSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGYIPEAPRDGQAYVRKDGEWVLLSTFLLEVLFQGPHHHHHHHH

SEQ ID NO: 2 - protein, 119 aa, Artificial
  Heavy chain variable domain of antibody 1-B11-A
QVQLQQSGPELVKPGASVKISCKASGYTFTDHYINWVKQRPGQGLEWIGWIFPGSGSTYYNEQFKGKATLTVDKSSNTAYMLLSSLTSEDSAVYFCARSGGLRLGFAYWGQGTLVTVSA

SEQ ID NO: 3 - DNA, 357 bp, Artificial
  Heavy chain variable domain of antibody 1-B11-A
caggtccagctacagcagtctggacctgaactggtgaagcctggggcttcagtgaagatatcctgcaaggcttctggctacaccttcactgaccactatataaattgggtgaagcagaggcctggacagggacttgagtggattggatggatttttcctggaagtggtagtacttactacaatgagcagttcaagggcaaggccacgcttactgtagacaaatcctccaacacagcctacatgttgctcagcagcctgacctctgaggactctgcggtctatttctgtgcaagatcagggggattacgactggggtttgcttactggggccaagggactctggtcactgtctctgca

SEQ ID NO: 4 - protein, 107 aa, Artificial
  Light chain variable domain of antibody 1-B11-A
DIQMTQTTSSLSASLGDRVTISCRASQDISNYLNWYQQKPDGTVKLLIYYTSRLHSGVPSRFSGSGSGTDYSLTISNLEQEDISTYFCQQVNTLPYTFGGGTKLEIK

SEQ ID NO: 5 - DNA, 321 bp, Artificial
  Light chain variable domain of antibody 1-B11-A
gatatccagatgacacagactacatcctccctgtctgcctctctgggagacagagtcaccatcagttgcagggcaagtcaggacattagcaattatttaaactggtatcagcagaaaccagatggaactgttaaactcctgatctactacacatcaagattacactcaggagtcccatcaaggttcagtggcagtgggtctggaacagattattctctcaccattagcaacctggagcaagaagatatttccacttacttttgccaacaggttaatacgcttccgtacacgttcggaggggggaccaagctggaaataaaa

SEQ ID NO: 6 - protein, 8 aa, Artificial
  HC-CDR1 of antibody 1-B11-A
GYTFTDHY

SEQ ID NO: 7 - protein, 8 aa, Artificial
  HC-CDR2 of antibody 1-B11-A
IFPGSGST

SEQ ID NO: 8 - protein, 12 aa, Artificial
  HC-CDR3 of antibody 1-B11-A
ARSGGLRLGFAY

SEQ ID NO: 9 - protein, 6 aa, Artificial
  LC-CDR1 of antibody 1-B11-A
QDISNY

SEQ ID NO: 10 - protein, 3 aa, Artificial
  LC-CDR2 of antibody 1-B11-A
YTS

SEQ ID NO: 11 - protein, 9 aa, Artificial
  LC-CDR3 of antibody 1-B11-A
QQVNTLPYT

SEQ ID NO: 12 - protein, 116 aa, Artificial
  Heavy chain variable domain of antibody 1-L10-A
EVKLEESGGGLVQPGGSMKLSCVASGFTFNNYWMNWVRQSPEKGLEWVAQMRLKSDNYATHYAESVKGRFTISRDDSKSSVYLQMNNLRAEDTGIYYCIEGYWTYWGQGTLVTVSA

SEQ ID NO: 13 - DNA, 348 bp, Artificial
  Heavy chain variable domain of antibody 1-L10-A
gaagtgaagcttgaggagtctggaggaggcttggtgcaacctggaggatccatgaaactctcctgtgttgcctctggattcactttcaataactactggatgaactgggtccgccagtctccagagaaggggcttgagtgggttgctcaaatgagattgaaatctgataattatgcaacacattatgcggagtctgtgaaagggaggttcaccatctcaagagatgattccaaaagtagtgtctatctgcaaatgaacaacttaagggctgaagacactggaatttattactgtattgaaggttactggacttactggggccaagggactctggtcactgtctctgca

SEQ ID NO: 14 - protein, 109 aa, Artificial
  Light chain variable domain of antibody 1-L10-A
QIVLTQSPAIMSASLGERVTMTCTASSSVSSSYLHWYQQKPGSSPKLWIYSTSNLASGVPARFSGNGSGTSYSLTISSMEAEDAATYYCLQYHRSPPYTFGGGTKLEIK

SEQ ID NO: 15 - DNA, 327 bp, Artificial
  Light chain variable domain of antibody 1-L10-A
caaattgttctcacccagtctccagcaatcatgtctgcatctctaggggaacgggtcaccatgacctgcactgccagctcaagtgtaagttccagttacttgcactggtaccagcagaagccaggatcctcccccaaactctggatttatagcacatccaacctggcttctggagtcccagctcgcttcagtggcaatgggtctgggacctcttactctctcacaatcagcagcatggaggctgaagatgctgccacttattactgcctccagtatcatcgctccccaccgtacacgttcggaggggggaccaagctggaaataaaa

SEQ ID NO: 16 - protein, 8 aa, Artificial
  HC-CDR1 of antibody 1-L10-A
GFTFNNYW

SEQ ID NO: 17 - protein, 10 aa, Artificial
  HC-CDR2 of antibody 1-L10-A
MRLKSDNYAT

SEQ ID NO: 18 - protein, 7 aa, Artificial
  HC-CDR3 of antibody 1-L10-A
IEGYWTY

SEQ ID NO: 19 - protein, 7 aa, Artificial
  LC-CDR1 of antibody 1-L10-A
SSVSSSY

SEQ ID NO: 20 - protein, 3 aa, Artificial
  LC-CDR2 of antibody 1-L10-A
STS

SEQ ID NO: 21 - protein, 10 aa, Artificial
  LC-CDR3 of antibody 1-L10-A
LQYHRSPPYT

SEQ ID NO: 22 - protein, 120 aa, Artificial
  Heavy chain variable domain of antibody 2-H7-A
QAYLQQSGAELVRPGASVKMSCKASGYTFTGYTMHWVKQTPRQGLEWIGAIYPENGDTSYNQKFKGKATLTVDKSSSTAYMQLSSLTSEDSAVYFCARDGYDVLYGMDYWGQGTSVTVSS

SEQ ID NO: 23 - DNA, 360 bp, Artificial
  Heavy chain variable domain of antibody 2-H7-A
caggcttatctacagcagtctggggctgagctggtgaggcctggggcctcagtgaagatgtcctgcaaggcttctggctacacatttaccggttacactatgcactgggtaaagcagacacctagacagggcctggaatggattggagctatttatccagaaaatggtgatacttcctacaatcagaagttcaagggcaaggccacactgactgtcgacaaatcctccagcacagcctacatgcagctcagcagcctgacatctgaagactctgcggtctatttctgtgcaagagatggttacgacgttctctatggtatggactactggggtcaaggaacctcagtcaccgtctcctca

SEQ ID NO: 24 - protein, 111 aa, Artificial
  Light chain variable domain of antibody 2-H7-A
DIVLTQSPASLAVSLGQRATISCRASKSVSTSGYSYMHWYQQKPGQPPKLLIYLASNLESGVPARFSGSGSGTDFTLNIHPVEEEDAATYYCQHSRELPWTFGGGTKLDIK

SEQ ID NO: 25 - DNA, 333 bp, Artificial
  Light chain variable domain of antibody 2-H7-A
gacattgtgctgacacagtctcctgcttccttagctgtatctctggggcagagggccaccatctcatgcagggccagcaaaagtgtcagtacatctggctatagttatatgcactggtaccaacagaaaccaggacagccacccaaactcctcatctatcttgcatccaacctagaatctggggtccctgccaggttcagtggcagtgggtctgggacagacttcaccctcaacatccatcctgtggaggaggaggatgctgcaacctattactgtcagcacagtagggagcttccgtggacgttcggtggaggcaccaagctggatatcaaa

SEQ ID NO: 26 - protein, 8 aa, Artificial
  HC-CDR1 of antibody 2-H7-A
GYTFTGYT

SEQ ID NO: 27 - protein, 8 aa, Artificial
  HC-CDR2 of antibody 2-H7-A
IYPENGDT

SEQ ID NO: 28 - protein, 13 aa, Artificial
  HC-CDR3 of antibody 2-H7-A
ARDGYDVLYGMDY

SEQ ID NO: 29 - protein, 10 aa, Artificial
  LC-CDR1 of antibody 2-H7-A
KSVSTSGYSY

SEQ ID NO: 30 - protein, 3 aa, Artificial
  LC-CDR2 of antibody 2-H7-A
LAS

SEQ ID NO: 31 - protein, 9 aa, Artificial
  LC-CDR3 of antibody 2-H7-A
QHSRELPWT

SEQ ID NO: 32 - protein, 124 aa, Artificial
  Heavy chain variable domain of antibody 2-J9-A
EVQLQQSGPELVKPGASVKMSCKASGYTFTDYNIHWVKQSHGKSLEWIGYINPNNGGTDYNQKFQGKATLTVNKSSSTAYMELRSLTSEDSAVYYCARGGFYGNYGAYYGMDFWGQGTSVTVSS

SEQ ID NO: 33 - DNA, 372 bp, Artificial
  Heavy chain variable domain of antibody 2-J9-A
gaggtccagctgcaacagtctggacctgagttggtgaagcctggggcttcagtgaagatgtcctgcaaggcttctggatacacattcactgactacaacatacactgggtgaagcagagccatggaaagagccttgagtggattggatatattaaccctaacaatggtggtactgactacaaccagaagttccagggcaaggccacattgactgtaaacaagtcctccagcacagcctacatggagctccgcagcctgacatcggaagattctgcagtctattactgtgcaagaggggggttttatggcaattacggggcttactatggtatggacttctggggtcaaggaacctcagtcaccgtctcctca

SEQ ID NO: 34 - protein, 107 aa, Artificial
  Light chain variable domain of antibody 2-J9-A
DIQMTQSPASLSASVGETVTITCRTSENIYSHLAWYQQTQGKSPQLLVYNTKTLAEGVPSRFTGSGSGTQFSLKINSLQPEDFGSYYCQHHYGIPPEFGGGTKLEIK

SEQ ID NO: 35 - DNA, 321 bp, Artificial
  Light chain variable domain of antibody 2-J9-A
gacatccagatgactcagtctccagcctccctatctgcatctgtgggagaaactgtcaccatcacatgtcgaacaagtgagaatatttatagtcatttagcatggtatcagcagacacagggaaaatctcctcagctcctggtctataatacaaaaaccttagcagaaggtgtgccatcaaggttcactggcagtggatcaggcacacagttttctctgaagatcaacagcctgcagcctgaagattttgggagttattactgtcaacatcattatggtattcctccggagttcggtggaggcaccaagctggaaatcaaa

SEQ ID NO: 36 - protein, 8 aa, Artificial
  HC-CDR1 of antibody 2-J9-A
GYTFTDYN

SEQ ID NO: 37 - protein, 8 aa, Artificial
  HC-CDR2 of antibody 2-J9-A
INPNNGGT

SEQ ID NO: 38 - protein, 17 aa, Artificial
  HC-CDR3 of antibody 2-J9-A
ARGGFYGNYGAYYGMDF

SEQ ID NO: 39 - protein, 6 aa, Artificial
  LC-CDR1 of antibody 2-J9-A
ENIYSH

SEQ ID NO: 40 - protein, 3 aa, Artificial
  LC-CDR2 of antibody 2-J9-A
NTK

SEQ ID NO: 41 - protein, 9 aa, Artificial
  LC-CDR3 of antibody 2-J9-A
QHHYGIPPE

SEQ ID NO: 42 - protein, 120 aa, Artificial
  Heavy chain variable domain of antibody 2-O12-A
EVQLQQSGPELVKPGASVKISCKASGYTFTDYYMNWVKQSHGKSLEWIGDINSNNGGTTYDQKFKGKATLTVDRSSSTAYMELRSLTSEDSAVYYCARRGDGHFGGFTYWGQGTLVTVSA

SEQ ID NO: 43 - DNA, 360 bp, Artificial
  Heavy chain variable domain of antibody 2-O12-A
gaggtccagctgcaacaatctggacctgaactggtgaagcctggggcctcagtgaagatatcctgtaaggcttctggatacacgttcactgactactacatgaactgggtgaagcagagccatggaaagagccttgagtggattggagatattaattctaacaatggtggtactacctacgaccagaagttcaagggcaaggccactttgactgtagacaggtcctccagcacagcctacatggagctccgcagcctgacatctgaggactctgcagtctattactgtgcaaggagaggggatggtcactttggggggtttacttattggggccaagggactctggtcactgtctctgca

SEQ ID NO: 44 - protein, 111 aa, Artificial
  Light chain variable domain of antibody 2-O12-A
DIVLTQSPASLAVSLGQRATISCRASESVDNSGISFMNWFQQKPGQPPKLLIYAASNQGSGVPARFSGSGSGTDFSLNIHPMEEDDTAMYFCQQSKEVPWTFGGGTKLEIK

SEQ ID NO: 45 - DNA, 333 bp, Artificial
  Light chain variable domain of antibody 2-O12-A
gacattgtgctgacccaatctccagcttctttggctgtgtctctagggcagagggccaccatctcctgcagagccagcgaaagtgttgataattctggcattagttttatgaactggttccaacagaaaccaggacagccacccaaactcctcatctatgctgcatccaaccaaggatccggggtccctgccaggtttagtggcagtgggtctgggacagacttcagcctcaacatccatcctatggaggaggatgatactgcaatgtatttctgtcagcaaagtaaggaggttccgtggacgttcggtggaggcaccaagctggaaatcaaa

SEQ ID NO: 46 - protein, 8 aa, Artificial
  HC-CDR1 of antibody 2-O12-A
GYTFTDYY

SEQ ID NO: 47 - protein, 8 aa, Artificial
  HC-CDR2 of antibody 2-O12-A
INSNNGGT

SEQ ID NO: 48 - protein, 13 aa, Artificial
  HC-CDR3 of antibody 2-O12-A
ARRGDGHFGGFTY

SEQ ID NO: 49 - protein, 10 aa, Artificial
  LC-CDR1 of antibody 2-O12-A
ESVDNSGISF

SEQ ID NO: 50 - protein, 3 aa, Artificial
  LC-CDR2 of antibody 2-O12-A
AAS

SEQ ID NO: 51 - protein, 9 aa, Artificial
  LC-CDR3 of antibody 2-O12-A
QQSKEVPWT

SEQ ID NO: 52 - protein, 114 aa, Artificial
  Heavy chain variable domain of antibody 2-P2-A
EVQLQQSGAELVRPGSSVKMSCKTSGYTFTSYGINWVKQRPGQGLEWIGYIYIGNGYTEYNEKFKGKATLTSDTSSSTAYMQLSSLTSEDSAIYFCARGGVDYWGQGTTLTVSS

SEQ ID NO: 53 - DNA, 342 bp, Artificial
  Heavy chain variable domain of antibody 2-P2-A
gaggtccagcttcagcagtctggagctgagctggtgaggcctgggtcctcagtgaagatgtcctgcaagacttctggatatacattcacaagctacggtataaactgggtgaagcagaggcctggacagggcctggaatggattggatatatttatattggaaatggttatactgaatacaatgagaagttcaagggtaaggccacactgacttcagacacatcctccagcacagcctacatgcagctcagcagcctgacatctgaggactctgcaatctatttctgtgcaagaggaggggttgactactggggccaaggcaccactctcacagtctcctca

SEQ ID NO: 54 - protein, 112 aa, Artificial
  Light chain variable domain of antibody 2-P2-A
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPYTFGGGTKLEIK

SEQ ID NO: 55 - DNA, 336 bp, Artificial
  Light chain variable domain of antibody 2-P2-A
gatgttttgatgacccaaactccactctccctgcctgtcagtcttggagatcaagcctccatctcttgcagatctagtcagagtattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgtacacgttcggaggggggaccaagctggaaataaaa

SEQ ID NO: 56 - protein, 8 aa, Artificial
  HC-CDR1 of antibody 2-P2-A
GYTFTSYG

SEQ ID NO: 57 - protein, 8 aa, Artificial
  HC-CDR2 of antibody 2-P2-A
IYIGNGYT

SEQ ID NO: 58 - protein, 7 aa, Artificial
  HC-CDR3 of antibody 2-P2-A
ARGGVDY

SEQ ID NO: 59 - protein, 11 aa, Artificial
  LC-CDR1 of antibody 2-P2-A
QSIVHSNGNTY

SEQ ID NO: 60 - protein, 3 aa, Artificial
  LC-CDR2 of antibody 2-P2-A
KVS

SEQ ID NO: 61 - protein, 9 aa, Artificial
  LC-CDR3 of antibody 2-P2-A
FQGSHVPYT

SEQ ID NO: 62 - protein, 124 aa, Artificial
  Heavy chain variable domain of antibody 3-E13-A
EVQLQQSGPELVKPGASVKMSCKASGYTFTDYNIHWVKQSHGKSLEWIGYINPNNGGTDYNQKFQGKATLTVNKSSSTAYMELRSLTSEDFAVYYCARGGFYGNYGAYYGMDFWGQGTSVTVSS

SEQ ID NO: 63 - DNA, 372 bp, Artificial
  Heavy chain variable domain of antibody 3-E13-A
gaggtccagctgcaacagtctggacctgagctggtgaagcctggggcttcagtgaagatgtcctgcaaggcttctggatacacattcactgactacaacatacactgggtgaaacagagccatggaaagagccttgagtggattggatatattaaccctaacaatggtggtactgactacaaccagaagttccagggcaaggccacattgactgtaaacaagtcctccagcacagcctacatggagctccgcagcctgacatcggaagattttgcagtctattactgtgcaagaggggggttttatggcaattacggggcttactatggtatggacttctggggtcaaggaacctcagtcaccgtctcctca

SEQ ID NO: 64 - protein, 107 aa, Artificial
  Light chain variable domain of antibody 3-E13-A
DIQMTQSPASLSASVGETVTITCRTSENIYSHLAWYQQTQGKSPQLLVYNAKTLAEGVPSRFSGSGSGTHFSLKINSLQPEDFGSYYCQHHYGIPPEFGGGTKLEIK

SEQ ID NO: 65 - DNA, 321 bp, Artificial
  Light chain variable domain of antibody 3-E13-A
gacatccagatgactcagtctccagcctccctatctgcatctgtgggagaaactgtcaccatcacatgtcgaacaagtgagaatatttatagtcatttagcatggtatcagcagacacagggaaaatctcctcagctcctggtctataatgcaaaaaccttagcagaaggtgtgccatcaaggttcagtggcagtggatcaggcacacatttttctctgaagatcaacagcctgcagcctgaagattttgggagttattactgtcaacatcattatggtattcctccggagttcggtggaggcaccaagctggaaatcaaa

SEQ ID NO: 66 - protein, 8 aa, Artificial
  HC-CDR1 of antibody 3-E13-A
GYTFTDYN

SEQ ID NO: 67 - protein, 8 aa, Artificial
  HC-CDR2 of antibody 3-E13-A
INPNNGGT

SEQ ID NO: 68 - protein, 17 aa, Artificial
  HC-CDR3 of antibody 3-E13-A
ARGGFYGNYGAYYGMDF

SEQ ID NO: 69 - protein, 6 aa, Artificial
  LC-CDR1 of antibody 3-E13-A
ENIYSH

SEQ ID NO: 70 - protein, 3 aa, Artificial
  LC-CDR2 of antibody 3-E13-A
NAK

SEQ ID NO: 71 - protein, 9 aa, Artificial
  LC-CDR3 of antibody 3-E13-A
QHHYGIPPE

SEQ ID NO: 72 - protein, 118 aa, Artificial
  Heavy chain variable domain of antibody 3-P7-A
QAYLQQSGAELVRPGASVKMSCKASGYTFTSYSMHWVKQTPRQGLEWIGAIYPGNDDTSYNQKFKGKATLTVDKSSSTAYMQLSSLTSEDSAVYFCTRDYTNYPFDYWGQGTTLTVSS

SEQ ID NO: 73 - DNA, 354 bp, Artificial
  Heavy chain variable domain of antibody 3-P7-A
caggcttatctacagcagtctggggctgagctggtgaggcctggggcctcagtgaagatgtcctgcaaggcttctggctacacatttaccagttacagtatgcactgggtaaagcagacacctagacagggcctggaatggattggagctatttatccaggaaatgatgatacttcctacaatcagaagttcaagggcaaggccacactgactgtcgacaaatcctccagcacagcctacatgcagctcagcagcctcacatctgaggactctgcggtctatttctgtacaagagactatactaactacccttttgactactggggccaaggcaccactctcacagtctcctca

SEQ ID NO: 74 - protein, 111 aa, Artificial
  Light chain variable domain of antibody 3-P7-A
DIVLTQSPASLPVSLGQRATISCRASKSVSTSGYSYMHWYQQRPGQPPKLLIYLASNLESGVPARFSASGSGTDFTLNIHPVEEEDAATYYCQHSRELPPTFGGGTKVEIK

SEQ ID NO: 75 - DNA, 333 bp, Artificial
  Light chain variable domain of antibody 3-P7-A
gacattgtgctgacacagtctcctgcttccttacctgtatctctggggcagagggccaccatctcatgcagggccagcaaaagtgtcagtacatctggctatagttatatgcactggtaccaacagagaccaggacagccacccaaactcctcatctatcttgcatccaacctagaatctggggtccctgccaggttcagtgccagtgggtctgggacagacttcaccctcaacatccatcctgtggaggaggaggatgctgcaacctattactgtcagcacagtagggagcttcctccgacgttcggtggaggcaccaaggtggagatcaaa

SEQ ID NO: 76 - protein, 8 aa, Artificial
  HC-CDR1 of antibody 3-P7-A
GYTFTSYS

SEQ ID NO: 77 - protein, 8 aa, Artificial
  HC-CDR2 of antibody 3-P7-A
IYPGNDDT

SEQ ID NO: 78 - protein, 11 aa, Artificial
  HC-CDR3 of antibody 3-P7-A
TRDYTNYPFDY

SEQ ID NO: 79 - protein, 10 aa, Artificial
  LC-CDR1 of antibody 3-P7-A
KSVSTSGYSY

SEQ ID NO: 80 - protein, 3 aa, Artificial
  LC-CDR2 of antibody 3-P7-A
LAS

SEQ ID NO: 81 - protein, 9 aa, Artificial
  LC-CDR3 of antibody 3-P7-A
QHSRELPPT

SEQ ID NO: 82 - protein, 122 aa, Artificial
  Heavy chain variable domain of antibody 4-A15-A
QVQLQQSGAELVKPGASVKISCKASGNAFSSYWMNWVKQRPGKGLEWIGQIYPGDGDTNFNGRFKGKATLTADISSTTAYMLLSSLTSEDSAVYFCASGGYGSSYERDFDYWGQGTTLTVSS

SEQ ID NO: 83 - DNA, 366 bp, Artificial
  Heavy chain variable domain of antibody 4-A15-A
caggttcagctgcagcagtctggggctgagctggtgaagcctggggcctcagtgaagatttcctgcaaagcttcaggcaacgcattcagtagttactggatgaactgggtgaagcagaggcctggaaagggtcttgagtggattggacagatttatcctggagatggtgatactaacttcaacggaaggttcaagggcaaggccacactgactgcagacatatcctccaccacagcctacatgctgctcagcagcctgacctctgaggactctgcggtctatttctgtgcaagcgggggctacggtagtagctacgagagggactttgactactggggccaaggcaccactctcacagtctcctca

SEQ ID NO: 84 - protein, 107 aa, Artificial
  Light chain variable domain of antibody 4-A15-A
DIQMNQSPSSLSASLGDTITITCHASQNINVWLSWYQQKPGNIPKLLIYKASNLHTGVPSRFSGSGSGTGFTLTISSLQPEDIATYYCQQGHSYPLTFGGGTKLEIK

SEQ ID NO: 85 - DNA, 321 bp, Artificial
  Light chain variable domain of antibody 4-A15-A
gacatccagatgaaccagtctccatccagtctgtctgcatcccttggagacacaattaccatcacttgccatgccagtcagaacattaatgtttggttaagctggtaccagcagaaaccaggaaatattcctaaactattgatctataaggcttccaacttgcacacaggcgtcccatcaaggtttagtggcagtggatctggaacaggtttcacattaaccatcagcagcctgcagcctgaagacattgccacttactactgtcaacagggtcacagttatcctctcacgttcggaggggggaccaagctggaaataaaa

SEQ ID NO: 86 - protein, 8 aa, Artificial
  HC-CDR1 of antibody 4-A15-A
GNAFSSYW

SEQ ID NO: 87 - protein, 8 aa, Artificial
  HC-CDR2 of antibody 4-A15-A
IYPGDGDT

SEQ ID NO: 88 - protein, 15 aa, Artificial
  HC-CDR3 of antibody 4-A15-A
ASGGYGSSYERDFDY

SEQ ID NO: 89 - protein, 6 aa, Artificial
  LC-CDR1 of antibody 4-A15-A
QNINVW

SEQ ID NO: 90 - protein, 3 aa, Artificial
  LC-CDR2 of antibody 4-A15-A
KAS

SEQ ID NO: 91 - protein, 9 aa, Artificial
  LC-CDR3 of antibody 4-A15-A
QQGHSYPLT

SEQ ID NO: 92 - protein, 123 aa, Artificial
  Heavy chain variable domain of antibody 4-C3-A
EVQLQQSGPELVKPGASVKISCKASGYSFTGYYMHWVKQSHGNILDWIGYIYPYNGVSSYNQKFKGKATLTVDKSSSTAYMELRSLTSEASAVYYCARAYGSSYDYYYGLDYWGQGTSVTVSS

SEQ ID NO: 93 - DNA, 369 bp, Artificial
  Heavy chain variable domain of antibody 4-C3-A
gaggttcagctgcagcagtctggacctgagctggtgaagcctggggcttcagtgaagatatcctgcaaggcctctggttactcattcactggctactacatgcactgggtgaagcagagccatggaaatatcctcgattggattggatatatttatccttacaatggtgtttctagctacaaccagaaattcaagggcaaggccacattgactgtagacaagtcctctagcacagcctacatggagctccgcagcctgacatctgaggcctctgcagtctattactgtgcaagagcctacggcagtagctacgattattactatggtttggactactggggtcaaggaacctcagtcaccgtctcctca

SEQ ID NO: 94 - protein, 111 aa, Artificial
  Light chain variable domain of antibody 4-C3-A
DIVLTQSPASLAVSLGQRATISCKASQSVDYDGAGYMNWYQQKPGQPPKLLISSASNLQSGIPARFSGSGSGTDFTLNIHPVEEEDAATYYCQQSNDDPYTFGGGTKLEIK

SEQ ID NO: 95 - DNA, 333 bp, Artificial
  Light chain variable domain of antibody 4-C3-A
gacattgtactgacccaatctccagcttctttggctgtgtctctggggcagagggccaccatctcctgcaaggccagccaaagtgttgattatgatggtgctggttatatgaactggtaccaacagaaaccaggacagccacccaaactcctcatctcttctgcatccaatctacaatctgggatcccagccaggtttagtggcagtgggtctgggacagacttcaccctcaacatccatcctgtggaggaggaggatgctgcaacctattactgtcagcaaagtaatgacgatccgtacacgttcggcggggggaccaagctggaaataaaa

SEQ ID NO: 96 - protein, 8 aa, Artificial
  HC-CDR1 of antibody 4-C3-A
GYSFTGYY

SEQ ID NO: 97 - protein, 8 aa, Artificial
  HC-CDR2 of antibody 4-C3-A
IYPYNGVS

SEQ ID NO: 98 - protein, 16 aa, Artificial
  HC-CDR3 of antibody 4-C3-A
ARAYGSSYDYYYGLDY

SEQ ID NO: 99 - protein, 10 aa, Artificial
  LC-CDR1 of antibody 4-C3-A
QSVDYDGAGY

SEQ ID NO: 100 - protein, 3 aa, Artificial
  LC-CDR2 of antibody 4-C3-A
SAS

SEQ ID NO: 101 - protein, 9 aa, Artificial
  LC-CDR3 of antibody 4-C3-A
QQSNDDPYT

SEQ ID NO: 102 - protein, 117 aa, Artificial
  Heavy chain variable domain of antibody 4-K13-A
QAYLQQSGAELVRPGASVKMSCKASGYTFTSYTVHWVKQTPRQGLEWIGAIYPGNVDTSYNQNFKGKATLTVDKSSSTAYMQLSSLTSEDSAVYFCARDGYYAMDYWGQGTSVTVSS

SEQ ID NO: 103 - DNA, 351 bp, Artificial
  Heavy chain variable domain of antibody 4-K13-A
caggcttatctacagcagtctggggctgagctggtgaggcctggggcctcagtgaagatgtcctgcaaggcttctggctacacatttaccagttacactgtgcactgggtaaagcagacacctagacagggcctggaatggattggagctatttatccaggaaatgttgatacttcctacaatcagaatttcaagggcaaggccacactgactgtagacaaatcctccagcacagcctacatgcagctcagcagcctgacatctgaagactctgcggtctatttctgtgcaagagatggttactatgctatggactactggggtcaaggaacctcagtcaccgtctcctca

SEQ ID NO: 104 - protein, 113 aa, Artificial
  Light chain variable domain of antibody 4-K13-A
DIVLSQSPSSLPVSVGEKVTMSCKSSQSLLYSSNQKNYLAWYQQKPGQSPKLLIYWASTRESGVPDRFTGSGSGTDFTLTISSVKAEDLAVYYCQQYYSYPWTFGGGTKLEIK

SEQ ID NO: 105 - DNA, 339 bp, Artificial
  Light chain variable domain of antibody 4-K13-A
gacattgtgttgtcacagtctccatcctccctacctgtgtcagttggagagaaggttactatgagctgcaagtccagtcagagccttttatatagtagcaatcaaaagaactacttggcctggtaccagcagaaaccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcagcaatattatagctatccgtggacgttcggtggaggcaccaagctggaaatcaaa

SEQ ID NO: 106 - protein, 8 aa, Artificial
  HC-CDR1 of antibody 4-K13-A
GYTFTSYT

SEQ ID NO: 107 - protein, 8 aa, Artificial
  HC-CDR2 of antibody 4-K13-A
IYPGNVDT

SEQ ID NO: 108 - protein, 10 aa, Artificial
  HC-CDR3 of antibody 4-K13-A
ARDGYYAMDY

SEQ ID NO: 109 - protein, 12 aa, Artificial
  LC-CDR1 of antibody 4-K13-A
QSLLYSSNQKNY

SEQ ID NO: 110 - protein, 3 aa, Artificial
  LC-CDR2 of antibody 4-K13-A
WAS

SEQ ID NO: 111 - protein, 9 aa, Artificial
  LC-CDR3 of antibody 4-K13-A
QQYYSYPWT

SEQ ID NO: 112 - protein, 117 aa, Artificial
  Heavy chain variable domain of antibody 4-L4-A
QAYLQQSGAELVRPGASVKMSCKASGYTFTSYTVHWVKQTPRQGLEWIGGIYPGNVDTSYNQNFKGKATLTVDKSSSTAYMQLSSLTSEDSAVYFCARDGYYAMDYWGQGTSVTVSS

SEQ ID NO: 113 - DNA, 351 bp, Artificial
  Heavy chain variable domain of antibody 4-L4-A
caggcttatctacagcagtctggggctgagctggtgaggcctggggcctcagtgaagatgtcctgcaaggcttctggctacacatttaccagttacactgtgcactgggtaaagcagacacctagacagggcctggaatggattggaggtatttatccaggaaatgttgatacttcctacaatcagaatttcaagggcaaggccacactgactgtagacaaatcctccagcacagcctacatgcagctcagcagcctgacatctgaagactctgcggtctatttctgtgcaagagatggttactatgctatggactactggggtcaaggaacctcagtcaccgtctcctca

SEQ ID NO: 114 - protein, 113 aa, Artificial
  Light chain variable domain of antibody 4-L4-A
DIVLSQSPSSLPVSVGEKVTMSCKSSQSLLYSSNQKNYLGWYQQKPGQSPKLLIYWASTRESGVPDRFTGSGSGTDFTLTISSVKAEDLAVYYCQQYYSYPWTFGGGTKLEIK

SEQ ID NO: 115 - DNA, 339 bp, Artificial
  Light chain variable domain of antibody 4-L4-A
gacattgtgttgtcacagtctccatcctccctacctgtgtcagttggagagaaggttactatgagctgcaagtccagtcagagccttttatatagtagcaatcaaaagaactacttgggctggtaccagcagaaaccagggcagtctcctaaactgctgatttactgggcatccactagggaatctggggtccctgatcgcttcacaggcagtggatctgggacagattttactctcaccatcagtagtgtgaaggctgaagacctggcagtttattactgtcagcaatattatagctatccgtggacgttcggtggaggcaccaagctggaaatcaaa

SEQ ID NO: 116 - protein, 8 aa, Artificial
  HC-CDR1 of antibody 4-L4-A
GYTFTSYT

SEQ ID NO: 117 - protein, 8 aa, Artificial
  HC-CDR2 of antibody 4-L4-A
IYPGNVDT

SEQ ID NO: 118 - protein, 10 aa, Artificial
  HC-CDR3 of antibody 4-L4-A
ARDGYYAMDY

SEQ ID NO: 119 - protein, 12 aa, Artificial
  LC-CDR1 of antibody 4-L4-A
QSLLYSSNQKNY

SEQ ID NO: 120 - protein, 3 aa, Artificial
  LC-CDR2 of antibody 4-L4-A
WAS

SEQ ID NO: 121 - protein, 9 aa, Artificial
  LC-CDR3 of antibody 4-L4-A
QQYYSYPWT

SEQ ID NO: 122 - protein, 123 aa, Artificial
  Heavy chain variable domain of antibody 5-H22-A
QVQLQQSGAELVRPGASVTLSCKASGYTFTDYEMHWMKQTPVHGLEWIGAIDLETGGTVYNQKFKGKAILTADKSSNTAYMALRSLTSEDSAVYYCTREFPHYYGSRYGFPYWGQGTLVTVSA

SEQ ID NO: 123 - DNA, 369 bp, Artificial
  Heavy chain variable domain of antibody 5-H22-A
caggttcaactgcagcagtctggggctgagctggtgaggcctggggcttcagtgacgctgtcctgcaaggcttcgggctacacatttactgactatgaaatgcactggatgaaacagacacctgtgcatggcctggaatggattggagctattgatcttgaaactggtggtactgtctacaatcagaagttcaagggcaaggccatactgactgcagacaaatcctccaacacagcctacatggcgctccgcagtctgacatctgaggactctgccgtctattactgtacaagagagttcccccattactacggtagtaggtacggttttccttactggggccaggggactctggtcactgtctctgca

SEQ ID NO: 124 - protein, 106 aa, Artificial
  Light chain variable domain of antibody 5-H22-A
QIVLTQSPAIMSASPGEKVTITCSASSSVSYMHWLQQKPGTSPKLWIYSTSNLASGVPARFSGSGSGTSYSLTISRMEAEDAATYYCQQRSSYPYTFGGGTKLEIK

SEQ ID NO: 125 - DNA, 318 bp, Artificial
  Light chain variable domain of antibody 5-H22-A
caaattgttctcacccagtctccagcaatcatgtctgcatctccaggggagaaggtcaccataacctgtagtgccagctcaagtgtaagttacatgcactggttacagcagaagccaggcacttctcccaaactctggatttatagcacatccaacctggcttctggagtccctgctcgcttcagtggcagtggatctgggacctcttactctctcacaatcagccgaatggaggctgaagatgctgccacttattactgccagcaaaggagtagttacccgtacacgttcggaggggggaccaagctggaaataaaa

SEQ ID NO: 126 - protein, 8 aa, Artificial
  HC-CDR1 of antibody 5-H22-A
GYTFTDYE

SEQ ID NO: 127 - protein, 8 aa, Artificial
  HC-CDR2 of antibody 5-H22-A
IDLETGGT

SEQ ID NO: 128 - protein, 16 aa, Artificial
  HC-CDR3 of antibody 5-H22-A
TREFPHYYGSRYGFPY

SEQ ID NO: 129 - protein, 5 aa, Artificial
  LC-CDR1 of antibody 5-H22-A
SSVSY

SEQ ID NO: 130 - protein, 3 aa, Artificial
  LC-CDR2 of antibody 5-H22-A
STS

SEQ ID NO: 131 - protein, 9 aa, Artificial
  LC-CDR3 of antibody 5-H22-A
QQRSSYPYT

SEQ ID NO: 132 - protein, 118 aa, Artificial
  Heavy chain variable domain of antibody 5-P24-A
QAYLQQSGAELVRPGASVKMSCKASGYTFTSYSMHWVKQTPRQGLEWIGAIYPGNGDTSYTQKFKGKATLTVDKSSSTAYMQLSSLTSEDSAVYFCARDYSNYPFDYWGQGTTLTVSS

SEQ ID NO: 133 - DNA, 354 bp, Artificial
  Heavy chain variable domain of antibody 5-P24-A
caggcttatctacagcagtctggggctgagctggtgaggcctggggcctcagtgaagatgtcctgcaaggcttctggctacacatttaccagttacagtatgcactgggtaaagcagacacctagacagggcctggaatggattggagctatttatccaggaaatggtgatacttcctacactcagaagttcaagggcaaggccacactgactgtagacaaatcctccagcacagcctacatgcagctcagcagcctgacatctgaagactctgcggtctatttctgtgcaagagactatagtaactacccttttgactactggggccaaggcaccactctcacagtctcctca

SEQ ID NO: 134 - protein, 113 aa, Artificial
  Light chain variable domain of antibody 5-P24-A
DIVMTQSPSSLAMSVGQKVTMSCKSSQSLLNSSNQKNYLAWYQQKPGQSPKLLVYFASTRESGVPDRFIGSGSGTDFTLTISSVQAEDLADYFCQQHYSTPLTFGAGTKLELK

SEQ ID NO: 135 - DNA, 339 bp, Artificial
  Light chain variable domain of antibody 5-P24-A
gacattgtgatgacacagtctccatcctccctggctatgtcagtaggacagaaggtcactatgagctgcaagtccagtcagagccttttaaatagtagcaatcaaaagaactatttggcctggtaccagcagaaaccaggacagtctcctaaacttctggtatactttgcatccactagggaatctggggtccctgatcgcttcataggcagtggatctgggacagatttcactcttaccatcagcagtgtgcaggctgaagacctggcagattacttctgtcagcaacattatagcactccgctcacgttcggtgctgggaccaagctggagctgaaa

SEQ ID NO: 136 - protein, 8 aa, Artificial
  HC-CDR1 of antibody 5-P24-A
GYTFTSYS

SEQ ID NO: 137 - protein, 8 aa, Artificial
  HC-CDR2 of antibody 5-P24-A
IYPGNGDT

SEQ ID NO: 138 - protein, 11 aa, Artificial
  HC-CDR3 of antibody 5-P24-A
ARDYSNYPFDY

SEQ ID NO: 139 - protein, 12 aa, Artificial
  LC-CDR1 of antibody 5-P24-A
QSLLNSSNQKNY

SEQ ID NO: 140 - protein, 3 aa, Artificial
  LC-CDR2 of antibody 5-P24-A
FAS

SEQ ID NO: 141 - protein, 9 aa, Artificial
  LC-CDR3 of antibody 5-P24-A
QQHYSTPLT

SEQ ID NO: 142 - protein, 118 aa, Artificial
  Heavy chain variable domain of antibody 6-O12-A
QVQLQQPGTELVKPGASVKLSCKASGYTFTSYWMHWVKQRPGQGLEWIGNINPSNGGTNYNEKFRSKATLTVDKSSSTAYMQLSSLTSEDSAVYYCARLGWFLPLGSWGQGTTLTVSS

SEQ ID NO: 143 - DNA, 354 bp, Artificial
  Heavy chain variable domain of antibody 6-O12-A
caggtccaactgcagcagcctgggactgaactggtgaagcctggggcttcagtgaagctgtcctgcaaggcttctggctacaccttcaccagctactggatgcactgggtgaagcagaggcctggacaaggccttgagtggattggaaatattaatcctagcaatggtggtactaactacaatgagaagttcaggagcaaggccacactgactgtagacaaatcctccagcacagcctacatgcagctcagcagcctgacatctgaggactctgcggtctattattgtgcaagactgggatggtttctaccccttgggtcctggggccaaggcaccactctcacagtctcctca

SEQ ID NO: 144 - protein, 106 aa, Artificial
  Light chain variable domain of antibody 6-O12-A
QIVLTQSPAIMSASLGEVITLTCSASSSVSYMHWYQQKSGTSPKLLIYSTSNLASGVPSRFSGSGSGTFYSLTISSVEAEDAADYYCHQWSGYMYTFGGGTKLEIK

SEQ ID NO: 145 - DNA, 318 bp, Artificial
  Light chain variable domain of antibody 6-O12-A
caaattgttctcacccagtctccagcaatcatgtctgcatctctaggggaggtgatcaccctaacctgcagtgccagctcgagtgtaagttacatgcactggtaccagcagaagtcaggcacttctcccaaactcttgatttatagcacatccaacctggcttctggagtcccttctcgcttcagtggcagtgggtctgggaccttttattctctcacaatcagcagtgtggaggctgaagatgctgccgattattactgccatcagtggagtggttatatgtacacattcggaggggggaccaaactggaaataaaa

SEQ ID NO: 146 - protein, 8 aa, Artificial
  HC-CDR1 of antibody 6-O12-A
GYTFTSYW

SEQ ID NO: 147 - protein, 8 aa, Artificial
  HC-CDR2 of antibody 6-O12-A
INPSNGGT

SEQ ID NO: 148 - protein, 11 aa, Artificial
  HC-CDR3 of antibody 6-O12-A
ARLGWFLPLGS

SEQ ID NO: 149 - protein, 5 aa, Artificial
  LC-CDR1 of antibody 6-O12-A
SSVSY

SEQ ID NO: 150 - protein, 3 aa, Artificial
  LC-CDR2 of antibody 6-O12-A
STS

SEQ ID NO: 151 - protein, 9 aa, Artificial
  LC-CDR3 of antibody 6-O12-A
HQWSGYMYT

SEQ ID NO: 152 - protein, 125 aa, Artificial
  Heavy chain variable domain of antibody 8-N24-A
QVQLQQPGAELVKPGASVKVSCKASGYTFTSYWMHWVKQRPGQGLEWIGRIHPSDSDTNYYQKFKGKATLSVDKSSSTAYMQLSSLTSEDSAVYYCAIPLYSYDNSGGYYPMDYWGQGTSVTVSS

SEQ ID NO: 153 - DNA, 375 bp, Artificial
  Heavy chain variable domain of antibody 8-N24-A
caggtccaactgcagcagcctggggctgaactggtgaagcctggggcttcagtgaaggtgtcctgcaaggcttctggctacaccttcaccagctactggatgcactgggtgaagcagaggcctggccaaggccttgagtggattggaaggattcatccttctgatagtgatactaactactatcaaaagttcaagggcaaggccacattgtctgtagacaaatcctccagcacagcctacatgcagctcagcagcctgacatctgaggactctgcggtctattactgtgcaattcccctttattcctacgataatagcggaggttactatcctatggactactggggtcaaggaacctcagtcaccgtctcctca

SEQ ID NO: 154 - protein, 107 aa, Artificial
  Light chain variable domain of antibody 8-N24-A
DIVMTQSQKFMSTSVGDRVTITCKASQNVRTAVAWYQQKPGQSPKKLIYLASNRHTGVPDRFTGSGSGTDFTLAISNVQSEDLADYFCLQHWNYPFTFGSGTRLEIK

SEQ ID NO: 155 - DNA, 321 bp, Artificial
  Light chain variable domain of antibody 8-N24-A
gacattgtgatgacccagtctcaaaaattcatgtccacatcagtaggagacagggtcaccatcacctgcaaggccagtcagaatgtccgtactgctgtagcctggtatcaacagaaaccagggcagtctcctaaaaaactgatttacttggcatccaaccggcacactggagtccctgatcgcttcacaggcagtggatctgggacagatttcactctcgccattagcaatgtgcaatctgaagacctggcagattatttctgtctgcaacattggaattatccattcacgttcggctcggggacaaggttggaaataaaa

SEQ ID NO: 156 - protein, 8 aa, Artificial
  HC-CDR1 of antibody 8-N24-A
GYTFTSYW

SEQ ID NO: 157 - protein, 8 aa, Artificial
  HC-CDR2 of antibody 8-N24-A
IHPSDSDT

SEQ ID NO: 158 - protein, 18 aa, Artificial
  HC-CDR3 of antibody 8-N24-A
AIPLYSYDNSGGYYPMDY

SEQ ID NO: 159 - protein, 6 aa, Artificial
  LC-CDR1 of antibody 8-N24-A
QNVRTA

SEQ ID NO: 160 - protein, 3 aa, Artificial
  LC-CDR2 of antibody 8-N24-A
LAS

SEQ ID NO: 161 - protein, 9 aa, Artificial
  LC-CDR3 of antibody 8-N24-A
LQHWNYPFT

SEQ ID NO: 162 - protein, 120 aa, Artificial
  Heavy chain variable domain of antibody 9-J11-A
QVQLQQSGAELVKPGASVKMSCMASGYTFTTYPIEWMKQIHGKSLEWIGNFHPYNDDSKYNEKFKGKATLSVEKSSSTVYLELSRSTSDDSAVYYCARGSYGPYWYFDVWGTGTTVTVSS

SEQ ID NO: 163 - DNA, 360 bp, Artificial
  Heavy chain variable domain of antibody 9-J11-A
caggttcagctgcagcagtctggggctgaactagtgaagcctggagcctcagtgaagatgtcctgcatggcttctggctacaccttcactacctatcctatagagtggatgaagcagattcatggaaagagcctagagtggattggaaattttcatccttacaatgatgattctaagtacaatgaaaagttcaagggcaaggccacattgagtgtagaaaaatcctctagcacagtctacttggagctcagccgatcaacatctgatgactctgctgtttattactgtgcaagggggtcttatggcccctactggtacttcgatgtctggggcacagggaccacggtcaccgtctcctca

SEQ ID NO: 164 - protein, 107 aa, Artificial
  Light chain variable domain of antibody 9-J11-A
DIQMTQSPASLSASVGETVTITCRASGDIHNYLVWYQQKQGKSPQLLVYNAKALADGVPSRFSGSGSGTQYSLKINSLQPDDFGSYYCQHFWSIPYTFGGGTKLEIK

SEQ ID NO: 165 - DNA, 321 bp, Artificial
  Light chain variable domain of antibody 9-J11-A
gacatccagatgactcagtctccagcctccctatctgcatctgtgggagaaactgtcaccatcacatgtcgagcaagtggggatattcacaattatttagtatggtatcagcagaaacagggaaaatctcctcagctcctggtctataatgcaaaagccttagcagatggtgtgccatcaaggttcagtggcagtggatcaggaacacaatattctctcaagatcaacagcctgcagcctgatgattttgggagttattactgtcaacatttttggagtattccgtacacgttcggaggggggaccaagctggaaataaaa

SEQ ID NO: 166 - protein, 8 aa, Artificial
  HC-CDR1 of antibody 9-J11-A
GYTFTTYP

SEQ ID NO: 167 - protein, 8 aa, Artificial
  HC-CDR2 of antibody 9-J11-A
FHPYNDDS

SEQ ID NO: 168 - protein, 13 aa, Artificial
  HC-CDR3 of antibody 9-J11-A
ARGSYGPYWYFDV

SEQ ID NO: 169 - protein, 6 aa, Artificial
  LC-CDR1 of antibody 9-J11-A
GDIHNY

SEQ ID NO: 170 - protein, 3 aa, Artificial
  LC-CDR2 of antibody 9-J11-A
NAK

SEQ ID NO: 171 - protein, 9 aa, Artificial
  LC-CDR3 of antibody 9-J11-A
QHFWSIPYT

SEQ ID NO: 172 - protein, 118 aa, Artificial
  Heavy chain variable domain of antibody 9-K4-A
QAYLQQSGAELVRPGASVKMSCKASGYTFPNYSFHWVKQTPRQGLEWIGAIYPGNDDTSYNQRFKGKATLTVDKSSSTAYMQLSSLTSEDSAVYFCARDYSNYPFDCWGQGTTLTVSS

SEQ ID NO: 173 - DNA, 354 bp, Artificial
  Heavy chain variable domain of antibody 9-K4-A
caggcttatctacagcagtctggggctgaactggtgaggcctggggcctcagtgaagatgtcctgcaaggcttctggctacacatttcccaattacagtttccactgggtaaagcagacacctagacagggcctggaatggattggagctatttatccaggaaatgatgatacttcctacaatcagaggttcaagggcaaggccacactgactgtagacaaatcctccagcacagcctacatgcagctcagcagcctgacatctgaagactctgcggtctatttctgtgcaagagactatagtaactacccttttgactgctggggccaaggcaccactctcacagtctcctca

SEQ ID NO: 174 - protein, 111 aa, Artificial
  Light chain variable domain of antibody 9-K4-A
DIVLTQSPASLAVSLGQRATISCRASKSVSTSGYSYMHWYQQKPGQPPKLLIYLASNLESGVPARFSGSGSGTDFTLNIHPVEEEDAATYYCQHSRELPPTFGGGTKLEIK

SEQ ID NO: 175 - DNA, 333 bp, Artificial
  Light chain variable domain of antibody 9-K4-A
gacattgtgctgacacagtctcctgcttccttagctgtatctctggggcagagggccaccatctcatgcagggccagcaaaagtgtcagtacatctggctatagttatatgcactggtaccaacagaaaccaggacagccacccaaactcctcatctatcttgcatccaacctagaatctggggtccctgccaggttcagtggcagtgggtctgggacagacttcaccctcaacatccatcctgtggaggaggaggatgctgcaacctattactgtcagcacagtagggagcttcctccgacgttcggtggaggcaccaagctggaaatcaaa

SEQ ID NO: 176 - protein, 8 aa, Artificial
  HC-CDR1 of antibody 9-K4-A
GYTFPNYS

SEQ ID NO: 177 - protein, 8 aa, Artificial
  HC-CDR2 of antibody 9-K4-A
IYPGNDDT

SEQ ID NO: 178 - protein, 11 aa, Artificial
  HC-CDR3 of antibody 9-K4-A
ARDYSNYPFDC

SEQ ID NO: 179 - protein, 10 aa, Artificial
  LC-CDR1 of antibody 9-K4-A
KSVSTSGYSY

SEQ ID NO: 180 - protein, 3 aa, Artificial
  LC-CDR2 of antibody 9-K4-A
LAS

SEQ ID NO: 181 - protein, 9 aa, Artificial
  LC-CDR3 of antibody 9-K4-A
QHSRELPPT

SEQ ID NO: 182 - protein, 123 aa, Artificial
  Heavy chain variable domain of antibody 9-L13-A
QVQLKESGPGLVAPSQSLSITCTVSGFSLTSYAISWVRQPPGKGLEWLGLIWTGGGTNYNSALKSRLSISKDNSKSQIFLKMNSLQTDDTARYYCARGPPLIYYGDLYYFDYWGQGTTLTVSS

SEQ ID NO: 183 - DNA, 369 bp, Artificial
  Heavy chain variable domain of antibody 9-L13-A
caggtgcagctgaaggagtcaggacctggcctggtggcgccctcacagagcctgtccatcacatgcactgtctctgggttctcattaaccagctatgctataagctgggttcgccagccaccagggaagggtctggagtggcttggactaatatggactggtggaggcacaaattataattcagctctcaaatccagactgagcatcagcaaagacaactccaagagtcaaattttcttaaaaatgaacagtctgcaaactgatgacacagccaggtactactgtgccagagggcctcctctcatctattatggtgacttgtactactttgactactggggccaaggcaccactctcacagtctcctca

SEQ ID NO: 184 - protein, 106 aa, Artificial
  Light chain variable domain of antibody 9-L13-A
DIQMTQSPSSLSASLGGKVTITCKASQDINKYIAWYQHKPGKGPRLLIHYTSTLQPGIPSRFSGSGSGRDYSFSISNLEPEDIATYYCLQYDNLWTFGGGTKLEIK

SEQ ID NO: 185 - DNA, 318 bp, Artificial
  Light chain variable domain of antibody 9-L13-A
gacatccagatgacacagtctccatcctcactgtctgcatctctgggaggcaaagtcaccatcacttgcaaggcaagccaagacattaacaagtatatagcttggtaccaacacaagcctggaaaaggtcctaggctgctcatacattacacatctacattacagccaggcatcccatcaaggttcagtggaagtgggtctgggagagattattccttcagcatcagcaacctggagcctgaagatattgcaacttattattgtctacagtatgataatctgtggacgttcggtggaggcaccaagctggaaatcaaa

SEQ ID NO: 186 - protein, 8 aa, Artificial
  HC-CDR1 of antibody 9-L13-A
GFSLTSYA

SEQ ID NO: 187 - protein, 7 aa, Artificial
  HC-CDR2 of antibody 9-L13-A
IWTGGGT

SEQ ID NO: 188 - protein, 17 aa, Artificial
  HC-CDR3 of antibody 9-L13-A
ARGPPLIYYGDLYYFDY

SEQ ID NO: 189 - protein, 6 aa, Artificial
  LC-CDR1 of antibody 9-L13-A
QDINKY

SEQ ID NO: 190 - protein, 3 aa, Artificial
  LC-CDR2 of antibody 9-L13-A
YTS

SEQ ID NO: 191 - protein, 8 aa, Artificial
  LC-CDR3 of antibody 9-L13-A
LQYDNLWT

SEQ ID NO: 192 - protein, 114 aa, Artificial
  Heavy chain variable domain of antibody 9-P9-A
DVKLVESGEGLVKPGGSLKLSCAASGFSFSNFGMSWVRQTPEKRLEWVAYISSGGDYIYYADTVKGRFTISRDNARNTLYLQMSSLKSEDTAIYYCTRGYFDYWGQGTTLTVSS

SEQ ID NO: 193 - DNA, 342 bp, Artificial
  Heavy chain variable domain of antibody 9-P9-A
gacgtgaagctggtggagtctggggaaggcttagtgaagcctggagggtccctgaaactctcctgtgcagcctccggattcagtttcagtaactttggcatgtcttgggttcgccagactccagagaagaggctggagtgggtcgcatacattagtagtggtggtgattacatctactatgcagacactgtgaagggccgattcaccatctccagagacaatgccaggaacaccctgtacctgcaaatgagcagtctgaagtctgaggacacagccatatattactgtacaaggggatactttgactactggggccaaggcaccactctcacagtctcctca

SEQ ID NO: 194 - protein, 112 aa, Artificial
  Light chain variable domain of antibody 9-P9-A
DVLMTQTPLSLPVSLGDQASISCRSSQSIVHSNGNTYLEWYLQKPGQSPKLLIYTVSNRFSGVPDRFSGSGSGTDFTLKISRVEAEDLGVYYCFQGSHVPYTFGGGTKLEMR

SEQ ID NO: 195 - DNA, 336 bp, Artificial
  Light chain variable domain of antibody 9-P9-A
gatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacacagtttccaaccgattttctggggtcccagacaggttcagtggcagtggatcggggacagatttcacactcaagatcagcagagtggaggctgaggatctgggagtttattactgctttcaaggttcacatgttccgtacacgttcggaggggggaccaagctggaaatgaga

SEQ ID NO: 196 - protein, 8 aa, Artificial
  HC-CDR1 of antibody 9-P9-A
GFSFSNFG

SEQ ID NO: 197 - protein, 8 aa, Artificial
  HC-CDR2 of antibody 9-P9-A
ISSGGDYI

SEQ ID NO: 198 - protein, 7 aa, Artificial
  HC-CDR3 of antibody 9-P9-A
TRGYFDY

SEQ ID NO: 199 - protein, 11 aa, Artificial
  LC-CDR1 of antibody 9-P9-A
QSIVHSNGNTY

SEQ ID NO: 200 - protein, 3 aa, Artificial
  LC-CDR2 of antibody 9-P9-A
TVS

SEQ ID NO: 201 - protein, 9 aa, Artificial
  LC-CDR3 of antibody 9-P9-A
FQGSHVPYT

SEQ ID NO: 202 - protein, 125 aa, Artificial; identical to SEQ ID NO: 152
  Heavy chain variable domain of antibody 10-B11-A

SEQ ID NO: 203 - DNA, 375 bp, Artificial; identical to SEQ ID NO: 153
  Heavy chain variable domain of antibody 10-B11-A

SEQ ID NO: 204 - protein, 107 aa, Artificial; identical to SEQ ID NO: 154
  Light chain variable domain of antibody 10-B11-A

SEQ ID NO: 205 - DNA, 321 bp, Artificial; identical to SEQ ID NO: 155
  Light chain variable domain of antibody 10-B11-A

SEQ ID NO: 206 - protein, 8 aa, Artificial
  HC-CDR1 of antibody 10-B11-A
GYTFTSYW

SEQ ID NO: 207 - protein, 8 aa, Artificial
  HC-CDR2 of antibody 10-B11-A
IHPSDSDT

SEQ ID NO: 208 - protein, 18 aa, Artificial
  HC-CDR3 of antibody 10-B11-A
AIPLYSYDNSGGYYPMDY

SEQ ID NO: 209 - protein, 6 aa, Artificial
  LC-CDR1 of antibody 10-B11-A
QNVRTA

SEQ ID NO: 210 - protein, 3 aa, Artificial
  LC-CDR2 of antibody 10-B11-A
LAS

SEQ ID NO: 211 - protein, 9 aa, Artificial
  LC-CDR3 of antibody 10-B11-A
LQHWNYPFT

SEQ ID NO: 212 - protein, 121 aa, Artificial
  Heavy chain variable domain of antibody 10-B13-A
QVQLQQSGAELVKPGASVKISCKASGYAFSNYWMNWVKQRPGKGLEWIGQIYPGDGDTNYNGNFKGKATLTADKSSSTAYIQLSSLTSEDSAVYFCGRGFIATVVEVFDHWGLGTTLTVSS

SEQ ID NO: 213 - DNA, 363 bp, Artificial
  Heavy chain variable domain of antibody 10-B13-A
caggttcagctgcagcagtctggggctgagctggtgaagcctggggcctcagtgaagatttcctgcaaagcttctggctacgcattcagtaactactggatgaactgggtgaagcagaggcctggaaagggtcttgagtggattggacagatttatcctggagatggtgatactaactacaacggaaacttcaagggcaaggccacactgactgcagacaaatcctccagcacagcctacattcaactcagcagcctgacctctgaggactctgcggtctatttctgtggaaggggatttattgctacggtagtagaggtttttgaccactggggcctaggcaccactctcacagtctcctca

SEQ ID NO: 214 - protein, 107 aa, Artificial
  Light chain variable domain of antibody 10-B13-A
DIQMTQTTSSLSASLGDRVTISCRASQDISNYLNWYQQKPDGTVKLLIYYTSRLQSGVPSRFSGSGTGTDYSLTISNLEQEDIATYFCQQGNTLPWTFGGGTKLEIK

SEQ ID NO: 215 - DNA, 321 bp, Artificial
  Light chain variable domain of antibody 10-B13-A
gatatccagatgacacagactacatcctccctgtctgcctctctgggagacagagtcaccatcagttgcagggcaagtcaggacattagcaattatttaaactggtatcagcagaaaccagatggaactgttaaactcctgatctattacacatcaagattacagtcaggagtcccatcaaggttcagtggcagtgggactggaacagattattctctcaccattagcaacctggagcaagaagatattgccacttacttttgccaacagggtaatacgcttccgtggacgttcggtggaggcaccaagctggaaatcaaa

SEQ ID NO: 216 - protein, 8 aa, Artificial
  HC-CDR1 of antibody 10-B13-A
GYAFSNYW

SEQ ID NO: 217 - protein, 8 aa, Artificial
  HC-CDR2 of antibody 10-B13-A
IYPGDGDT

SEQ ID NO: 218 - protein, 14 aa, Artificial
  HC-CDR3 of antibody 10-B13-A
GRGFIATVVEVFDH

SEQ ID NO: 219 - protein, 6 aa, Artificial
  LC-CDR1 of antibody 10-B13-A
QDISNY

SEQ ID NO: 220 - protein, 3 aa, Artificial
  LC-CDR2 of antibody 10-B13-A
YTS

SEQ ID NO: 221 - protein, 9 aa, Artificial
  LC-CDR3 of antibody 10-B13-A
QQGNTLPWT

SEQ ID NO: 222 - protein, 118 aa, Artificial
  Heavy chain variable domain of antibody 10-L12-A
QAYLQQSGAELVRPGASVKMSCKASGYTFTSYSMHWVKQTPRQGLEWIGAIYPGNGDTSYNQKFKGKATLTVDKSSSTAYMQLSSLTSEDSAVYFCTRDYSNYPFDYWGQGTTLTVSS

SEQ ID NO: 223 - DNA, 354 bp, Artificial
  Heavy chain variable domain of antibody 10-L12-A
caggcttatctacagcagtctggggctgagctggtgaggcctggggcctcagtgaagatgtcctgcaaggcttctggctacacatttaccagttacagtatgcactgggtaaagcagacacctagacagggcctggaatggattggagctatttatccaggaaatggtgatacttcctacaatcagaagttcaagggcaaggccacactgactgtagacaaatcctccagcacagcctacatgcagctcagcagcctgacatctgaagactctgcggtctatttctgtacaagagactatagtaactacccttttgactactggggccaaggcaccactctcacagtctcctca

SEQ ID NO: 224 - protein, 111 aa, Artificial
  Light chain variable domain of antibody 10-L12-A
DIVLTQSPASLAVSLGQRATISCRASKSVSTSGYSYLHWYQQKPGQPPKLLIYLASNLESGVPARFSGSGSGTDFTLNIHPVEEEDAATYYCQHSRELPPTFGGGTKLEIK

SEQ ID NO: 225 - DNA, 333 bp, Artificial
  Light chain variable domain of antibody 10-L12-A
gacattgtgctgacacagtctcctgcttccttagctgtatctctggggcagagggccaccatctcatgcagggccagcaaaagtgtcagtacatctggctatagttatttgcactggtaccaacagaaaccaggacagccacccaaactcctcatctatcttgcatccaacctagaatctggggtccctgccaggttcagtggcagtgggtctgggacagacttcaccctcaacatccatcctgtggaggaggaggatgctgcaacctattactgtcagcacagtagggagcttcctccgacgttcggtggaggcaccaagctggaaatcaaa

SEQ ID NO: 226 - protein, 8 aa, Artificial
  HC-CDR1 of antibody 10-L12-A
GYTFTSYS

SEQ ID NO: 227 - protein, 8 aa, Artificial
  HC-CDR2 of antibody 10-L12-A
IYPGNGDT

SEQ ID NO: 228 - protein, 11 aa, Artificial
  HC-CDR3 of antibody 10-L12-A
TRDYSNYPFDY

SEQ ID NO: 229 - protein, 10 aa, Artificial
  LC-CDR1 of antibody 10-L12-A
KSVSTSGYSY

SEQ ID NO: 230 - protein, 3 aa, Artificial
  LC-CDR2 of antibody 10-L12-A
LAS

SEQ ID NO: 231 - protein, 9 aa, Artificial
  LC-CDR3 of antibody 10-L12-A
QHSRELPPT

SEQ ID NO: 232 - protein, 120 aa, Artificial; identical to SEQ ID NO: 42
  Heavy chain variable domain of antibody 10-L24-A

SEQ ID NO: 233 - DNA, 360 bp, Artificial
  Heavy chain variable domain of antibody 10-L24-A
gaggtccagctgcaacaatctggacctgaactggtgaagcctggggcctcagtgaagatatcctgtaaggcttctggatacacgttcactgactactacatgaactgggtgaagcagagccatggaaagagccttgagtggattggagatattaattctaacaatggtggtactacctacgaccagaagttcaagggcaaggccactttgactgtagacaggtcctccagtacagcctacatggagctccgcagcctgacatctgaggactctgcagtctattactgtgcaaggagaggggatggtcactttggggggtttacttattggggccaagggactctggtcactgtctctgca

SEQ ID NO: 234 - protein, 111 aa, Artificial; identical to SEQ ID NO: 44
  Light chain variable domain of antibody 10-L24-A

SEQ ID NO: 235 - DNA, 333 bp, Artificial; identical to SEQ ID NO: 45
  Light chain variable domain of antibody 10-L24-A

SEQ ID NO: 236 - protein, 8 aa, Artificial
  HC-CDR1 of antibody 10-L24-A
GYTFTDYY

SEQ ID NO: 237 - protein, 8 aa, Artificial
  HC-CDR2 of antibody 10-L24-A
INSNNGGT

SEQ ID NO: 238 - protein, 13 aa, Artificial
  HC-CDR3 of antibody 10-L24-A
ARRGDGHFGGFTY

SEQ ID NO: 239 - protein, 10 aa, Artificial
  LC-CDR1 of antibody 10-L24-A
ESVDNSGISF

SEQ ID NO: 240 - protein, 3 aa, Artificial
  LC-CDR2 of antibody 10-L24-A
AAS

SEQ ID NO: 241 - protein, 9 aa, Artificial
  LC-CDR3 of antibody 10-L24-A
QQSKEVPWT

SEQ ID NO: 242 - protein, 118 aa, Artificial
  Heavy chain variable domain of antibody 10-O24-A
QAYLQQSGAELVRPGASVKMSCKASGYTFTSYSMHWVKQTPRQGLEWIGAIYPGNDDTSYNQKFKGKATLTVDRSSSTAYMQLSSLTSEDSAVYFCTRDYSNYPFDYWGQGTTLTVSS

SEQ ID NO: 243 - DNA, 354 bp, Artificial
  Heavy chain variable domain of antibody 10-O24-A
caggcttatctacagcagtctggggctgagctggtgaggcctggggcctcagtgaagatgtcctgcaaggcttctggctacacatttaccagttacagtatgcactgggtaaagcagacacctagacagggcctggaatggattggagctatttatccaggaaatgatgatacttcctacaatcagaagttcaagggcaaggccacactgactgtagacagatcctccagcacagcctacatgcagctcagcagcctcacatctgaagactctgcggtctatttctgtacaagagactatagtaactacccttttgactactggggccaaggcaccactctcacagtctcctca

SEQ ID NO: 244 - protein, 111 aa, Artificial
  Light chain variable domain of antibody 10-O24-A
DIVLTQSPASLAVFLGQRATISCRASKSVSTSGYSYMHWYQQRPGQPPKLLIYLASNLESGVPARFSGSGSGTDFTLNIHPVEEEDAATYYCQHSRELPPTFGGGTKLEIK

SEQ ID NO: 245 - DNA, 333 bp, Artificial
  Light chain variable domain of antibody 10-O24-A
gacattgtgctgacacagtctcctgcttccttagctgtatttctggggcagagggccaccatctcatgcagggccagcaaaagtgtcagtacatctggctatagttatatgcactggtaccaacagagaccaggacagccacccaaactcctcatctatcttgcatccaacctagaatctggggtccctgccaggttcagtggcagtgggtctgggacagacttcaccctcaacatccatcctgtggaggaggaggatgctgcaacctattactgtcagcacagtagggagcttcctccgacgttcggtggaggcaccaagctggaaatcaaa

SEQ ID NO: 246 - protein, 8 aa, Artificial
  HC-CDR1 of antibody 10-O24-A
GYTFTSYS

SEQ ID NO: 247 - protein, 8 aa, Artificial
  HC-CDR2 of antibody 10-O24-A
IYPGNDDT

SEQ ID NO: 248 - protein, 11 aa, Artificial
  HC-CDR3 of antibody 10-O24-A
TRDYSNYPFDY

SEQ ID NO: 249 - protein, 10 aa, Artificial
  LC-CDR1 of antibody 10-O24-A
KSVSTSGYSY

SEQ ID NO: 250 - protein, 3 aa, Artificial
  LC-CDR2 of antibody 10-O24-A
LAS

SEQ ID NO: 251 - protein, 9 aa, Artificial
  LC-CDR3 of antibody 10-O24-A
QHSRELPPT

SEQ ID NO: 252 - protein, 121 aa, Artificial
  Heavy chain variable domain of antibody 10-O3-A
QVQLKESGPGLVAPSQSLSITCTVSGFSLTNYAVHWVRQSPGKGLEWLGVIWSDGRTDYNAAFISRLSISKDNSKSQVFFKMNSLQADDTAIYYCVRIEGGSYVGWYFDVWGAGTTVTVSS

SEQ ID NO: 253 - DNA, 363 bp, Artificial
  Heavy chain variable domain of antibody 10-O3-A
caggtgcagctgaaggagtcaggacctggcctggtggcgccctcacagagcctgtccatcacctgcacagtctctggtttctcattaaccaactatgctgtacactgggttcgccagtctccaggaaagggtctggagtggctgggagtgatatggagtgatggaaggacagactataatgcagctttcatatctagactgagcatcagcaaggacaactccaagagccaagttttctttaaaatgaacagtctgcaagctgatgacacagccatatactactgtgtcagaatcgagggtggtagctacgttggctggtacttcgatgtctggggcgcagggaccacggtcaccgtctcctca

SEQ ID NO: 254 - protein, 106 aa, Artificial
  Light chain variable domain of antibody 10-O3-A
DIQMTQSPSSLSASLGGTVTITCKASQDINKFIAWYQHKPGKGPRLLIHYTSTLQPGIPSRFSGSGSGRDYSFSISNLEPEDIATYYCLQYDNLLTFGAGTKLELK

SEQ ID NO: 255 - DNA, 318 bp, Artificial
  Light chain variable domain of antibody 10-O3-A
gacatccagatgacacagtctccatcctcactgtctgcatctctgggaggcacagtcaccatcacttgcaaggcaagccaagacattaacaagtttatagcttggtaccaacacaagcctggaaaaggtcctaggctgctcattcattacacatctacattacagccaggcatcccatcaaggttcagtggaagtgggtctgggagagattattccttcagcatcagcaacctggagcctgaagatattgcaacttattattgtctacagtatgataatctgctcacgttcggtgctgggaccaagctggagctgaaa

SEQ ID NO: 256 - protein, 8 aa, Artificial
  HC-CDR1 of antibody 10-O3-A
GFSLTNYA

SEQ ID NO: 257 - protein, 7 aa, Artificial
  HC-CDR2 of antibody 10-O3-A
IWSDGRT

SEQ ID NO: 258 - protein, 15 aa, Artificial
  HC-CDR3 of antibody 10-O3-A
VRIEGGSYVGWYFDV

SEQ ID NO: 259 - protein, 6 aa, Artificial
  LC-CDR1 of antibody 10-O3-A
QDINKF

SEQ ID NO: 260 - protein, 3 aa, Artificial
  LC-CDR2 of antibody 10-O3-A
YTS

SEQ ID NO: 261 - protein, 8 aa, Artificial
  LC-CDR3 of antibody 10-O3-A
LQYDNLLT

SEQ ID NO: 262 - protein, 115 aa, Artificial
  Heavy chain variable domain of antibody 4-M3-A
EVQLQQSGPELVKPGDSVKISCKASGYSFTGYFMNWVMQSHGKSLEWIGRINPNNGETFYNQKFKGKATLTVDKSSDTAHMELRSLTSEDSAVYYCARGGTGTGWGQGTTLTVSS

SEQ ID NO: 263 - DNA, 345 bp, Artificial
  Heavy chain variable domain of antibody 4-M3-A
gaggttcagctgcagcagtctggacctgagctggtgaagcctggggattcagtgaagatatcctgcaaggcttctggttactcatttactggctactttatgaactgggtgatgcagagccatggaaagagccttgagtggattggacgtattaatcctaacaatggtgaaactttctacaaccagaagttcaagggcaaggccacattgactgtagacaaatcctctgacacagcccacatggagctccggagcctgacatctgaggactctgcagtctattattgtgcaagaggggggactgggacggggtggggccaaggcaccactctcacagtctcctca

SEQ ID NO: 264 - protein, 107 aa, Artificial
  Light chain variable domain of antibody 4-M3-A
DILMTQSPSSMSVSLGDTVSITCHASQGISSNIGWLQQKPGKSFKGLIYYGTNLEDGVPSRFSGSGSGADYSLTISSLESEDLADYYCIQYAQFPYTFGGGTKLEIK

SEQ ID NO: 265 - DNA, 321 bp, Artificial
  Light chain variable domain of antibody 4-M3-A
gacatcctgatgacccaatctccatcctccatgtctgtatctctgggagacacagtcagcatcacttgccatgcaagtcagggcattagcagtaatatagggtggttgcagcagaaaccagggaaatcatttaagggcctgatctattatggaaccaacttggaagatggagttccatcaaggttcagtggcagtggatctggagcagattattctctcaccatcagcagcctggaatctgaagatcttgcagactattactgtatacagtatgctcagtttccgtacacgttcggaggggggaccaaactggaaataaaa

SEQ ID NO: 266 - protein, 8 aa, Artificial
  HC-CDR1 of antibody 4-M3-A
GYSFTGYF

SEQ ID NO: 267 - protein, 8 aa, Artificial
  HC-CDR2 of antibody 4-M3-A
INPNNGET

SEQ ID NO: 268 - protein, 8 aa, Artificial
  HC-CDR3 of antibody 4-M3-A
ARGGTGTG

SEQ ID NO: 269 - protein, 6 aa, Artificial
  LC-CDR1 of antibody 4-M3-A
QGISSN

SEQ ID NO: 270 - protein, 3 aa, Artificial
  LC-CDR2 of antibody 4-M3-A
YGT

SEQ ID NO: 271 - protein, 9 aa, Artificial
  LC-CDR3 of antibody 4-M3-A
IQYAQFPYT

SEQ ID NO: 272 - protein, 114 aa, Artificial
  Heavy chain variable domain of antibody 4-N22-A
QVQLQQPGTELVKPGASVKLSCKASGYTFTNYWMYWVKQRPGQGLDWIGNINPSNGGTNYNEKFKSKATLTVDKSSSTAYMQLSSLTSEDSAVYYCARETGNYWGQGTTLTVSS

SEQ ID NO: 273 - DNA, 342 bp, Artificial
  Heavy chain variable domain of antibody 4-N22-A
caggtccaactgcagcagcctgggactgaactggtgaagcctggggcttcagtgaagctgtcctgcaaggcttctggctacaccttcaccaattactggatgtactgggtgaagcagaggcctggacaaggccttgattggattggaaatattaatcctagcaatggtggtactaactacaatgagaagttcaagagcaaggccacactgactgtagacaaatcctccagcacagcctacatgcagctcagcagcctgacatctgaggactctgcggtctattattgtgcaagagagactgggaactactggggccaaggcaccactctcacagtctcctca

SEQ ID NO: 274 - protein, 107 aa, Artificial
  Light chain variable domain of antibody 4-N22-A
DILMTQSPSSMSVSLGDTVSISCHASQGISSNIGWLQQKPGKSFKGLIYHGTNLEDGVPSRFSGSGSGADYSLTISSLESEDFADYYCVQYAQFPYTFGGGTKLEIK

SEQ ID NO: 275 - DNA, 321 bp, Artificial
  Light chain variable domain of antibody 4-N22-A
gacatcctgatgacccaatctccatcctccatgtctgtatctctgggagacacagtcagcatctcttgccatgcgagtcagggcattagcagtaatatagggtggttgcagcagaaaccagggaaatcatttaagggcctgatctatcatggaaccaacttggaagatggagttccatcaaggttcagtggcagtggatctggagcagattattctctcaccatcagcagcctggaatctgaagattttgcagactattactgtgtacagtatgctcagtttccgtacacgttcggaggggggaccaagctggaaataaaa

SEQ ID NO: 276 - protein, 8 aa, Artificial
  HC-CDR1 of antibody 4-N22-A
GYTFTNYW

SEQ ID NO: 277 - protein, 8 aa, Artificial
  HC-CDR2 of antibody 4-N22-A
INPSNGGT

SEQ ID NO: 278 - protein, 7 aa, Artificial
  HC-CDR3 of antibody 4-N22-A
ARETGNY

SEQ ID NO: 279 - protein, 6 aa, Artificial
  LC-CDR1 of antibody 4-N22-A
QGISSN

SEQ ID NO: 280 - protein, 3 aa, Artificial
  LC-CDR2 of antibody 4-N22-A
HGT

SEQ ID NO: 281 - protein, 9 aa, Artificial
  LC-CDR3 of antibody 4-N22-A
VQYAQFPYT

SEQ ID NO: 282 - protein, 124 aa, Artificial
  Heavy chain variable domain of antibody 7-B10-A
EVQLVESGGGLVQPKGSLKLSCAASGFTFNTYAMHWVRQAPGKGLEWVARIRSKSSNYARYYADSVKDRFTISRDESQSMLYLQMNNLKTEDTAMYYCVRDHDYYGSSYWFAYWGQGTLVTVSA

SEQ ID NO: 283 - DNA, 372 bp, Artificial
  Heavy chain variable domain of antibody 7-B10-A
gaggtgcagcttgttgagtctggtggaggattggtgcagcctaaaggatcattgaaactctcatgtgccgcctctggtttcaccttcaatacctatgccatgcactgggtccgccaggctccaggaaagggtttggaatgggttgctcgcataagaagtaaaagtagtaattatgcaagatattatgccgattcagtgaaagacagattcaccatttccagagatgagtcacaaagcatgctctatctgcaaatgaacaacctgaaaactgaggacacagccatgtattactgtgtgagagatcacgattactacggtagtagttactggtttgcttactggggccaagggactctggtcactgtctctgca

SEQ ID NO: 284 - protein, 112 aa, Artificial
  Light chain variable domain of antibody 7-B10-A
DVVMTQTPLSLPVSLGDQASISCSSSQSLVHNNGNTYLHWYLQKPGQSPKLLIYKVSNRFSGVPDRFSGSGSGTDFTLKISRVEAEDLGVYFCSQSTHVPWTFGGGTKLEIK

SEQ ID NO: 285 - DNA, 336 bp, Artificial
  Light chain variable domain of antibody 7-B10-A
gatgttgtgatgacccaaactccactctccctgcctgtcagtcttggagatcaagcctccatctcttgcagctctagtcagagccttgtacacaataatggaaacacctatttacattggtacctgcagaagccgggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtggacgttcggtggaggcaccaagctggaaatcaaa

SEQ ID NO: 286 - protein, 8 aa, Artificial
  HC-CDR1 of antibody 7-B10-A
GFTFNTYA

SEQ ID NO: 287 - protein, 10 aa, Artificial
  HC-CDR2 of antibody 7-B10-A
IRSKSSNYAR

SEQ ID NO: 288 - protein, 15 aa, Artificial
  HC-CDR3 of antibody 7-B10-A
VRDHDYYGSSYWFAY

SEQ ID NO: 289 - protein, 11 aa, Artificial
  LC-CDR1 of antibody 7-B10-A
QSLVHNNGNTY

SEQ ID NO: 290 - protein, 3 aa, Artificial
  LC-CDR2 of antibody 7-B10-A
KVS

SEQ ID NO: 291 - protein, 9 aa, Artificial
  LC-CDR3 of antibody 7-B10-A
SQSTHVPWT

SEQ ID NO: 292 - protein, 122 aa, Artificial
  Heavy chain variable domain of antibody 8-H5-A
QVTLKESGPGILQSSQTLSLTCSLSGFSLSTSGMGVSWVRQPSGKGLEWLAHIYWDDDKRYTPSLKSRLTISTDSSRKQVFLKITSVDTADTATYYCARRDLHDYDGWFAYWGQGTLVTVSA

SEQ ID NO: 293 - DNA, 366 bp, Artificial
  Heavy chain variable domain of antibody 8-H5-A
caggttactctgaaagagtctggccctgggatattgcagtcctcccagaccctcagtctgacttgttctttgtctgggttttcactgagcacttctggtatgggtgtgagctgggttcgtcagccttcaggaaagggtctggagtggctggcacacatttactgggatgatgacaagcgctataccccttccctgaagagccggctcacaatctccacggattcctccagaaagcaggtattcctcaagatcaccagtgtggacactgcagatactgccacatactactgtgctcgaagagatcttcatgattacgacggctggtttgcttactggggccaagggactctggtcactgtctctgca

SEQ ID NO: 294 - protein, 107 aa, Artificial
  Light chain variable domain of antibody 8-H5-A
DIVMTQSQKFMSTAVGDRVSVTCKASQNVGTNVAWYQQKPGQSPKALIYSASFRYSGVPDRFSGSGSGTDFTLTISNVQSEDLAEYFCQQYNSYPLTFGSGTKLEIK

SEQ ID NO: 295 - DNA, 321 bp, Artificial
  Light chain variable domain of antibody 8-H5-A
gacattgtgatgacccagtctcaaaaattcatgtccacagcagtaggagacagggtcagcgtcacctgcaaggccagtcaaaatgtgggtactaatgtcgcctggtatcaacagaaaccagggcaatctcctaaagcactgatttactcggcatccttccgctacagtggagtccctgatcgcttctcaggcagtggatctgggacagatttcactctcaccatcagcaatgtgcagtctgaagacttggcagagtatttctgtcagcaatataacagctatcctctcacgttcggctcggggacaaagttggaaataaaa

SEQ ID NO: 296 - protein, 10 aa, Artificial
  HC-CDR1 of antibody 8-H5-A
GFSLSTSGMG

SEQ ID NO: 297 - protein, 7 aa, Artificial
  HC-CDR2 of antibody 8-H5-A
IYWDDDK

SEQ ID NO: 298 - protein, 14 aa, Artificial
  HC-CDR3 of antibody 8-H5-A
ARRDLHDYDGWFAY

SEQ ID NO: 299 - protein, 6 aa, Artificial
  LC-CDR1 of antibody 8-H5-A
QNVGTN

SEQ ID NO: 300 - protein, 3 aa, Artificial
  LC-CDR2 of antibody 8-H5-A
SAS

SEQ ID NO: 301 - protein, 9 aa, Artificial
  LC-CDR3 of antibody 8-H5-A
QQYNSYPLT

SEQ ID NO: 302 - protein, 120 aa, Artificial
  Heavy chain variable domain of antibody 2-G20-A
EVQLQQSGPELVKPGASVKMSCKASGYTFPSYVLHWVKQKPGQGLEWIGYIYPYNDGTNYNEKFKGKATLTSDKSSSTAYMELSSLTSEDSAVYYCARGDYGDSAWFPYWGQGTLVTVSA

SEQ ID NO: 303 - DNA, 360 bp, Artificial
  Heavy chain variable domain of antibody 2-G20-A
gaggtccagctgcagcagtctggacctgagctggtaaagcctggggcttcagtgaagatgtcctgcaaggcttctggatacacattccctagttatgttttgcactgggtgaagcagaagcctgggcagggccttgagtggattggatatatttatccttacaatgatggtactaactacaatgagaagttcaaaggcaaggccacactgacttcagacaaatcctccagcacagcctacatggagctcagcagcctgacctctgaggactctgcggtctattactgtgcaaggggagactatggtgactccgcctggtttccttactggggccaagggactctggtcactgtctctgca

SEQ ID NO: 304 - protein, 111 aa, Artificial
  Light chain variable domain of antibody 2-G20-A
DIVLTQSPASLAVSLGQRATISCRASKSVSTSGYSYMHWYQQKPGQPPKLLIYLASNLKSGVPARFSGGGSGTDFTLNIHPVEEEDAATYYCQLSRELPWTFGGGTKLEIK

SEQ ID NO: 305 - DNA, 333 bp, Artificial
  Light chain variable domain of antibody 2-G20-A
gacattgtgctgacacagtctcctgcttccttagctgtatctctggggcagagggccaccatctcatgcagggccagcaaaagtgtcagtacatctggctatagttatatgcactggtaccaacagaaaccaggacagccacccaaactcctcatctatcttgcatccaacctaaaatctggggtccctgccaggttcagtggcggtgggtctgggacagacttcaccctcaacatccatcctgtggaggaggaggatgctgcaacctattactgtcagctcagtagggagcttccgtggacgttcggtggaggcaccaagctggaaatcaaa

SEQ ID NO: 306 - protein, 8 aa, Artificial
  HC-CDR1 of antibody 2-G20-A
GYTFPSYV

SEQ ID NO: 307 - protein, 8 aa, Artificial
  HC-CDR2 of antibody 2-G20-A
IYPYNDGT

SEQ ID NO: 308 - protein, 13 aa, Artificial
  HC-CDR3 of antibody 2-G20-A
ARGDYGDSAWFPY

SEQ ID NO: 309 - protein, 10 aa, Artificial
  LC-CDR1 of antibody 2-G20-A
KSVSTSGYSY

SEQ ID NO: 310 - protein, 3 aa, Artificial
  LC-CDR2 of antibody 2-G20-A
LAS

SEQ ID NO: 311 - protein, 9 aa, Artificial
  LC-CDR3 of antibody 2-G20-A
QLSRELPWT

SEQ ID NO: 312 - protein, 120 aa, Artificial
  Heavy chain variable domain of antibody 3-E2-A
EVQLQQSGPELVKPGASVKISCKASGYTFTDYYMNWVKQSHGKSLEWIGDINPNNGGTSYTQKFKGKATLTVDKSSSKAYMELRSLTSEDSAVYYCARRGDGHYGGFTYWGQGTLVTVSA

SEQ ID NO: 313 - DNA, 360 bp, Artificial
  Heavy chain variable domain of antibody 3-E2-A
gaggtccagctgcaacaatctggacctgaactggtgaagcctggggcttcagtgaagatatcctgtaaggcttctggatacacgttcactgactactacatgaactgggtgaagcagagccatggaaagagccttgagtggattggagatattaatcctaacaatggtggtactagctacacccagaagttcaagggcaaggccacattgactgtagacaagtcctccagcaaagcctacatggagctccgcagcctgacatctgaggactctgcagtctattactgtgcaagaagaggggatggtcactacgggggatttacttactggggccaagggactctggtcactgtctctgca

SEQ ID NO: 314 - protein, 111 aa, Artificial
  Light chain variable domain of antibody 3-E2-A
DIVLTQSPASLVVSLGQRATISCRASESVDNSGISFMNWFQQKPGQPPKLLIYAASNQGSGVPARFSGSGSGTDFSLNIHPMEEDDTAMYFCQQSKEVPWTFGGGTKLEIK

SEQ ID NO: 315 - DNA, 333 bp, Artificial
  Light chain variable domain of antibody 3-E2-A
gacattgtgctgacccaatctccagcttctttggttgtgtctctagggcagagggccaccatctcctgcagagccagcgaaagtgttgataattctggcattagttttatgaactggttccaacagaaaccaggacagccacccaaactcctcatctatgctgcatccaaccaaggatccggggtccctgccaggtttagtggcagtgggtctgggacagacttcagcctcaacatccatcctatggaggaggatgatactgcaatgtatttctgtcagcaaagtaaggaggttccgtggacgttcggtggaggcaccaagctggaaatcaaa

SEQ ID NO: 316 - protein, 8 aa, Artificial
  HC-CDR1 of antibody 3-E2-A
GYTFTDYY

SEQ ID NO: 317 - protein, 8 aa, Artificial
  HC-CDR2 of antibody 3-E2-A
INPNNGGT

SEQ ID NO: 318 - protein, 13 aa, Artificial
  HC-CDR3 of antibody 3-E2-A
ARRGDGHYGGFTY

SEQ ID NO: 319 - protein, 10 aa, Artificial
  LC-CDR1 of antibody 3-E2-A
ESVDNSGISF

SEQ ID NO: 320 - protein, 3 aa, Artificial
  LC-CDR2 of antibody 3-E2-A
AAS

SEQ ID NO: 321 - protein, 9 aa, Artificial
  LC-CDR3 of antibody 3-E2-A
QQSKEVPWT

SEQ ID NO: 322 - protein, 114 aa, Artificial
  Heavy chain variable domain of antibody 4-K16-A
QIQLVQSGPELKKPGETVKISCKASGYTFTTYGMSWVKQAPGKGLKWMGWMSTYSGVPTYADDFKGRFAFSLETSASTAYLQINNLKNEDTATYFCASGDVGYWGQGTTLTVSS

SEQ ID NO: 323 - DNA, 342 bp, Artificial
  Heavy chain variable domain of antibody 4-K16-A
cagatccagttggtacagtctggacctgagctgaagaagcctggagagacagtcaagatctcctgcaaggcttctgggtataccttcacaacctatggaatgagctgggtgaaacaggctccaggaaagggtttaaagtggatgggctggatgagcacctactctggagtgccaacatatgctgatgacttcaagggacggtttgccttctctttggaaacctctgccagcactgcctatttgcagatcaacaacctcaaaaatgaggacacggctacatatttctgtgcaagcggggacgtcggctactggggccaaggcaccactctcacagtctcctca

SEQ ID NO: 324 - protein, 107 aa, Artificial
  Light chain variable domain of antibody 4-K16-A
DIVMTQSQKFMSTSVGDRVSVTCKASQNVGTNVAWYQQKPGQSPKALIYSASYRYSGVPDRFTGSGSGTDFTLTISTVQSEDLAEYFCQQYNIYPSTFGGGTKLEIK

SEQ ID NO: 325 - DNA, 321 bp, Artificial
  Light chain variable domain of antibody 4-K16-A
gacattgtgatgacccagtctcaaaaattcatgtccacatcagtaggagacagggtcagcgtcacctgcaaggccagtcagaatgtgggtactaatgtagcctggtatcaacagaaaccagggcaatctcctaaagcactgatttactcggcatcctaccggtacagtggagtccctgatcgcttcacaggcagtggatctgggacagatttcactctcaccatcagcactgtgcagtctgaagacttggcagagtatttctgtcagcaatataacatctatccgtccacgttcggaggggggaccaagctggaaataaaa

SEQ ID NO: 326 - protein, 8 aa, Artificial
  HC-CDR1 of antibody 4-K16-A
GYTFTTYG

SEQ ID NO: 327 - protein, 8 aa, Artificial
  HC-CDR2 of antibody 4-K16-A
MSTYSGVP

SEQ ID NO: 328 - protein, 7 aa, Artificial
  HC-CDR3 of antibody 4-K16-A
ASGDVGY

SEQ ID NO: 329 - protein, 6 aa, Artificial
  LC-CDR1 of antibody 4-K16-A
QNVGTN

SEQ ID NO: 330 - protein, 3 aa, Artificial
  LC-CDR2 of antibody 4-K16-A
SAS

SEQ ID NO: 331 - protein, 9 aa, Artificial
  LC-CDR3 of antibody 4-K16-A
QQYNIYPST

SEQ ID NO: 332 - protein, 124 aa, Artificial
  Heavy chain variable domain of antibody 6-C19-A
EVQLQQSGPELVKPGASVKMSCKASGYTFTDYNIHWVKQSHGKSLEWIGYINPNNGGTDYNQKFQGKATLTVDKSSSTAYMELRSLTSEDSAVYYCARGGFYGNYGSYYGMDFWGQGTSVTVSS

SEQ ID NO: 333 - DNA, 372 bp, Artificial
  Heavy chain variable domain of antibody 6-C19-A
gaggtccagctgcaacagtctggacctgagctggtgaagcctggggcttcagtgaagatgtcctgcaaggcttctggatacacattcactgactacaacatacactgggtgaagcagagccatggaaagagccttgagtggattggatatattaaccctaacaatggtggtactgactacaaccagaagttccagggcaaggccacattgactgtagacaagtcctccagcacagcctacatggagctccgcagcctgacatcggaagattctgcagtctattactgtgcaagaggggggttttatggcaattacgggtcttactatggtatggacttctggggtcaaggaacctcagtcaccgtctcctca

SEQ ID NO: 334 - protein, 107 aa, Artificial
  Light chain variable domain of antibody 6-C19-A
DIQMTQSPASLSASVGETVTITCRTSENIYSHLAWYQQTQGKSPHLLVYNAKTLAEGVPSRFSGSGSGTQFSLKINSLQPEDFGSYFCQHHYGIPPEFGGGTKLEIK

SEQ ID NO: 335 - DNA, 321 bp, Artificial
  Light chain variable domain of antibody 6-C19-A
gacatccagatgactcagtctccagcctccctatctgcatctgtgggagaaactgtcaccatcacatgtcgaacaagtgagaatatttatagtcatttagcatggtatcagcagacacagggaaaatctcctcacctcctggtctataatgcaaaaaccttagcagaaggtgtgccatcaaggttcagtggcagtggatcaggcacacagttttctctgaagatcaacagcctgcagcctgaagattttgggagttatttctgtcaacatcattatggtattcctccggagttcggtggaggcaccaagctggaaatcaaa

SEQ ID NO: 336 - protein, 8 aa, Artificial
  HC-CDR1 of antibody 6-C19-A
GYTFTDYN

SEQ ID NO: 337 - protein, 8 aa, Artificial
  HC-CDR2 of antibody 6-C19-A
INPNNGGT

SEQ ID NO: 338 - protein, 17 aa, Artificial
  HC-CDR3 of antibody 6-C19-A
ARGGFYGNYGSYYGMDF

SEQ ID NO: 339 - protein, 6 aa, Artificial
  LC-CDR1 of antibody 6-C19-A
ENIYSH

SEQ ID NO: 340 - protein, 3 aa, Artificial
  LC-CDR2 of antibody 6-C19-A
NAK

SEQ ID NO: 341 - protein, 9 aa, Artificial
  LC-CDR3 of antibody 6-C19-A
QHHYGIPPE

SEQ ID NO: 342 - protein, 117 aa, Artificial
  Heavy chain variable domain of antibody 6-L8-A
EVQLQQSVAELVRPGASVKLSCTPSGFNIKNTYMHWMKQRPEQGLEWIGRINPANDDTKYAPKFQDKATITADTSSNTAYLQLSSLTSEDTAIYYCVYDVRWYFDVWGTGTTVTVSS

SEQ ID NO: 343 - DNA, 351 bp, Artificial
  Heavy chain variable domain of antibody 6-L8-A
gaggttcagctgcagcagtctgtggcagagcttgtgaggccaggggcctcagtcaagttgtcctgcacaccttctggcttcaacattaaaaacacctatatgcactggatgaagcagaggcctgaacagggcctggagtggattggaaggattaatcctgcgaatgatgatactaaatatgccccgaagttccaggacaaggccactataactgcagacacatcctccaacacagcctacctgcagctcagcagcctgacatctgaggacactgccatctattactgtgtctacgacgtacggtggtatttcgatgtctggggcacagggaccacggtcaccgtctcctca

SEQ ID NO: 344 - protein, 112 aa, Artificial
  Light chain variable domain of antibody 6-L8-A
DVVMTQTPLSLPVSLGDQASISCRSSQGLVHSNGNTYLHWYLQKPGQSPKLLIYKVSNRFSGVPDRFSGSGSGTDFTLKISRVEAEDLGVYFCSQSTHVPPTFGAGTKLELK

SEQ ID NO: 345 - DNA, 336 bp, Artificial
  Light chain variable domain of antibody 6-L8-A
gatgttgtgatgacccaaactccactctccctgcctgtcagtcttggagatcaagcctccatctcttgcagatctagtcagggccttgtacacagtaatgggaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttcctcctacgttcggtgctgggaccaagctggagctgaaa

SEQ ID NO: 346 - protein, 8 aa, Artificial
  HC-CDR1 of antibody 6-L8-A
GFNIKNTY

SEQ ID NO: 347 - protein, 8 aa, Artificial
  HC-CDR2 of antibody 6-L8-A
INPANDDT

SEQ ID NO: 348 - protein, 10 aa, Artificial
  HC-CDR3 of antibody 6-L8-A
VYDVRWYFDV

SEQ ID NO: 349 - protein, 11 aa, Artificial
  LC-CDR1 of antibody 6-L8-A
QGLVHSNGNTY

SEQ ID NO: 350 - protein, 3 aa, Artificial
  LC-CDR2 of antibody 6-L8-A
KVS

SEQ ID NO: 351 - protein, 9 aa, Artificial
  LC-CDR3 of antibody 6-L8-A
SQSTHVPPT

SEQ ID NO: 352 - protein, 115 aa, Artificial
  Heavy chain variable domain of antibody 7-D7-A
QVQLKESGPGLVAPSQSLSITCTVSGFSLTSYAISWVRQPPGKGLEWLGVIWPGGGTNYNSALKSRLSISKDNSKSQVFLKMNSLQTDDTAKYYCARTGYYFDYWGQGTTLTVSS

SEQ ID NO: 353 - DNA, 345 bp, Artificial
  Heavy chain variable domain of antibody 7-D7-A
caggtgcagctgaaggagtcaggacctggcctggtggcgccctcacagagcctgtccatcacatgcactgtctctgggttctcattaaccagctatgctataagctgggttcgccagccaccaggaaagggtctggagtggcttggagtaatatggcctggtggaggcacaaattataattcagctctcaaatccagactgagcatcagcaaagacaactccaagagtcaagttttcttaaaaatgaacagtctgcaaactgatgacacagccaaatactactgtgccagaactgggtactactttgactactggggccaaggcaccactctcacagtctcctca

SEQ ID NO: 354 - protein, 108 aa, Artificial
  Light chain variable domain of antibody 7-D7-A
QLILTQSPAIMSASPGEKVTMTCSASLSVNYMYWFQQKPGTSPKRWIHDTSKLASGVPARFSGSGSGTSYSLTISSMEAEDAATYYCQQWSSNPPMYTFGGGTKLEIK

SEQ ID NO: 355 - DNA, 324 bp, Artificial
  Light chain variable domain of antibody 7-D7-A
caacttattctcacccagtctccagcaatcatgtctgcatctccaggggagaaggtcaccatgacctgcagtgccagcttaagtgtaaattacatgtactggttccagcagaagccaggcacctcccccaaaagatggattcatgacacatccaaactggcttctggagtccctgctcgcttcagtggcagtgggtctgggacctcttactctctcacaatcagcagcatggaggctgaagatgctgccacttattactgccagcagtggagtagtaacccacccatgtacacgttcggaggggggaccaagctggaaataaaa

SEQ ID NO: 356 - protein, 8 aa, Artificial
  HC-CDR1 of antibody 7-D7-A
GFSLTSYA

SEQ ID NO: 357 - protein, 7 aa, Artificial
  HC-CDR2 of antibody 7-D7-A
IWPGGGT

SEQ ID NO: 358 - protein, 9 aa, Artificial
  HC-CDR3 of antibody 7-D7-A
ARTGYYFDY

SEQ ID NO: 359 - protein, 5 aa, Artificial
  LC-CDR1 of antibody 7-D7-A
LSVNY

SEQ ID NO: 360 - protein, 3 aa, Artificial
  LC-CDR2 of antibody 7-D7-A
DTS

SEQ ID NO: 361 - protein, 11 aa, Artificial
  LC-CDR3 of antibody 7-D7-A
QQWSSNPPMYT

SEQ ID NO: 362 - protein, 120 aa, Artificial
  Heavy chain variable domain of antibody 7-N20-A
QVQLVETGGGLVRPGNSLKLSCVTSGFTFSNYRMHWLRQPPGKRLEWIGIITDKSDNYGANYAESVKGRFAISRDDSKSSVYLEMKRLREEDTATYFCSRDGYDWYFDLWGTGTTVTVSS

SEQ ID NO: 363 - DNA, 360 bp, Artificial
  Heavy chain variable domain of antibody 7-N20-A
caggtgcagcttgtagagaccgggggaggcttggtgaggcctggaaattctctgaaactctcctgtgttacgtcgggattcactttcagtaactaccggatgcactggcttcgccagcctccagggaagaggctggagtggattggtataattacagacaaatctgataattatggagcaaattatgcagagtctgtgaaaggcagattcgccatttcaagagatgattcaaaaagtagtgtctatctagagatgaagagattaagagaggaagacactgccacttatttttgtagtagagatggttacgactggtacttcgatctctggggcacagggaccacggtcaccgtctcctca

SEQ ID NO: 364 - protein, 106 aa, Artificial
  Light chain variable domain of antibody 7-N20-A
DIVMTQSHKFMSTSVGDRVRITCKASQDVGIVVAWYQQKPGQSPKLLIYWASTRHTGVPDRFTGSGSGTDFTLTISNVQSEDLADYFCQQYSSYPTFGGGTKLEIK

SEQ ID NO: 365 - DNA, 318 bp, Artificial
  Light chain variable domain of antibody 7-N20-A
gacattgtgatgacccagtctcacaaattcatgtccacatcagtaggagacagggtcagaatcacctgtaaggccagtcaggatgtgggaattgttgtagcctggtatcaacagaaaccagggcaatctcctaaactactgatttactgggcatccacccggcacactggagtccctgatcgcttcacaggcagtggatctgggacagatttcactctcaccattagcaatgtgcagtctgaagacttggcagattatttctgtcagcaatatagcagctatcccacgttcggaggggggaccaagctggaaataaaa

SEQ ID NO: 366 - protein, 8 aa, Artificial
  HC-CDR1 of antibody 7-N20-A
GFTFSNYR

SEQ ID NO: 367 - protein, 10 aa, Artificial
  HC-CDR2 of antibody 7-N20-A
ITDKSDNYGA

SEQ ID NO: 368 - protein, 11 aa, Artificial
  HC-CDR3 of antibody 7-N20-A
SRDGYDWYFDL

SEQ ID NO: 369 - protein, 6 aa, Artificial
  LC-CDR1 of antibody 7-N20-A
QDVGIV

SEQ ID NO: 370 - protein, 3 aa, Artificial
  LC-CDR2 of antibody 7-N20-A
WAS

SEQ ID NO: 371 - protein, 8 aa, Artificial
  LC-CDR3 of antibody 7-N20-A
QQYSSYPT

SEQ ID NO: 372 - protein, 115 aa, Artificial
  Heavy chain variable domain of antibody 8-A17-A
QVQLKESGPGLVAPSQSLSITCTVSGFSLTSYAISWVRQPPGKGLEWLGVVWPGGGTNYNSALKSRLSINKDNSKSQVFLKMNSLQTDDTGRYYCARTGYHFDYWGQGTTLTVSS

SEQ ID NO: 373 - DNA, 345 bp, Artificial
  Heavy chain variable domain of antibody 8-A17-A
caggtgcagctgaaggagtcaggacctggcctggtggcgccctcacagagcctgtccatcacatgcactgtctctgggttctcattaaccagctatgctataagctgggttcgccagccaccaggaaagggtctggagtggcttggagtagtatggcctggtggaggcacaaattataattcagctctcaaatccagactgagcatcaacaaagacaactccaagagtcaagttttcttaaagatgaacagtctgcaaactgatgacacaggcaggtactactgtgccagaactgggtaccattttgactactggggccaaggcaccactctcacagtctcctca

SEQ ID NO: 374 - protein, 108 aa, Artificial
  Light chain variable domain of antibody 8-A17-A
QIVLTQSPAIMSASPGEKVTMTCSVSSSVSYMHWYQQKSGTSPKRWIYDTSKLASGVPARFSGSGSGTSYSLTISSMEAEDAATYYCQQWSSNPPMYTFGGGTKLEIK

SEQ ID NO: 375 - DNA, 324 bp, Artificial
  Light chain variable domain of antibody 8-A17-A
caaattgttctcacccagtctccagcaatcatgtctgcatctccaggggagaaggtcaccatgacctgcagtgtcagctcaagtgtaagttacatgcactggtaccagcagaagtcaggcacctcccccaaaagatggatttatgacacatccaaactggcttctggagtccctgctcgtttcagtggcagtgggtctgggacctcttactctctcacaatcagcagcatggaggctgaagatgctgccacttattactgccagcagtggagtagtaacccacccatgtacacgttcggaggggggaccaagctggaaataaaa

SEQ ID NO: 376 - protein, 8 aa, Artificial
  HC-CDR1 of antibody 8-A17-A
GFSLTSYA

SEQ ID NO: 377 - protein, 7 aa, Artificial
  HC-CDR2 of antibody 8-A17-A
VWPGGGT

SEQ ID NO: 378 - protein, 9 aa, Artificial
  HC-CDR3 of antibody 8-A17-A
ARTGYHFDY

SEQ ID NO: 379 - protein, 5 aa, Artificial
  LC-CDR1 of antibody 8-A17-A
SSVSY

SEQ ID NO: 380 - protein, 3 aa, Artificial
  LC-CDR2 of antibody 8-A17-A
DTS

SEQ ID NO: 381 - protein, 11 aa, Artificial
  LC-CDR3 of antibody 8-A17-A
QQWSSNPPMYT

SEQ ID NO: 382 - protein, 124 aa, Artificial
  Heavy chain variable domain of antibody 8-H3-A
EVQLVESGGGLVQPKGSLKLSCAASGFTFNTYAMHWVRQAPGKGLEWVARIRSKSSNYATYYADSVKDRFTISRDDSQSMLYLQMNNLKTEDTAMYYCVRDHDYYGSSYWFTYWGQGTLVTVSA

SEQ ID NO: 383 - DNA, 372 bp, Artificial
  Heavy chain variable domain of antibody 8-H3-A
gaggtgcagcttgttgagtctggtggaggattggtgcagcctaaaggatcattgaaactctcatgtgccgcctctggtttcaccttcaatacctatgccatgcactgggtccgccaggctccaggaaagggtttggaatgggttgctcgcataagaagtaaaagtagtaattatgcaacatattatgccgattcagtgaaagacagattcaccatctccagagatgattcacaaagcatgctctatctgcaaatgaacaacctgaaaactgaggacacggccatgtattactgtgtgagagatcacgattactacggtagtagctactggtttacttactggggccaagggactctggtcactgtctctgca

SEQ ID NO: 384 - protein, 112 aa, Artificial
  Light chain variable domain of antibody 8-H3-A
DVVMTQTPLSLPVSLGDQASISCRSSQSLVHDNGNTYLHWFLQKPGQSPKLLIYKVSNRFSGVPDRFSGSGSGTDFTLKISRVETEDLGVYFCSQSTHVPWTFGGGTKLEIK

SEQ ID NO: 385 - DNA, 336 bp, Artificial
  Light chain variable domain of antibody 8-H3-A
gatgttgtgatgacccaaactccactctccctgcctgtcagtcttggagatcaagcctccatctcttgcagatctagtcagagccttgtacacgataatggaaacacctatttacattggttcctgcagaagccaggccagtctccaaagctcctgatctacaaagtttccaaccgattttctggggtcccagacaggttcagtggcagtggatcagggacagatttcacactcaagatcagcagagtggagactgaggatctgggagtttatttctgctctcaaagtacacatgttccgtggacgttcggtggaggcaccaagctggaaatcaaa

SEQ ID NO: 386 - protein, 8 aa, Artificial
  HC-CDR1 of antibody 8-H3-A
GFTFNTYA

SEQ ID NO: 387 - protein, 10 aa, Artificial
  HC-CDR2 of antibody 8-H3-A
IRSKSSNYAT

SEQ ID NO: 388 - protein, 15 aa, Artificial
  HC-CDR3 of antibody 8-H3-A
VRDHDYYGSSYWFTY

SEQ ID NO: 389 - protein, 11 aa, Artificial
  LC-CDR1 of antibody 8-H3-A
QSLVHDNGNTY

SEQ ID NO: 390 - protein, 3 aa, Artificial
  LC-CDR2 of antibody 8-H3-A
KVS

SEQ ID NO: 391 - protein, 9 aa, Artificial
  LC-CDR3 of antibody 8-H3-A
SQSTHVPWT

SEQ ID NO: 392 - protein, 124 aa, Artificial
  Heavy chain variable domain of antibody 8-L17-A
EVQLVESGGGLVQPKGSLKLSCAASGFTFNTYAMHWVRQAPGKGLEWVARIRSKSSNYARYYADSVKDRFTISRDDSQSMLYLQMNNLKTEDTAMYYCVRDHDYYGSSYWFAYWGQGTLVTVSA

SEQ ID NO: 393 - DNA, 372 bp, Artificial
  Heavy chain variable domain of antibody 8-L17-A
gaggtgcagcttgttgagtctggtggaggattggtgcagcctaaaggatcattgaaactctcatgtgccgcctctggtttcaccttcaatacctatgccatgcactgggtccgccaggctccaggaaagggtttggaatgggttgctcgcataagaagtaaaagtagtaattatgcaagatattatgccgattcagtgaaagacagattcaccatctccagagatgattcacaaagcatgctctatctgcaaatgaacaacctgaaaactgaggacacagccatgtattactgtgtgagagatcacgattactacggtagtagttactggtttgcttactggggccaagggactctggtcactgtctctgca

SEQ ID NO: 394 - protein, 112 aa, Artificial
  Light chain variable domain of antibody 8-L17-A
DVVMTQTPLSLPVSLGDQASISCRSSQSLVHNNGNTYLHWYLQKPGQSPKLLIYKVSNRFSGVPDRFSGSGSGTDFTLKISRVEAEDLGVYFCSQSTHVPWTFGGGTKLEIK

SEQ ID NO: 395 - DNA, 336 bp, Artificial
  Light chain variable domain of antibody 8-L17-A
gatgttgtgatgacccaaactccactctccctgcctgtcagtcttggagatcaagcctccatctcttgcagatctagtcagagccttgtacacaataatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtggacgttcggtggaggcaccaagctggaaatcaaa

SEQ ID NO: 396 - protein, 8 aa, Artificial
  HC-CDR1 of antibody 8-L17-A
GFTFNTYA

SEQ ID NO: 397 - protein, 10 aa, Artificial
  HC-CDR2 of antibody 8-L17-A
IRSKSSNYAR

SEQ ID NO: 398 - protein, 15 aa, Artificial
  HC-CDR3 of antibody 8-L17-A
VRDHDYYGSSYWFAY

SEQ ID NO: 399 - protein, 11 aa, Artificial
  LC-CDR1 of antibody 8-L17-A
QSLVHNNGNTY

SEQ ID NO: 400 - protein, 3 aa, Artificial
  LC-CDR2 of antibody 8-L17-A
KVS

SEQ ID NO: 401 - protein, 9 aa, Artificial
  LC-CDR3 of antibody 8-L17-A
SQSTHVPWT

SEQ ID NO: 402 - protein, 118 aa, Artificial
  Heavy chain variable domain of antibody 9-F6-A
EVQLQQSGPELVKPGASVKMSCKASGYTFTSYIMHWVKQKPGQGLEWIGYILPYNDGTKYNEKFKGKATLTSDKSSSTAYMELRSLTSEDSAVYYCARWGYSNFLDSWGQGTTLTVSS

SEQ ID NO: 403 - DNA, 354 bp, Artificial
  Heavy chain variable domain of antibody 9-F6-A
gaggtccagctgcagcagtctggacctgagctggtaaagcctggggcttcagtgaagatgtcctgcaaggcttctggatacacattcactagctatattatgcactgggtgaagcagaagcctggacagggccttgagtggattggatatattcttccttacaatgatggtactaagtacaatgagaagttcaaaggcaaggccacactgacttcagacaaatcctccagcacagcctatatggagctcagaagcctgacctctgaggactctgcggtctattactgtgcaagatggggctatagtaactttcttgactcctggggccaaggcaccactctcacagtctcctca

SEQ ID NO: 404 - protein, 107 aa, Artificial
  Light chain variable domain of antibody 9-F6-A
DIQMTQTTSSLSASLGDRVTISCRASQDINNYLNWYQQKPDGTVKLLIYYTSRLHSGVPSRFSGSGSGTDYSLTIRNLEQEDIATYFCQQGNTLPPTFGGGTKLEIK

SEQ ID NO: 405 - DNA, 321 bp, Artificial
  Light chain variable domain of antibody 9-F6-A
gatatccagatgacacagactacatcctccctgtctgcctctctgggagacagagtcaccatcagttgcagggcaagtcaggacattaacaattatttaaactggtatcagcagaaaccagatggaactgttaaactcctgatctactacacatcaagattacactcaggagtcccatcaaggttcagtggcagtgggtctggaacagattattctctcaccattaggaacctggagcaagaagacattgccacttacttttgccaacagggtaatacgcttcctccgacgttcggtggaggcaccaagctggaaatcaaa

SEQ ID NO: 406 - protein, 8 aa, Artificial
  HC-CDR1 of antibody 9-F6-A
GYTFTSYI

SEQ ID NO: 407 - protein, 8 aa, Artificial
  HC-CDR2 of antibody 9-F6-A
ILPYNDGT

SEQ ID NO: 408 - protein, 11 aa, Artificial
  HC-CDR3 of antibody 9-F6-A
ARWGYSNFLDS

SEQ ID NO: 409 - protein, 6 aa, Artificial
  LC-CDR1 of antibody 9-F6-A
QDINNY

SEQ ID NO: 410 - protein, 3 aa, Artificial
  LC-CDR2 of antibody 9-F6-A
YTS

SEQ ID NO: 411 - protein, 9 aa, Artificial
  LC-CDR3 of antibody 9-F6-A
QQGNTLPPT

SEQ ID NO: 412 - protein, 119 aa, Artificial
  Heavy chain variable domain of antibody 10-I12-A
RVQLQQSGPELVRPGTSVKISCKASGYTFTDSYINWVTQRPGQGLDWIGWIFPGNDNTYYNEKFEGKATLTLDKSSSTAYMLLSSLTSEDSAVYFCARSGGLRLGFAYWGQGTLVTVSA

SEQ ID NO: 413 - DNA, 357 bp, Artificial
  Heavy chain variable domain of antibody 10-I12-A
cgggtccagctacagcagtctggacctgagctggtgaggcctgggacttcagtgaagatatcctgcaaggcttctggctacaccttcactgactcctatataaactgggtgacgcagaggcctggacagggacttgattggattggatggatttttcctggaaatgataatacttactacaatgagaagttcgagggcaaggccacacttactcttgacaaatcttccagtacagcctacatgttgctcagcagcctgacctctgaggactctgcggtctatttctgtgcaagatcagggggattacgactggggtttgcttactggggccaagggactctggtcactgtctctgca

SEQ ID NO: 414 - protein, 111 aa, Artificial
  Light chain variable domain of antibody 10-I12-A
NIVLTQSPASLAVSRGQRATISCRASESVDSYGHSFIHWYQQKPGQPPKILIYLASKLESGVPARFSGSGSRTDFTLTIDPVEADDAATYYCQQNNEDPWTFGGGTKLEIK

SEQ ID NO: 415 - DNA, 333 bp, Artificial
  Light chain variable domain of antibody 10-I12-A
aacattgtgctgacccaatctccagcttctttggctgtgtctcgagggcagagggccaccatatcctgcagagccagtgaaagtgttgatagttatggccatagttttattcactggtaccagcagaaaccaggacagccacccaaaatcctcatctatcttgcatccaagttagaatctggggtccctgccaggttcagtggcagtgggtctaggacagacttcaccctcaccattgatcctgtggaggctgatgatgctgcaacctattactgtcagcaaaataatgaggatccgtggacgttcggtggaggcaccaagctggaaatcaaa

SEQ ID NO: 416 - protein, 8 aa, Artificial
  HC-CDR1 of antibody 10-I12-A
GYTFTDSY

SEQ ID NO: 417 - protein, 8 aa, Artificial
  HC-CDR2 of antibody 10-I12-A
IFPGNDNT

SEQ ID NO: 418 - protein, 12 aa, Artificial
  HC-CDR3 of antibody 10-I12-A
ARSGGLRLGFAY

SEQ ID NO: 419 - protein, 10 aa, Artificial
  LC-CDR1 of antibody 10-I12-A
ESVDSYGHSF

SEQ ID NO: 420 - protein, 3 aa, Artificial
  LC-CDR2 of antibody 10-I12-A
LAS

SEQ ID NO: 421 - protein, 9 aa, Artificial
  LC-CDR3 of antibody 10-I12-A
QQNNEDPWT

SEQ ID NO: 422 - protein, 214 aa, Artificial
  LC of antibody B13
DIQMTQTTSSLSASLGDRVTISCRASQDISNYLNWYQQKPDGTVKLLIYYTSRLQSGVPSRFSGSGTGTDYSLTISNLEQEDIATYFCQQGNTLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 423 - protein, 450 aa, Artificial
  HC of antibody B13
QVQLQQSGAELVKPGASVKISCKASGYAFSNYWMNWVKQRPGKGLEWIGQIYPGDGDTNYNGNFKGKATLTADKSSSTAYIQLSSLTSEDSAVYFCGRGFIATVVEVFDHWGLGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 424 - protein, 218 aa, Artificial
  LC of antibody O24
DIVLTQSPASLAVFLGQRATISCRASKSVSTSGYSYMHWYQQRPGQPPKLLIYLASNLESGVPARFSGSGSGTDFTLNIHPVEEEDAATYYCQHSRELPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 425 - protein, 447 aa, Artificial
  HC of antibody O24
QAYLQQSGAELVRPGASVKMSCKASGYTFTSYSMHWVKQTPRQGLEWIGAIYPGNDDTSYNQKFKGKATLTVDRSSSTAYMQLSSLTSEDSAVYFCTRDYSNYPFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG